(19) United States
(12) Patent Application Publication
(10) Pub. No.: US 2004/0067216 A1
(43) Pub. Date: Apr. 8, 2004

(54) Hiv protease inhibitors supported on cation exchange resins for oral administration

(76) Inventor: Shyam B. Karki, Lansdale, PA (US)

Correspondence Address: MERCK AND CO INC, P O BOX 2000, RAHWAY, NJ, 070650907

(21) Appl. No.: 10/468,429

(22) PCT Filed: Feb. 22, 2002
(86) PCT No.: PCT/US02/05544

(51) Int. Cl. A61K 31/785; A61K 31/495

(52) U.S. Cl. 424/78.14; 514/255.01; 514/252.13

(57) ABSTRACT

γ-Hydroxy-2-(fluoroalkylaminocarbonyl)-1-piperazinepentanamide compounds useful for inhibiting HIV protease and HIV replication and useful in the prevention or treatment of infection by HIV and the treatment of AIDS are complexed with cation exchange resins for oral administration.

### FIELD OF THE INVENTION

The present invention is directed to pharmaceutical compositions suitable for oral administration which comprise a cation exchange resin complexed with a γ-hydroxy-2-(fluoroalkylaminocarbonyl)-1-piperazinepentanamide compound or a pharmaceutically acceptable salt thereof. The compositions of the present invention are useful for inhibiting HIV protease, for preventing or treating infection by HIV, and for treating or delaying the onset of AIDS.

### BACKGROUND OF THE INVENTION

A retrovirus designated human immunodeficiency virus (HIV) is the etiological agent of the complex disease that includes progressive destruction of the immune system (acquired immune deficiency syndrome; AIDS) and degeneration of the central and peripheral nervous system. This virus was previously known as LAV, HTLV-III, or ARV. A common feature of retrovirus replication is the extensive post-translational processing of precursor polyproteins by a virally encoded protease to generate mature viral proteins required for virus assembly and function. Inhibition of this processing prevents the production of normally infectious virus. For example, Kohl et al., Proc. Nat'l Acad. Sci. 1988, 85: 4686, demonstrated that genetic inactivation of the HIV encoded protease resulted in the production of immature, non-infectious virus particles. These results indicated that inhibition of the HIV protease represents a viable method for the treatment of AIDS and the prevention or treatment of infection by HIV.

Nucleotide sequencing of HIV shows the presence of a pol gene in one open reading frame [Ratner et al., Nature 1985, 313: 277]. Amino acid sequence homology provides evidence that the pol sequence encodes reverse transcriptase, an endonuclease and an HIV protease [Toh et al., EMBO J. 1985, 4: 1267; Power et al., Science 1986, 231: 1567; Pearl et al., Nature 1987, 329: 351].

Several HIV protease inhibitors are presently in clinical use for the treatment of AIDS and HIV infection, including indinavir (see U.S. Pat. No. 5,413,999), nelfinavir (U.S. Pat. No. 5,484,926), saquinavir (U.S. Pat. No. 5,196,438), and ritonavir (U.S. 5,484,801). Each of these protease inhibitors is a peptidomimetic, competitive inhibitor of the viral protease which prevents cleavage of the HIV gag-pol polyprotein precursor. Indinavir, for example, has been found to be highly effective in reducing HIV viral loads and increasing CD4 cell counts in HIV-infected patients, when used in combination with nucleoside reverse transcriptase inhibitors. See, for example, Hammer et al., New England J. Med. 1997, 337: 725-733 and Gulick et al., New England J. Med. 1997, 337: 734-739.

A novel group of γ-hydroxy-2-(fluoroalkylaminocarbonyl)-1-piperazinepentanamide compounds have recently been prepared which are potent inhibitors of HIV protease including mutant forms thereof that are resistant to known protease inhibitors. These compounds are disclosed in WO 01/38332. Exemplifying this group of compounds is (αR,γS,2S)-4-[[5-(5-chloro-2-pyridinyl)-2-furanyl]methyl]-N-((3S,4S)-3,4-dihydro-3-hydroxy-2H-1-benzopyran-4-yl)-γ-hydroxy-α-(phenylmethyl)-2-[[(2,2,2-trifluoroethyl)amino]-carbonyl]-1-piperazinepentanamide, hereinafter alternatively referred as Compound A. The compounds of this class are relatively weak bases for which crystalline salts can be difficult to isolate. Crystalline salts are typically desirable for use in pharmaceutical formulations, because they often have greater chemical and physical stability than the corresponding free base material or amorphous salts thereof, and the use of a relatively unstable form of an active ingredient (e.g., a form whose properties can be difficult to quantify and which can change with time) can adversely and unpredictably affect the safety and efficacy of a dosage regimen. The preparation of stable crystalline salts of Compound A, for example, has proved elusive. The typical acid salts of Compound A (e.g., the chloride, bromide, and sulfate salts, and many others) have not been prepared in crystalline form. Furthermore, the non-crystalline salts which have been prepared have been found to disproportionate to the free base when added to water. The free base administered in solid form (e.g., powder) has poor oral bioavailability in animals such as rats and dogs. Suspensions of Compound A free base in Methocel® (methylcellulose; Dow Chemical Co., Midland, Mich.) also have poor absorption in animals. Acidified propylene glycol solutions of Compound A have improved oral bioavailability, but a relatively low pH is required to keep the compound in solution. Because the low pH can result in chemical stability problems (e.g., lactonization of Compound A), glycol solutions are not a desirable formulation for oral administration. The use of semi-solid formulations of the free base in soft gel capsules or hard gelatin capsules also require an acidic environment which can result in stability problems. In addition relatively expensive, specialized filling instrumentation is required to make the semi-solids. In summary, pharmaceutical compositions incorporating these protease inhibitors which are relatively stable solids suitable for oral administration and which exhibit good bioavailability would be an attractive alternative to compositions employing the free base or amorphous salt forms of the compounds and compositions employing liquid solutions of the compounds.

### SUMMARY OF THE INVENTION

The present invention provides pharmaceutical compositions suitable for oral administration which comprise complexes of a γ-hydroxy-2-(fluoroalkylaminocarbonyl)-1-piperazinepentanamide compound with a cation exchange resin. These complexes are typically free-flowing solids that can be formulated via conventional techniques (e.g., encapsulation) and can have better thermal stability and/or improved bioavailibility relative to the solid free base and solutions thereof. More particularly, the present invention includes a pharmaceutical composition which comprises a cation exchange resin complexed with a compound of Formula (I) or a pharmaceutically acceptable salt thereof:

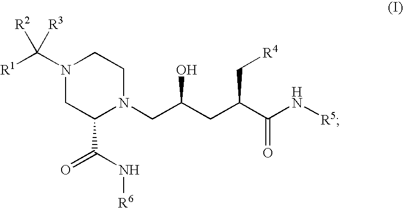

wherein

R¹ is C₁-C₆ alkyl, C₂-C₆ alkenyl, C₂-C₆ alkynyl, C₃-C₆ cycloalkyl, aryl, substituted aryl, heteroaryl, or substituted heteroaryl; wherein

(i) each of the substituents on substituted aryl is independently

(a) halogen,

(b) cyano,

(c) hydroxy,

(d) C₁-C₆ alkyl,

(e) C₂-C₆ alkenyl,

(f) C₂-C₆ alkynyl,

(g) fluorinated C₁-C₆ alkyl,

(h) C₁-C₆ alkoxy,

(i) fluorinated C₁-C₆ alkoxy,

(j) S-(C₁-C₆ alkyl),

(k) heterocycle, or

(l) heterocycle substituted with one or more substituents independently selected from halogen, cyano, hydroxy, C₁-C₆ alkyl, C₂-C₆ alkenyl, C₂-C₆ alkynyl, fluorinated C₁-C₆ alkyl, C₁-C₆ alkoxy, fluorinated C₁-C₆ alkoxy, S-(C₁-C₆ alkyl), and NR^{a}R^{b};

(ii) each of the substituents on substituted heteroaryl is independently

(a) halogen,

(b) cyano,

(c) hydroxy,

(d) NR^{a}R^{b},

(e) C₁-C₆ alkyl,

(f) C₂-C₆ alkenyl,

(g) C₂-C₆ alkynyl,

(h) fluorinated C₁-C₆ alkyl,

(i) C₁-C₆ alkoxy,

(j) fluorinated C₁-C₆ alkoxy,

(k) S-(C₁-C₆ alkyl),

(l) phenyl,

(m) phenyl substituted with one or more substituents independently selected from halogen, cyano, hydroxy, C₁-C₆ alkyl, C₂-C₆ alkenyl, C₂-C₆ alkynyl, fluorinated C₁-C₆ alkyl, C₁-C₆ alkoxy, fluorinated C₁-C₆ alkoxy, and S-(C₁-C₆ alkyl),

(l) heterocycle, or

(m) heterocycle substituted with one or more substituents independently selected from halogen, cyano, hydroxy, C₁-C₆ alkyl, C₂-C₆ alkenyl, C₂-C₆ alkynyl, fluorinated C₁-C₆ alkyl, C₁-C₆ alkoxy, fluorinated C₁-C₆ alkoxy, S-(C₁-C₆ alkyl), NR^{a}R^{b}, and a 5- or 6-membered heteroaromatic ring consisting of carbon atoms and from 1 to 3 heteroatoms selected from N, O and S;

R² and R³ are each independently hydrogen or C₁-C₄ alkyl; or R² and R³ together with the carbon to which they are attached form C₃-C₆ cycloalkyl;

R⁴ is C₁-C₆ alkyl, C₃-C₆ cycloalkyl, aryl, substituted aryl, heteroaryl, or substituted heteroaryl; wherein each of the substituents on substituted aryl is independently halogen, hydroxy, C₁-C₆ alkyl, C₂-C₆ alkenyl, C₂-C₆ alkynyl, fluorinated C₁-C₆ alkyl, C₁-C₆ alkoxy, or heteroaryl; and

each of the substituents on substituted heteroaryl is independently halogen, hydroxy, cyano, C₁-C₆ alkyl, C₂-C₆ alkenyl, C₂-C₆ alkynyl, fluorinated C₁-C₆ alkyl, C₁-C₆ alkoxy, or aryl;

R⁵ is carbocyclic, substituted carbocyclic, heterocyclic or substituted heterocyclic, wherein each of the substituents on substituted carbocyclic or substituted heterocyclic is independently halogen, hydroxy, C₁-C₆ alkyl, C₂-C₆ alkenyl, C₂-C₆ alkynyl, fluorinated C₁-C₆ alkyl, or C₁-C₆ alkoxy;

R⁶ is fluorinated C₁-C₆ alkyl; and

R^{a} and R^{b} are each independently hydrogen or C₁-C₄ alkyl; or R^{a} and R^{b} together with the nitrogen to which they are attached form C₃-C₆ azacycloalkyl.

The present invention further includes methods of inhibiting HIV protease, methods of treating AIDS, methods of delaying the onset of AIDS, methods of preventing infection by HIV, and methods of treating infection by HIV, wherein the methods involve administration, especially oral administration, of a therapeutically effective amount of the above-described composition of the invention, optionally in combination with other agents useful in the treatment of HIV infection and/or AIDS, to a subject in need thereof. The present invention also includes methods for making compositions of the present invention.

These and other embodiments, aspects and features of the present invention are either further described in or will be apparent from the ensuing description, examples, and appended claims.

### DETAILED DESCRIPTION OF THE INVENTION

The present invention includes complexes of a cation exchange resin with a compound of Formula (I) as defined above, and alternatively referred to herein simply as “Compound I”.

A first embodiment of the present invention is a composition which comprises a cation exchange resin complexed with a compound of Formula (I) or a pharmaceutically acceptable salt thereof, wherein:

R¹ is C₁-C₆ alkyl, C₃-C₆ cycloalkyl, aryl, substituted aryl, heteroaryl, or substituted heteroaryl, wherein heteroaryl is (i) a 5- or 6-membered aromatic ring consisting of carbon atoms and from 1 to 3 heteroatoms selected from N, S and O or (ii) an 8- to 10-membered bicyclic ring system consisting of carbon atoms and from 1 to 3 heteroatoms selected from N, S and O, wherein at least one of the rings in the bicyclic system is an aromatic ring; wherein

(i) each of the substituents on substituted aryl is independently

(a) halogen,

(b) cyano,

(c) hydroxy,

(d) C₁-C₆ alkyl,

(e) C₂-C₆ alkenyl,

(f) C₂-C₆ alkynyl,

(g) fluorinated C₁-C₆ alkyl,

(h) C₁-C₆ alkoxy,

(i) fluorinated C₁-C₆ alkoxy,

(j) S-(C₁-C₆ alkyl),

(k) heterocycle, or

(l) heterocycle substituted with one or more substituents independently selected from halogen, cyano, hydroxy, C₁-C₆ alkyl, C₂-C₆ alkenyl, C₂-C₆ alkynyl, fluorinated C₁-C₆ alkyl, C₁-C₆ alkoxy, fluorinated C₁-C₆ alkoxy, S-(C₁-C₆ alkyl), and NR^{a}R^{b}; and

(ii) each of the substituents on substituted heteroaryl is independently

(a) halogen,

(b) cyano,

(c) hydroxy,

(d) NR^{a}R^{b},

(e) C₁-C₆ alkyl,

(f) C₂-C₆ alkenyl,

(g) C₂-C₆ alkynyl,

(h) fluorinated C₁-C₆ alkyl,

(i) C₁-C₆ alkoxy,

(j) fluorinated C₁-C₆ alkoxy,

(k) S-(C₁-C₆ alkyl),

(l) phenyl,

(m) phenyl substituted with one or more substituents independently selected from halogen, cyano, hydroxy, C₁-C₆ alkyl, C₂-C₆ alkenyl, C₂-C₆ alkynyl, fluorinated C₁-C₆ alkyl, C₁-C₆ alkoxy, fluorinated C₁-C₆ alkoxy, and S-(C₁-C₆ alkyl),

(l) heterocycle, or

(m) heterocycle substituted with one or more substituents independently selected from halogen, cyano, hydroxy, C₁-C₆ alkyl, C₂-C₆ alkenyl, C₂-C₆ alkynyl, fluorinated C₁-C₆ alkyl, C₁-C₆ alkoxy, fluorinated C₁-C₆ alkoxy, S-(C₁-C₆ alkyl), NR^{a}R^{b}, and a 5- or 6-membered heteroaromatic ring consisting of carbon atoms and from 1 to 3 heteroatoms selected from N, O and S;

and all other variables are as originally defined above.

A second embodiment of the present invention is a composition which comprises a cation exchange resin complexed with a compound of Formula (I) or a pharmaceutically acceptable salt thereof, wherein:

R⁴ is

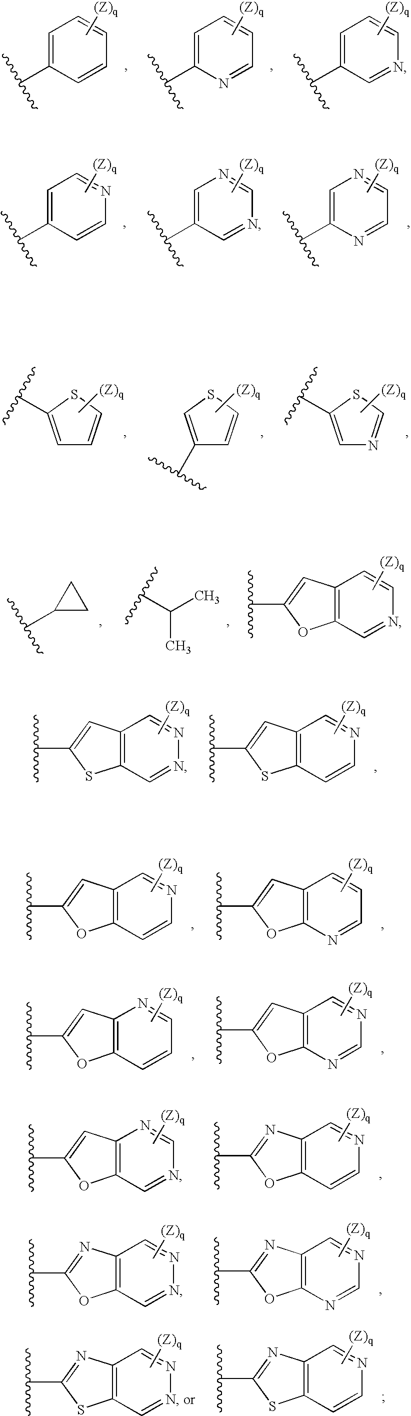

each Z is independently hydrogen, halogen, hydroxy, cyano, C₁-C₆ alkyl, C₁-C₆ fluroinated alkyl, or C₁-C₆ alkoxy;

q is an integer from 0 to 2;

and all other variables are as originally defined above or as defined in the first embodiment.

A third embodiment of the present invention is a composition which comprises a cation exchange resin complexed with a compound of Formula (I) or a pharmaceutically acceptable salt thereof, wherein:

R⁵ is carbocyclic, substituted carbocyclic, heterocyclic or substituted heterocyclic, wherein carbocyclic is cyclopentyl, indanyl, or tetralin, and heterocyclic is chroman, thiochroman, or dioxoisothiochroman; wherein each of the substituents on substituted carbocyclic or substituted heterocyclic is independently halogen, hydroxy, C₁-C₆ alkyl, fluorinated C₁-C₆ alkyl, or C₁-C₆ alkoxy;

and all other variables are as originally defined above or as defined in either the first embodiment or the second embodiment.

A fourth embodiment of the present invention is a composition which comprises a cation exchange resin complexed with a compound of Formula (I) or a pharmaceutically acceptable salt thereof, wherein:

R⁶ is

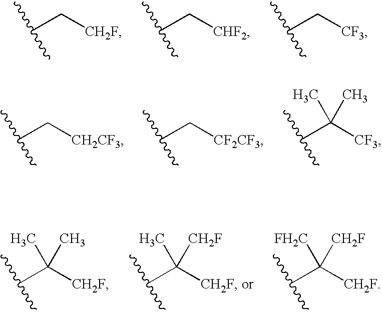

and all other variables are as originally defined above or as defined in any one of the foregoing embodiments.

A fifth embodiment of the present invention is a composition which comprises a cation exchange resin complexed with a compound of Formula (I) or a pharmaceutically acceptable salt thereof, wherein:

R¹ is

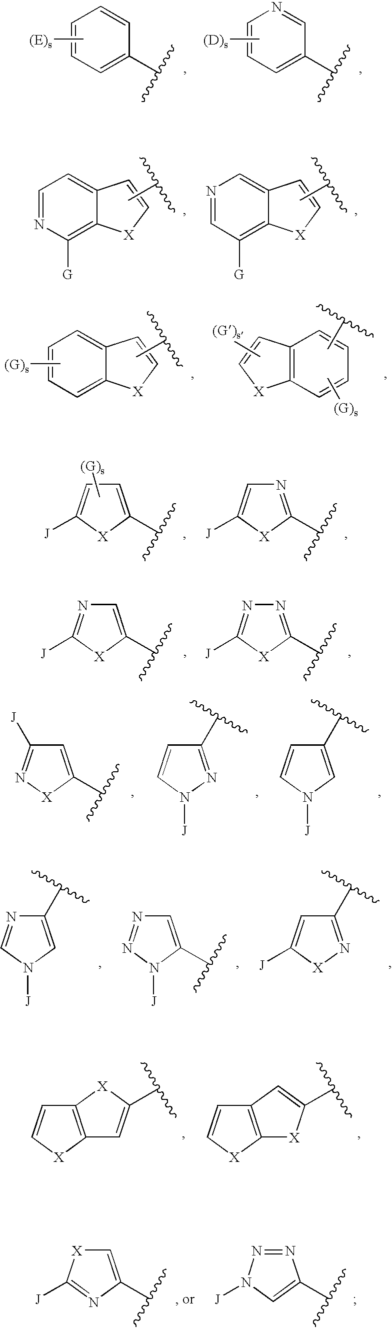

wherein:

each D is independently hydrogen, halogen, cyano, hydroxy, NR^{a}R^{b}, C₁-C₄ alkyl, C₁-C₄ alkoxy, fluorinated C₁-C₄ alkoxy, S-(C₁-C₄ alkyl), phenyl, substituted phenyl, heterocycle, or substituted heterocycle; wherein substituted phenyl is phenyl with one or more subsituents independently selected from halogen, hydroxy, C₁-C₄ alkyl, and C₁-C₄ alkoxy; and wherein substituted heterocycle is heterocycle with one or more substituents independently selected from halogen, hydroxy, C₁-C₄ alkyl, C₁-C₄ alkoxy, fluorinated C₁-C₄ alkoxy, and S-(C₁-C₄ alkyl);

each E is independently hydrogen, halogen, cyano, hydroxy, C₁-C₄ alkyl, C₁-C₄ alkoxy, heterocycle, or substituted heterocycle;

G and G′ are each independently selected from hydrogen, halogen, cyano, hydroxy, C₁-C₄ alkyl, fluorinated C₁-C₄ alkyl, and C₁-C₄ alkoxy;

J is

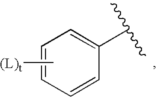

heterocycle, or substituted heterocycle;

each L is independently hydrogen, halogen, cyano, hydroxy, C₁-C₄ alkyl, fluorinated C₁-C₄ alkyl, or C₁-C₄ alkoxy;

X is O or S;

heterocycle in each of D, E and J is independently

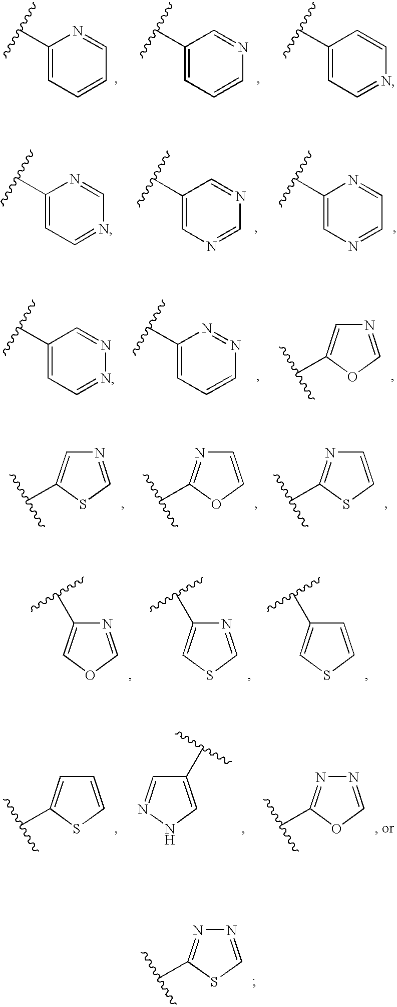

substituted heterocycle in each of E and J is independently heterocycle as defined above with one or more substituents independently selected from halogen, hydroxy, cyano, C₁-C₄ alkyl, fluorinated C₁-C₄ alkyl, C₁-C₄ alkoxy, fluorinated C₁-C₄ alkoxy, S-(C₁-C₄ alkyl), NR^{a}R^{b}, thiazolyl, oxazolyl, imidazolyl, pyrazolyl, triazolyl, pyrrolyl, isoxazolyl, and isothiazolyl; or is

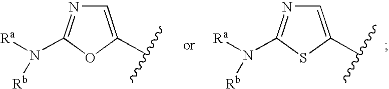

s, s′, and t are each independently integers from 0 to 2;

and all other variables are as originally defined above or as defined in the second, third, or fourth embodiments.

A sixth embodiment of the present invention is a composition which comprises a cation exchange resin complexed with a compound of Formula (I) or a pharmaceutically acceptable salt thereof, wherein:

R⁴ is

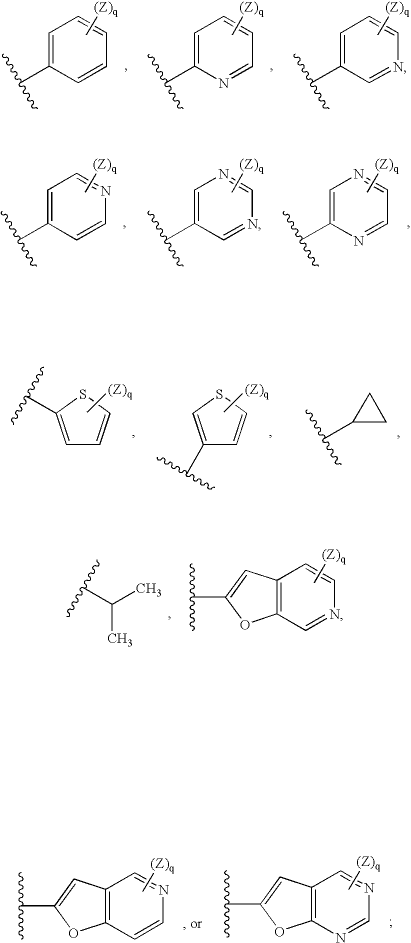

wherein:

each Z is independently hydrogen, halogen, cyano, C₁-C₆ alkyl, or C₁-C₆ alkoxy;

q is an integer from 0 to 2;

and all other variables are as originally defined above or as defined in the first, third, fourth, or fifth embodiments.

A seventh embodiment of the present invention is a composition which comprises a cation exchange resin complexed with a compound of Formula (I) or a pharmaceutically acceptable salt thereof, wherein:

R⁵ is

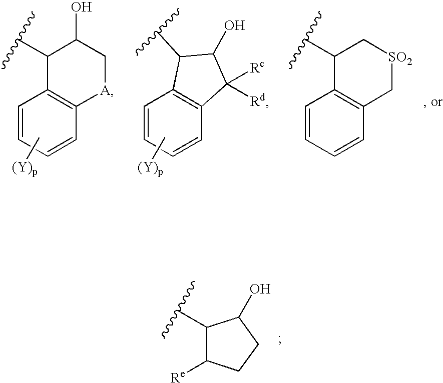

wherein:

A is CR^{c}R^{d}, O, or S;

each Y is independently hydrogen, halogen, C₁-C₆ alkyl, fluorinated C₁-C₆ alkyl, or C₁-C₆ alkoxy;

R^{c} and R^{d} are each independently hydrogen or C₁-C₄ alkyl, or R^{c} and R^{d} together with the carbon to which they are attached form C₃-C₆ cycloalkyl;

R^{e} is hydrogen, C₁-C₄ alkyl, fluorinated C₁-C₄ alkyl, or phenyl; and

p is an integer from 0 to 2;

and all other variables are as originally defined above or as defined in the first, second, fourth, fifth, or sixth embodiments.

An eighth embodiment of the present invention is a composition which comprises a cation exchange resin complexed with a compound of Formula (I) or a pharmaceutically acceptable salt thereof, wherein:

R¹ is

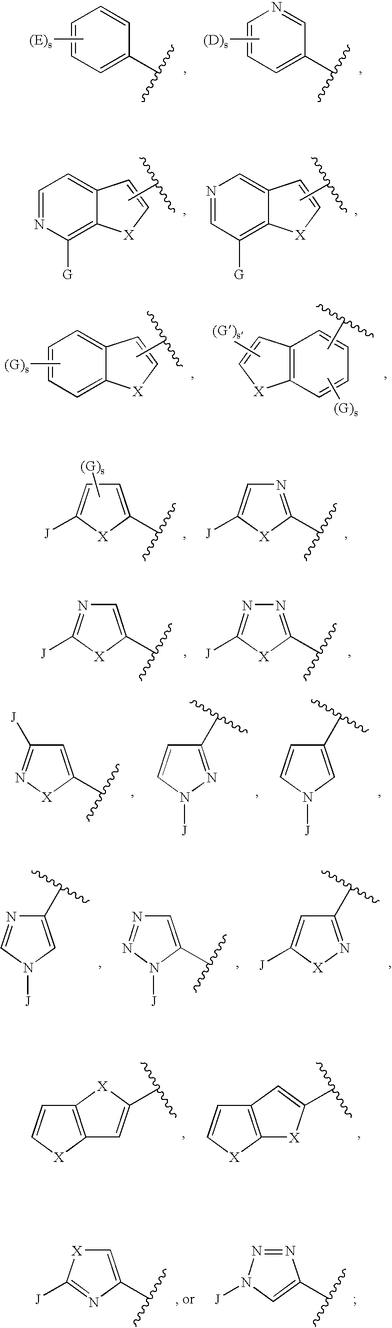

wherein:

each D is independently hydrogen, halogen, cyano, hydroxy, NR^{a}R^{b}, C₁-C₄ alkyl, C₁-C₄ alkoxy, fluorinated C₁-C₄ alkoxy, S-(C₁-C₄ alkyl), phenyl, substituted phenyl, heterocycle, or substituted heterocycle; wherein substituted phenyl is phenyl with one or more subsituents independently selected from halogen, hydroxy, C₁-C₄ alkyl, and C₁-C₄ alkoxy; and wherein substituted heterocycle is heterocycle with one or more substituents independently selected from halogen, hydroxy, C₁-C₄ alkyl, C₁-C₄ alkoxy, fluorinated C₁-C₄ alkoxy, and S-(C₁-C₄ alkyl);

each E is independently hydrogen, halogen, cyano, hydroxy, C₁-C₄ alkyl, C₁-C₄ alkoxy, heterocycle, or substituted heterocycle;

G and G′ are each independently selected from hydrogen, halogen, cyano, hydroxy, C₁-C₄ alkyl, fluorinated C₁-C₄ alkyl, and C₁-C₄ alkoxy;

J is

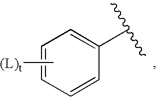

heterocycle, or substituted heterocycle;

each L is independently hydrogen, halogen, cyano, hydroxy, C₁-C₄ alkyl, fluorinated C₁-C₄ alkyl, or C₁-C₄ alkoxy;

X is O or S;

heterocycle in each of D, E and J is independently

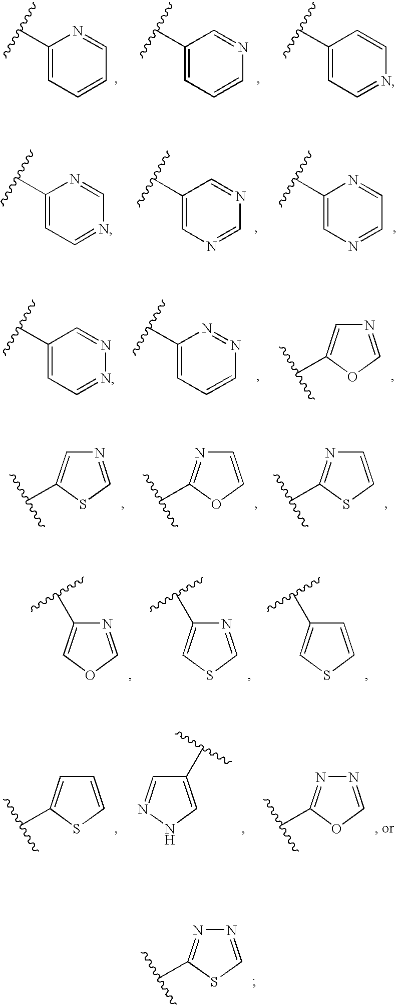

substituted heterocycle in each of E and J is independently heterocycle as defined above with one or more substituents independently selected from halogen, hydroxy, cyano, C₁-C₄ alkyl, fluorinated C₁-C₄ alkyl, C₁-C₄ alkoxy, fluorinated C₁-C₄ alkoxy, S-(C₁-C₄ alkyl), NR^{a}R^{b}, thiazolyl, oxazolyl, imidazolyl, pyrazolyl, triazolyl, pyrrolyl, isoxazolyl, and isothiazolyl; or is

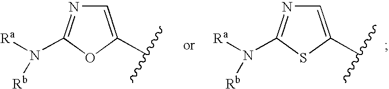

s, s′, and t are each independently integers from 0 to 2;

R⁴ is

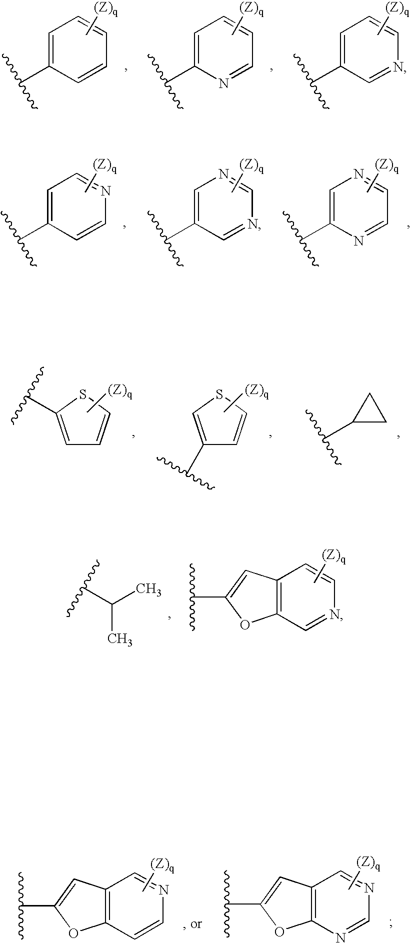

wherein:

each Z is independently hydrogen, halogen, cyano, C₁-C₆ alkyl, or C₁-C₆ alkoxy;

q is an integer from 0 to 2;

R⁵ is

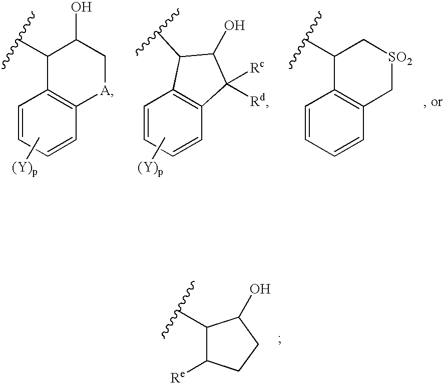

wherein:

A is CR^{c}R^{d}, O, or S;

each Y is independently hydrogen, halogen, C₁-C₆ alkyl, fluorinated C₁-C₆ alkyl, or C₁-C₆ alkoxy;

R^{c} and R^{d} are each independently hydrogen or C₁-C₄ alkyl, or R^{c} and R^{d} together with the carbon to which they are attached form C₃-C₆ cycloalkyl;

R^{e} is hydrogen, C₁-C₄ alkyl, fluorinated C₁-C₄ alkyl, or phenyl; and

p is an integer from 0 to 2; and

R⁶ is

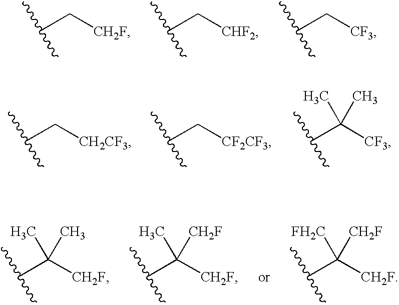

and all other variables are as originally defined above.

A ninth embodiment of the present invention is a composition which comprises a cation exchange resin complexed with a compound of Formula (I) or a pharmaceutically acceptable salt thereof, wherein:

R¹ is

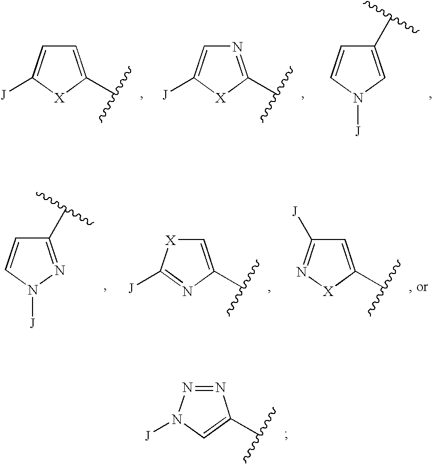

wherein:

J is

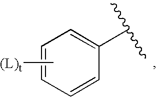

heterocycle, or substituted heterocycle;

each L is independently hydrogen, halogen, cyano, hydroxy, C₁-C₄ alkyl, fluorinated C₁-C₄ alkyl, or C₁-C₄ alkoxy;

t is an integer equal to 0, 1 or 2;

heterocycle is

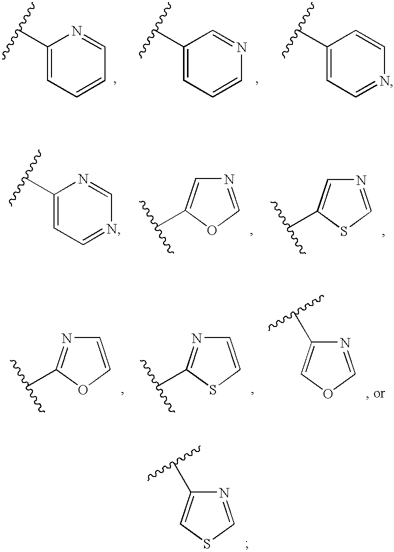

substituted heterocycle is heterocycle as defined above having one or more substituents independently selected from halogen, C₁-C₄ alkoxy, C₁-C₄ alkyl, fluorinated C₁-C₄ alkoxy, fluorinated C₁-C₄ alkyl, —S—CH₃, 13 N(CH₃)₂, thiazolyl, and oxazolyl;

X is O or S;

and all other variables are as originally defined or as defined in the second, third, fourth, sixth, seventh or ninth embodiments.

In an aspect of the preceding embodiment, R¹ is

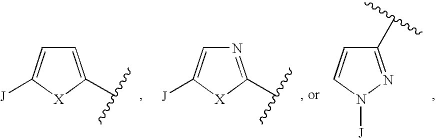

wherein:

J is

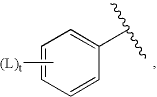

heterocycle, or substituted heterocycle;

each L is independently hydrogen, halogen, cyano, hydroxy, C₁-C₄ alkyl, fluorinated C₁-C₄ alkyl, or C₁-C₄ alkoxy;

t is an integer equal to 0, 1 or 2;

heterocycle is

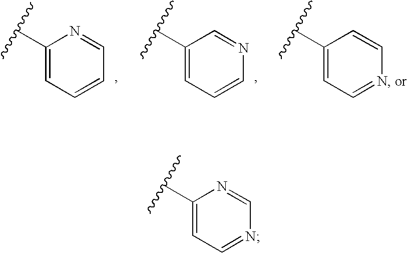

substituted heterocycle is heterocycle as defined above having one or more substituents independently selected from halogen, C₁-C₄ alkoxy, C₁-C₄ alkyl, fluorinated C₁-C₄ alkoxy, fluorinated C₁-C₄ alkyl, —S—CH₃, —N(CH₃)₂, thiazolyl, and oxazolyl; and

X is O or S.

A tenth embodiment of the present invention is a composition which comprises a cation exchange resin complexed with a compound of Formula (I) or a pharmaceutically acceptable salt thereof, wherein:

R⁴ is

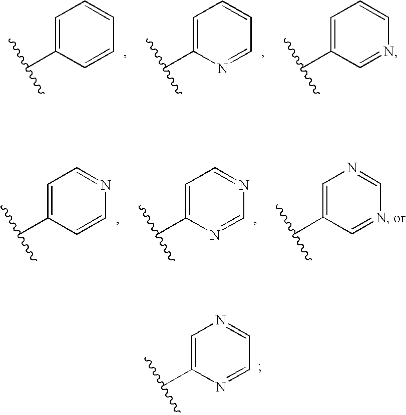

and all other variables are as originally defined or as defined in the first, third, fourth, fifth, seventh, eighth, or ninth embodiments.

In an aspect of the preceding embodiment, R⁴ is

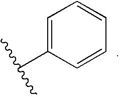

An eleventh embodiment of the present invention is a composition which comprises a cation exchange resin complexed with a compound of Formula (I) or a pharmaceutically acceptable salt thereof, wherein:

R⁵ is

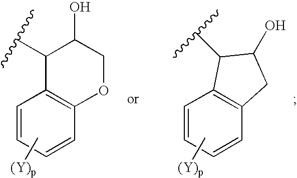

wherein:

each Y is independently hydrogen, halogen, C₁-C₆ alkyl, fluorinated C₁-C₆ alkyl, or C₁-C₄ alkoxy; and

p is an integer from 0 to 2;

and all other variables are as originally defined or as defined in the first, second, fourth, fifth, sixth, eighth, ninth, or tenth embodiments.

In an aspect of the preceding embodiment, R⁵ is

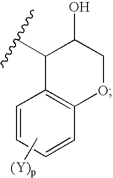

wherein:

each Y is independently hydrogen, halogen, C₁-C₆ alkyl, fluorinated C₁-C₆ alkyl, or C₁-C₄ alkoxy; and

p is an integer from 0 to 2.

A twelfth embodiment of the present invention is a composition which comprises a cation exchange resin complexed with a compound of Formula (I) or a pharmaceutically acceptable salt thereof, wherein:

R⁶ is

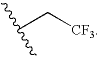

and all other variables are as originally defined or as defined in the first, second, third, fifth, sixth, seventh, eighth, ninth, tenth or eleventh embodiments.

A thirteenth embodiment of the present invention is a composition which comprises a cation exchange resin complexed with a compound of Formula (I) or a pharmaceutically acceptable salt thereof, wherein:

R¹ is

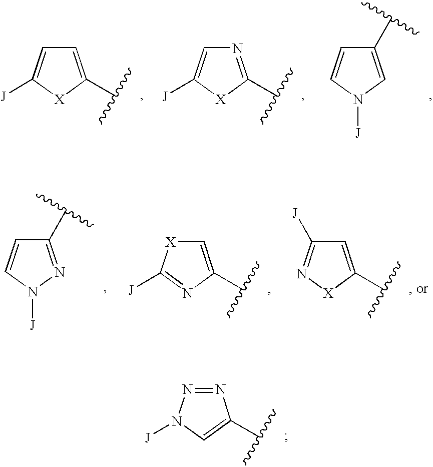

wherein:

J is

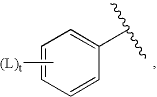

heterocycle, or substituted heterocycle;

each L is independently hydrogen, halogen, cyano, hydroxy, C₁-C₄ alkyl, fluorinated C₁-C₄ alkyl, or C₁-C₄ alkoxy;

t is an integer equal to 0, 1 or 2;

heterocycle is

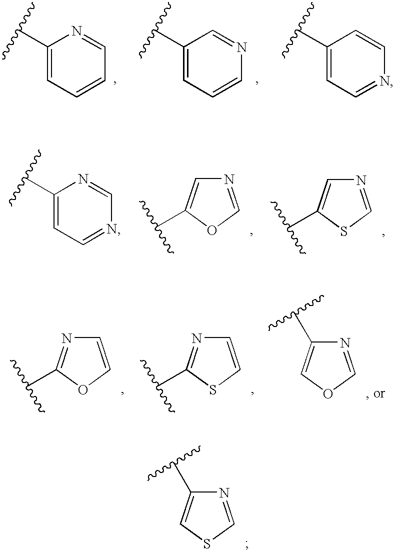

substituted heterocycle is heterocycle as defined above having one or more substituents independently selected from halogen, C₁-C₄ alkoxy, C₁-C₄ alkyl, fluorinated C₁-C₄ alkoxy, fluorinated C₁-C₄ alkyl, —S—CH₃, —N(CH₃)₂, thiazolyl, and oxazolyl; and

X is O or S;

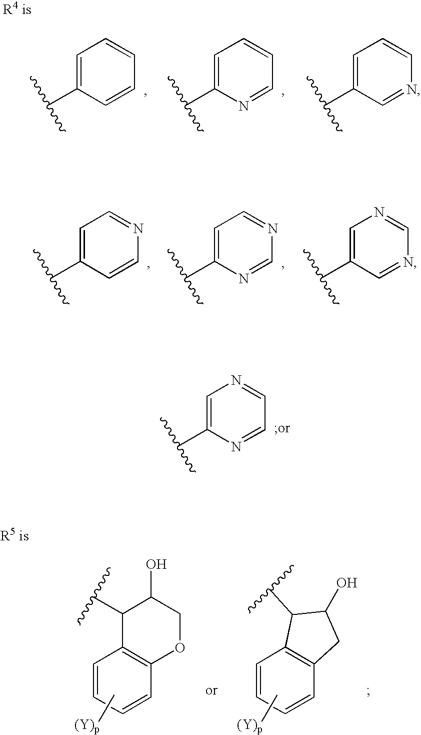

wherein:

each Y is independently hydrogen, halogen, C₁-C₆ alkyl, fluorinated C₁-C₆ alkyl, or C₁-C₄ alkoxy; and

p is an integer from 0 to 2; and

R⁶ is

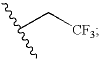

and all other variables are as originally defined.

A fourteenth embodiment of the present invention is a composition which comprises a cation exchange resin complexed with a compound of Formula (I) or a pharmaceutically acceptable salt thereof, wherein:

R¹ is

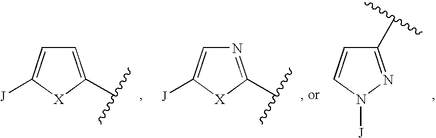

wherein:

J is

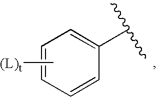

heterocycle, or substituted heterocycle;

each L is independently hydrogen, halogen, cyano, hydroxy, C₁-C₄ alkyl, fluorinated C₁-C₄ alkyl, or C₁-C₄ alkoxy;

t is an integer equal to 0, 1 or 2;

heterocycle is

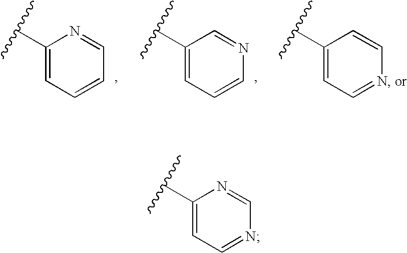

substituted heterocycle is heterocycle as defined above having one or more substituents independently selected from halogen, C₁-C₄ alkoxy, C₁-C₄ alkyl, fluorinated C₁-C₄ alkoxy, fluorinated C₁-C₄ alkyl, —S—CH₃, —N(CH₃)₂, thiazolyl, and oxazolyl; and

X is O or S;

R⁴ is

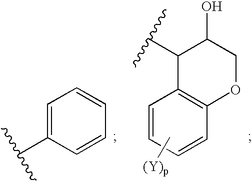

R⁵ is

wherein:

each Y is independently hydrogen, halogen, C₁-C₆ alkyl, fluorinated C₁-C₆ alkyl, or C₁-C₄ alkoxy; and

p is an integer from 0 to 2;

R⁶ is

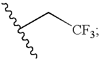

and all other variables are as originally defined.

In an aspect of the preceding embodiment,

each L is independently hydrogen, chlorine, or fluorine;

each Y is independently hydrogen, chlorine, or fluorine; and

each of the substituents on substituted heterocycle is independently chlorine, fluorine, methoxy, ethoxy, —OCF₃, —OCHF₂, methyl, ethyl, n-propyl, —S—CH₃, —N(CH₃)₂, and thiazolyl.

A fifteenth embodiment of the present invention is a composition which comprises a cation exchange resin complexed with a compound of Formula (I) or a pharmaceutically acceptable salt thereof, wherein:

R² and R³ are each independently hydrogen or methyl;

and all other variables are as originally defined or a defined in any of the foreoging embodiments or aspects.

Suitable compositions of the present invention include those which comprise a cation exchange resin complexed with a compound selected from the group consisting of:

(αR,γS,2S)-N-((3S,4S)-3,4-dihydro-3-hydroxy-2H-1-benzopyran-4-yl)-4-(1-furo[3,2-c]pyridin-2-yl-1-methylethyl)-γ-hydroxy-α-(phenylmethyl)-2-[[(2,2,2-trifluoroethyl)amino]carbonyl]-1-piperazinepentanamide;

(αR,γS,2S)-N-((3S,4S)-3,4-dihydro-3-hydroxy-2H-1-benzopyran-4-yl)-γ-hydroxy-4-[(5-phenyl-2-furanyl)methyl]-α-(4-pyridinylmethyl)-2-[[(2,2,2-trifluoroethyl)amino]-carbonyl]-1-piperazinepentanamide;

(αR,γS, 2S)-N-((3S,4S)-3,4-dihydro-3-hydroxy-2H-1-benzopyran-4-yl)-γ-hydroxy-4-[1-methyl-1-(1-phenyl-1H-pyrazol-3-yl)ethyl]-α-(3-pyridinylmethyl)-2-[[(2,2,2-trifluoroethyl)amino]carbonyl]-1-piperazinepentanamide;

(αR,γS,2S)-N-((3S,4S)-3,4-dihydro-3-hydroxy-2H-1-benzopyran-4-yl)-γ-hydroxy-α-(phenylmethyl)-4-[[5-(2-pyridinyl)-2-furanyl]methyl]-2-[[(2,2,2-trifluoroethyl)-amino]carbony]-1-piperazinepentanamide;

(αR,γS,2S)-4-[[5-(5-chloro-2-pyridinyl)-2-furanyl]methyl]-N-((3S,4S)-3,4-dihydro-3-hydroxy-2H-1-benzopyran-4-yl)-γ-hydroxy-α-(phenylmethyl)-2-[[(2,2,2-trifluoroethyl)amino]carbonyl]-1-piperazinepentanamide;

(αR,γS,2S)-N-((3S,4S)-3,4-dihydro-3-hydroxy-2H-1-benzopyran-4-yl)-γ-hydroxy-4-[1-methyl-1-[5-(3-pyridinyl)-2-oxazolyl]ethyl]-α-(phenylmethyl)-2-[[(2,2,2-trifluoroethyl)amino]carbonyl]-piperazinepentanamide;

(αR,γS,2S)-N-[(3S,4S)-3,4-dihydro-3-hydroxy-2H-1-benzopyran-4-yl]-γ-hydroxy-4-[[1-[5-(5-methoxy-3-pyridinyl)-2-oxazolyl]-1-methylethyl]-α-(phenylmethyl)-2-[[(2,2,2-trifluoroethyl)amino]carbonyl]-1-piperazinepentanamide;

(αR,γS,2S)-N-[(3S,4S)-3,4-dihydro-3-hydroxy-2H-1-benzopyran-4-yl]-γ-hydroxy-4-[1-[5-(5-fluoro-3-pyridinyl)-2-oxazolyl]-1-methylethyl]-α-(phenylmethyl)-2-[[(2,2,2-trifluoroethyl)amino]carbonyl]-1-piperazinepentanamide;

(αR,γS,2S)-N-[(3S,4S)-3,4-dihydro-3-hydroxy-2H-1-benzopyran-4-yl]-γ-hydroxy-4-[1-[1-(5-fluoro-3-pyridinyl)-1H-pyrazol-3-yl]-1-methylethyl]-α-(phenylmethyl)-2-[[(2,2,2-trifluoroethyl)amino]carbonyl]-1-piperazinepentanamide;

(αS,γS,2S)-4-[1-[5-(4-chlorophenyl)-2-oxazolyl]-1-methylethyl]-N-[(3S,4S)-3,4-dihydro-3-hydroxy-2H-1-benzopyran-4-yl]-α-(furo[2,3-c]pyridin-2-ylmethyl)-γ-hydroxy-2-[[(2,2,2-trifluoroethyl)amino]carbonyl]-1-piperazinepentanamide;

(αS,γS,2S)-4-[1-[5-(4-fluorophenyl)-2-oxazolyl]-1-methylethyl]-N-[(3S,4S)-3,4-dihydro-3-hydroxy-2H-1-benzopyran-4-yl]-α-(furo [2,3-c]pyridin-2-ylmethyl)-γ-hydroxy-2-[[(2,2,2-trifluoroethyl)amino]carbonyl]-1-piperazinepentanamide;

(αS,γS,2S)-4-[1-[5-(4-chlorophenyl)-2-oxazolyl]-1-methylethyl]-N-[(3S,4S)-3,4-dihydro-3-hydroxy-2H-1-benzopyran-4-yl]-α-(furo[2,3-c]pyridin-3-ylmethyl)-γ-hydroxy-2-[[(2,2,2-trifluoroethyl)amino]carbonyl]-1-piperazinepentanamide;

(αS,γS,2S)-4-[1-[5-(4-fluorophenyl)-2-oxazolyl]-1-methylethyl]-N-[(3S,4S)-3,4-dihydro-3-hydroxy-2H-1-benzopyran-4-yl]-α-(furo[2,3-c]pyridin-3-ylmethyl)-γ-hydroxy-2-[[(2,2,2-trifluoroethyl)amino]carbonyl]-1-piperazinepentanamide;

(αS,γS,2S)-N-[(3S,4S)-3,4-dihydro-3-hydroxy-2H-1-benzopyran-4-yl]-4-[1-[5-(4-fluorophenyl)-2-oxazolyl]-1-methylethyl]-α-(furo[2,3-d]pyrimidin-6-ylmethyl)-γ-hydroxy-2-[[(2,2,2-trifluoroethyl)amino]carbonyl]-1-piperazinepentanamide;

and pharmaceutically acceptable salts thereof.

A preferred composition of the present invention is a composition which comprises a cation exchange resin complexed with Compound A or a pharmaceutically acceptable salt thereof.

Another embodiment of the present invention is a composition which comprises a cation exchange resin which is an acidic sulfonic acid resin or an acidic carboxylic acid resin complexed with a compound of Formula (I) or a pharmaceutically acceptable salt thereof, wherein the compound is as originally defined or as defined in any of the foregoing embodiments or aspects. In an aspect of the preceding embodiment, the cation exchange resin is a strongly acidic resin. In another aspect of the preceding embodiment, the cation exchange resin is an acidic sulfonic acid resin. In still another aspect of the preceding embodiment, the cation exchange resin is Amberlite IRP-69.

Still another embodiment of the present invention is a composition which comprises the cation exchange resin Amberlite IRP-69 complexed with Compound A or a pharmaceutically acceptable salt thereof.

Yet another embodiment of the present invention is a composition as originally defined above or as defined in any of the foregoing embodiments or aspects, wherein the composition further comprises a capsule containing the complex (i.e., the complex is encapsulated).

Other embodiments of the present invention include the following:

(a) A method of inhibiting HIV protease in a subject in need thereof which comprises administering to the subject a therapeutically effective amount of a composition comprising a cation exchange resin complexed with a compound of Formula (I).

(b) A method of preventing or treating infection by HIV in a subject in need thereof which comprises administering to the subject a therapeutically effective amount of a composition comprising a cation exchange resin complexed with a compound of Formula (I).

(c) A method of treating AIDS in a subject in need thereof which comprises administering to the subject a therapeutically effective amount of a composition comprising a cation exchange resin complexed with a compound of Formula (I).

(d) A method of delaying the onset of AIDS in a subject in need thereof which comprises administering to the subject a therapeutically effective amount of a composition comprising a cation exchange resin complexed with a compound of Formula (I).

(e) The method of (a) or (b) or (c) or (d), wherein the composition as defined therein is administered in combination with a therapeutically effective amount of at least one HIV/AIDS treatment agent selected from the group consisting of EV/AIDS antiviral agents, immunomodulators, and anti-infective agents.

(f) The method of (a) or (b) or (c) or (d), wherein the composition as defined therein is administered in combination with a therapeutically effective amount of at least one antiviral agent selected from the group consisting of non-nucleoside HIV reverse transcriptase inhibitors and nucleoside HIV reverse transcriptase inhibitors.

(g) The method of (a) or (b) or (c) or (d), wherein the composition as defined therein is administered in combination with a therapeutically effective amount of an antiviral agent which is a CCR5 receptor antagnoist.

(h) The method of (a) or (b) or (c) or (d), wherein the composition as defined therein is administered in combination with a therapeutically effective amount of an antiviral agent which is an HIV integrase inhibitor.

Additional embodiments of the invention include the methods set forth in (a)-(h) above, wherein Compound I complexed with the cation exchange resin is a compound as defined in any one of the embodiments, aspects or features set forth above.

Still other embodiments of the present invention include the following:

(i) A combination comprising (1) a therapeutically effective amount of a composition which comprises a cation exchange resin complexed with a compound of Formula (I) and (2) a therapeutically effective amount of at least one HIV/AIDS treatment agent selected from the group consisting of HIV/AIDS antiviral agents, immunomodulators, and anti-infective agents.

(j) The combination of (i) wherein the HIV/AIDS treatment agent is an HIV/AIDS antiviral agent selected from the group consisting of non-nucleoside HIV reverse transcriptase inhibitors and nucleoside HIV reverse transcriptase inhibitors.

(k) The combination of (j), further comprising a therapeutically effective amount of an additional HIV protease inhibitor.

(l) The combination of (i) wherein the HIV/AIDS treatment agent is an antiviral agent which is a CCR5 receptor antagonist.

(m) The combination of (i) wherein the HIV/AIDS treatment agent is an antiviral agent which is an HIV integrase inhibitor.

(n) A combination comprising (1) a therapeutically effective amount of a composition which comprises a cation exchange resin complexed with a compound of Formula (I) and (2) and a cytochrome P450 monooxygenase inhibitor (e.g., indinavir or ritonavir or a pharmaceutically acceptable salt thereof) in an amount effective to improve the pharmacokinetics of Compound I.

Additional embodiments of the invention include the combinations set forth in (i)-(n) above, wherein Compound I complexed with the cation exchange resin is a compound as defined in any one of the embodiments, aspects or features set forth above.

The present invention also includes a process for preparing a composition comprising a cation exchange resin complexed with a compound of Formula (I), which comprises:

(A) contacting a cation exchange resin with a compound of Formula (I) in an aqueous or polar organic medium under conditions and for a time sufficient for the compound to form a complex with the resin; and

(B) recovering the complex.

In an embodiment of the process, the cation exchange resin is an acidic sulfonic acid resin.

Additional embodiments of the process include the process in which the compound of Formula (I) contacted with the resin in Step A is a compound as defined in any one of the embodiments, aspects or features set forth above.

The present invention also includes a composition prepared by contacting a cation exchange resin (e.g., an acidic sulfonic acid resin) with a compound of Formula (I) in an aqueous or polar organic medium under conditions and for a time sufficient for the compound to form a complex with the resin.

The present invention further includes a complex formed by contacting a cation exchange resin with a compound of Formula (I) as originally defined above or as defined in any of the embodiments, aspects or features as set forth above.

The present invention also includes a cation exchange resin-Compound I complex as defined and described above for use in (a) inhibiting HIV protease, (b) preventing or treating infection by HIV, or (c) treating or delaying the onset of AIDS or ARC. The present invention further includes use of a resin-Compound I complex as defined and described above as a medicament for (a) inhibiting HIV protease, (b) preventing or treating infection by HIV, or (c) treating or delaying the onset of AIDS or ARC. The present invention also includes use of a resin-Compound I complex as defined and described above in the preparation of a medicament for (a) inhibiting HIV protease, (b) preventing or treating infection by HIV, or (c) treating or delaying the onset of AIDS or ARC.

As used herein, the term “C₁-C₆ alkyl” refers to a linear or branched chain alkyl group having from 1 to 6 carbon atoms, and is selected from the hexyl alkyl and pentyl alkyl isomers, n-, iso-, sec- and t-butyl, n- and isopropyl, ethyl and methyl. “C₁-C₄ alkyl” refers to a linear or branched chain alkyl group having from 1 to 4 carbon atoms, and is selected from n-, iso-, sec- and t-butyl, n- and isopropyl, ethyl and methyl.

The term “C₂-C₆ alkenyl” refers to a linear or branched chain alkenyl group having from 2 to 6 carbon atoms, and is selected from the hexyl alkenyl and pentyl alkenyl isomers, 1-, 2- and 3-butenyl, 1- and 2-isobutenyl, 1- and 2-propenyl, and ethenyl. “C₂-C₄ alkenyl” has an analogous definition.

The term “C₂-C₆ alkynyl” refers to a linear or branched chain alkynyl group having from 2 to 6 carbon atoms, and is selected from the hexyl alkynyl and pentyl alkynyl isomers, 1-, 2- and 3-butynyl, 1- and 2-propynyl, and ethynyl. “C₂-C₄ alkynyl” has an analogous definition.

The term “C₁-C₆ alkoxy” means an —O-alkyl group wherein alkyl is C₁ to C₆ alkyl as defined above. “C₁-C₄ alkoxy” has an analogous meaning; i.e., it is an alkoxy group selected from methoxy, ethoxy, n-propoxy, isopropoxy, n-butoxy, isobutoxy, tert-butoxy, and sec-butoxy. Similarly, “C₁-C₃ alkoxy” is selected from methoxy, ethoxy, n-propoxy, and isopropoxy.

The term “C₃-C₆ cycloalkyl” refers to a cyclic ring selected from cyclopropyl, cyclobutyl, cyclopentyl, and cyclohexyl. “C₃-C₅ cycloalkyl” has an analogous meaning.

The term “C₃-C₆ azacycloalkyl” refers to a saturated monocyclic group consisting of one nitrogen and from 3 to 6 carbon atoms, selected from azetidinyl (i.e., azacyclobutyl), pyrrolidinyl (azacyclopentyl), piperidinyl (azacyclohexyl), and hexahydroazepinyl (azacycloheptyl). “C₃-C₅ azacycloalkyl” has an analogous meaning.

The term “halogen” (which may alternatively be referred to as “halo”) refers to fluorine, chlorine, bromine and iodine (alternatively, fluoro, chloro, bromo, and iodo).

The term “fluorinated C₁-C₆ alkyl” (which may alternatively be referred to as “C₁-C₆ fluoroalkyl”) means a C₁-C₆ alkyl group as defined above with one or more fluorine substituents. The term “fluorinated C₁-C₄ alkyl” has an analogous meaning. Representative examples of suitable fluoroalkyls include the series (CH₂)₀₋₃CF₃ (i.e., trifluoromethyl, 2,2,2-trifluoroethyl, 3,3,3-trifluoro-n-propyl, etc.), 1-fluoroethyl, 2-fluoroethyl, 2,2-difluoroethyl, 3,3,3-trifluoroisopropyl, 1,1,1,3,3,3-hexafluoroisopropyl, and perfluorohexyl.

The term “fluorinated C₁-C₆ alkoxy” (which may alternatively be referred to as “C₁-C₆ fluoroalkoxy”) means a C₁-C₆ alkoxy group as defined above wherein the alkyl moiety has one or more fluorine substituents. The terms “fluorinated C₁-C₄ alkoxy” and “fluorinated C₁-C₃ alkoxy” have analogous meanings. Representative examples include the series O(CH₂)₀₋₃CF₃ (i.e., trifluoromethoxy, 2,2,2-trifluoroethoxy, 3,3,3-trifluoro-n-propoxy, etc.), 1,1,1,3,3,3-hexafluoroisopropoxy, and so forth.

The term “carbocyclic” (which may alternatively be referred to as “carbocycle”) refers to a saturated or unsaturated monocyclic ring consisting of from 5 to 7 carbon atoms or a saturated or unsaturated bicyclic ring consisting of from 7 to 10 carbon atoms. It is understood that either or both rings of the bicyclic may be saturated or unsaturated. Exemplary carbocyclics include, but are not limited to, cyclopentyl, cyclohexyl, cylcoheptyl, cyclopentenyl, cyclohexenyl, cycloheptenyl, phenyl, naphthyl, tetrahydronaphthyl (tetralin), indenyl, and indanyl.

The term “aryl” refers to aromatic mono- and poly-carbocyclic ring systems, wherein the carbocyclic rings in the polyring systems may be fused or attached to each other via a single ring carbon. Suitable aryl groups include, but are not limited to, phenyl, naphthyl, and biphenylenyl.

The term “substituted aryl” refers to an aryl group as defined above having one or more substituents (e.g., having from 1 to 5 or from 1 to 4 or from 1 to 3 substituents, or having 1 or 2 substituents, or is mono-substituted) independently selected from cyano, halo, hydroxy, C₁-C₆ alkyl, C₂-C₆ alkenyl, C₂-C₆ alkynyl, C₁-C₆ alkoxy, fluorinated C₁-C₆ alkyl, fluorinated C₁-C₆ alkoxy, heterocycle, substituted heterocycle, and the like.

The term “heterocyclic” (which may alternatively be referred to as “heterocycle”) refers to (i) a 4- to 8-membered, saturated or unsaturated monocyclic ring consisting of carbon atoms and one or more heteroatoms selected from N, O and S or (ii) a 7- to 10-membered bicyclic ring system, either ring of which is saturated or unsaturated, consisting of carbon atoms and one or more heteroatoms selected from N, O and S; and wherein the nitrogen and sulfur heteroatoms in (i) or (ii) are optionally oxidized, and the nitrogen heteroatom is optionally quaternized. The heterocyclic ring may be attached at any heteroatom or carbon atom, provided that attachment results in the creation of a stable structure. Representative examples of heterocyclic groups include azetidinyl, piperidinyl, piperazinyl, azepinyl, pyrrolyl, indazolyl, pyrrolidinyl, pyrazolyl, pyrazolidinyl, imidazolyl, imidazolidinyl, imidazolinyl, pyridyl, pyrazinyl, pyrimidinyl, pyridazinyl, oxazolyl, oxazolidinyl, triazolyl, isoxazolyl, isoxazolidinyl, morpholinyl, thiazolyl, thiadiazolyl, thiazolidinyl, isothiazolyl, quinoxazolinyl, isothiazolidinyl, methylenedioxyphenyl, quinolinyl, isoquinolinyl, benzimidazolyl, thiadazolyl, benzopyranyl, benzothiazolyl, benzoazolyl, furyl, tetrahydrofuryl, benzofuranyl, benzothiofuranyl, azabenzofuranyl, benzothiazolyl, azabenzothiazolyl, azabenzoxazolyl, tetrahydropuranyl, thiophenyl (alternatively referred to herein as “thienyl”), thienothiophenyl, benzothiophenyl, and oxadiazolyl.

The term “substituted heterocyclic” (alternatively “substituted heterocycle”) refers to a heterocyclic group as defined above having one or more substituents (e.g., having from 1 to 7 or from 1 to 6 or from 1 to 5 or from 1 to 4 or from 1 to 3 substituents, or having 2 substituents, or is mono-substituted) independently selected from cyano, halo, hydroxy, amino, C₁-C₄ alkylamino, di-(C₁-C₄ alkyl)amino, C₃-C₆ azacycloalkyl, C₁-C₆ alkyl, C₂-C₆ alkenyl, C₂-C₆ alkynyl, C₁-C₆ alkoxy, fluorinated C₁-C₆ alkyl, fluorinated C₁-C₆ alkoxy, aryl (e.g., phenyl), and the like. A substituent may be attached to either a ring carbon or ring heteroatom in the substituted heterocyclic group.

The term “heteroaryl” refers to a heterocyclic group as defined above, wherein the monocyclic ring (i) is an aromatic ring and in the bicyclic ring system (ii) at least one ring is an aromatic ring. In one aspect, heteroaryl refers to (i) a 5- or 6-membered aromatic ring consisting of carbon atoms and from 1 to 3 heteroatoms selected from N, S and O or (ii) an 8- to 10-membered bicyclic ring system consisting of carbon atoms and from 1 to 3 heteroatoms selected from N, S and O, wherein at least one of the rings in the bicyclic system is an aromatic ring.

The term “substituted heteroaryl” refers to a heteroaryl group as defined above having one or more substituents (e.g., having from 1 to 5 or from 1 to 4 or from 1 to 3 substituents, or having 1 or 2 substituents, or is mono-substituted) independently selected from cyano, halo, hydroxy, amino, C₁-C₄ alkylamino, (di-(C₁-C₄ alkyl)amino, C₃-C₆ azacycloalkyl, C₁-C₆ alkyl, C₂-C₆ alkenyl, C₂-C₆ alkynyl, C₁-C₆ alkoxy, fluorinated C₁-C₆ alkyl, fluorinated C₁-C₆ alkoxy, aryl (e.g., phenyl), substituted aryl, heterocycle, and substituted heterocycle. A substituent may be attached to either a ring carbon or ring heteroatom in the substituted heteroaryl group.

The term “substituted” includes mono- and poly-substitution by a named substituent to the extent such single and multiple substitution is chemically allowed and results in a chemically stable compound.

The symbol “ [symbol] ” in front of an open bond in the structural formula of a group marks the point of attachment of the group to the rest of the molecule.

When any variable or term occurs more than one time in any constituent or formulas set forth herein (e.g., Formula (I)), its definition on each occurrence is independent of its definition at every other occurrence. Thus, for example, if R² and R³ in Formula (I) are both designated as “C₁C₄ alkyl”, R² and R³ can represent the same or different alkyl groups embraced by the term. As another example, in an embodiment of Formula (I) in which R¹ and R⁴ are both heteroaryl, R¹ and R⁴ can be the same or different heteroaryl groups.

Combinations of substituents and/or variables are permitted only to the extent such combinations result in stable compounds.

The term “complex” and variants thereof (e.g., complexed), when used herein in reference to compounds of Formula (I) (alternatively “Compound I”) and a cation exchange resin, means that Compound I is reversibly associated with or supported by the cation exchange resin. While not wishing to be bound by theory, it is believed that the Compound I-resin complex is formed by an ionic interaction between a positively charged group (e.g., ammonium) on Compound I and a negatively charged group (e.g., sulfonate) on the resin.

The term “therapeutically effective amount” as used herein means that amount of active compound or pharmaceutical agent that elicits the biological or medicinal response in a tissue, system, animal or human that is being sought by a researcher, veterinarian, medical doctor or other clinician, which includes alleviation of the symptoms of the disease being treated and/or the prevention or delay in onset or recurrence of a pathology.

The expression “pharmaceutically acceptable” means that the salt, carrier, diluent or excipient must be compatible with the other ingredients of the formulation and not deleterious to the recipient thereof.

The term “subject” (alternatively referred to herein as “patient”) as used herein refers to an animal, typically a mammal and preferably a human, who has been the object of treatment, observation or experiment.

The term “administration” and variants thereof (e.g., “administering” a composition) in reference to the composition of the present invention mean providing the composition to the subject or individual in need of treatment. When a composition of the invention is provided in combination with one or more other active agents (e.g., HIV/AIDS antivirals), “administration” and its variants are each understood to include concurrent and time-separated (e.g., alternating) provision of the composition and other agents.

The compounds of Formula (I) employed in the complexes of the present invention are inhibitors of HIV protease, including mutant forms thereof that are resistant to known protease inhibitors. Representative compounds of Formula (I) have exhibited IC₅₀ values less than about 1 nM against the wild-type enzyme and less than about 5 nM against the mutant enzymes Q-60, K-60, and V-18 in the assay for inhibition of microbial expressed HIV protease described in International Publication No. WO 01/05230. Representative compounds of Formula (I) have also exhibited CIC₉₅ values of less than about 50 nM against the wild-type viral construct and CIC₉₅ values of less than about 125 nM against the viral constructs Q60, K-60, and V-18 in the cell spread assay described in WO 01/05230.

The cation exchange resins suitable for use in the present invention are water-insoluble and consist of a pharmacologically inert organic or inorganic matrix containing covalently bound functional groups that are anionic or capable of becoming anionic under the appropriate conditions of pH. The organic matrix may be synthetic (e.g., polymers or copolymers of acrylic acid, methacrylic acid, sulfonated styrene, sulfonated divinylbenzene), or partially synthetic (e.g., modified cellulose and dextrans). The inorganic matrix can be, for example, silica gel modified by the addition of ionic groups. The covalently bound anionic groups may be strongly acidic (e.g., sulfonate) or weakly acidic (e.g., carboxylate) groups. In general, those types of cation exchangers suitable for use in ion exchange chromatography and for such applications as deionization of water are suitable for use in preparing complexes of the present invention. Suitable cation exchangers are described in H. F. Walton, “Principles of Ion Exchange”, Chapter 12 in Chromatography, edited by E. Heftmann, Van Nostrand Reinhold Co., New York, 1975, pp. 312-343, which is incorporated herein by reference. Further description of suitable cation exchangers, their preparation and properties can be found in F. de Dardel and T. V. Arden, “Ion Exchangers”, in Ullman's Encyclopedia of Industrial Chemistry, Vol. A14, edited by B. Elvers et al., 1989, pp. 393-459, and in C. Dickert, “Ion Exchange”, in Kirk-Othmer Encyclopedia of Chemical Technology, 4^{th} edition, Vol. 14, edited by M. Howe-Grant, Wiley-Interscience, New York, 1995, pp. 737-783, both of which are incorporated herein by reference in their entireties.

The synthetic organic matrices are typically cross-linked with a cross-linking agent which is a difunctional compound capable of cross-linking the polymers comprising the organic resin (e.g., polystyrenes or polymethacrylics). Suitable cross-linking agents are well known in the art. The typical cross-linking agent is a divinyl or polyvinyl compound, such as divinylbenzene. Divinylbenzene is commonly employed, for example, to prepare cross-linked polystyrenes. The resin is suitably cross-linked to a level in the range of from about 3 to about 20 wt. % (e.g., from about about 4 to about 16 wt. %) based on the total resin. In one embodiment of the present invention, the resin is a styrene resin cross-linked to a level in the range of from about 6 to about 10 wt. % (e.g., about 8 wt. %) based on the total resin. The resins can be cross-linked with the selected cross-linking agent by means well known in the art. The preparation and properties of suitable cross-linked resins are further described in the entries from Ullman's Encyclopedia of Industrial Chemistry, Vol. A14, and from Kirk-Othmer Encyclopedia of Chemical Technology, 4^{th} edition, Vol. 14, cited in the preceding paragraph.

Representative resins useful in this invention include Amberlite IRP-69 (available from Rohm and Haas) and Dow XYS-40010.00 (available from Dow Chemical Company). Both of these resins are sulfonated polymers composed of polystyrene cross-linked with about 8% of divinylbenzene, with an ion exchange capacity of about 4.5 to about 5.5 meq/g of dry resin (H+form). The resins differ in physical form. Amberlite IRP-69 consists of irregularly-shaped particles with a size range of about 47 to about 149 μm, produced by milling the parent, large-sized spheres of Amberlite IRP-120. The Dow XYS-40010.00 product consists of spherical particles with a size range of about 45 to about 150 μm.

Another suitable cation exchange resin is Amberlite IRP-64 which is an insoluble, acidic low cross-linked carboxylic acid resin prepared from methacrylic acid and divinyl benzene. IRP-64 consists of particles that range in size from about 25 to about 150 μm, with a mean particle size of about 80 μm. The total cation exchange capacity of IRP-64 is at least about 10 meq/g of resin.

Other cation exchange resins suitable for use in the practice of the present invention include for example Amberlite IRP-88 (Rohm and Haas); Dowex 50V,TX2-400, Dowex 50WX4-400 and Dowex 50WX8400 (Dow Chemical Company; and Purolite C I 15HMR and Purolite C I 02DR (Purolite International Ltd., Hounslow, Great Britain).

In a preferred embodiment of the present invention, the resin is a strongly acidic cation exchange resin. A strongly acidic resin is defined herein as one which has a pKₐ value of less than about 3 measured at 25° C.

In another preferred embodiment of this invention, the resin is an acidic sulfonic acid cation exchange resin. An aspect of this embodiment is an acidic sulfonic acid exchange resin having an exchange capacity below about 8 milliequivalents per gram (meq/g) (e.g., from about 3 to about 6 meq/g) and typically below about 6 meq/g (e.g., from about 3.5 to about 5.5 meq/g). In another aspect of this embodiment, the sulfonic acid exchange resin consists of particles that range in size from about 1 to about 1000 μm and typically range in size from about 2 to about 200 μm. In still another aspect of this embodiment, the resin is Amberlite IRP-69. In yet another aspect of this embodiment, the resin is Amberlite IRP-69 which has been milled to have a mean particle size in a range of from about 1 to about 20 μm (e.g., from about 2 to about 5 μm).

The Compound I/cation exchange resin complex is generally prepared by contacting Compound I (as free base or as a salt) with the resin in an aqueous or organic medium under conditions and for a time sufficient for the compound to complex with the resin, after which the complex is recovered (e.g., separated from the solvent, washed and dried). The medium is suitably water or a polar organic solvent or diluent such as an alcohol (e.g., methanol or ethanol) or an ether (e.g., THF). The pH of aqueous mixtures is typically in a range of from about 1 to about 7. The temperature of the mixture is suitably maintained in a range of from about 15 to about 30° C. (e.g., about 25° C.). In a typical procedure, Compound I is mixed with an aqueous or organic suspension of the cation exchange resin. The adsorption of drug onto the resin can be detected by measuring a change in the pH of an aqueous medium, or by measuring a change in concentration of the counterion (sodium) or of Compound I. The resulting Compound I-resin complex is collected and washed (e.g., with alcohol and/or water) to insure removal of any unbound drug. The resin complex can then be air- or vacuum- dried at room or elevated temperature. Further description of techniques for adsorption of a drug onto the cation exchange resin particles can be found in the art, for example, in U.S. Pat. No. 2,990,332 and U.S. Pat. No. 4,221,778, both of which are herein incorporated by reference in their entireties.

Binding of drug to resin can be accomplished according to four general reactions: (a) resin (Na-form) plus drug (salt form); (b) resin (Na-form) plus drug (as free base); (c) resin (H-form) plus drug (salt form); and (d) resin (H-form) plus drug (as free base). All of these reactions except (d) have cationic by-products, which compete with the cationic form of Compound I for binding sites on the resin and thus can reduce the amount of drug bound at equilibrium. Stoichiometric binding of drug to resin can be accomplished through reaction (d). The binding may be performed, for example, as a batch or column process, as is known in the art.

The complex typically has a weight ratio of Compound I to resin in the range of from about 1:10 to about 10:1 (e.g., from about 1:5 to about 5:1 or from about 1:3 to about 3:1) and more typically from about 1:2 to about 2:1 (e.g., about 1:1).

The amount of the Compound I complexed with the ion exchange resin is suitably in the range from about 15 to about 80% by weight of the Compound I-resin complex, and is typically in the range of from about 20 to about 70% by weight of the Compound I-resin complex. In one aspect of the invention, the amount of Compound I complexed to the ion exchange resin is in the range from about 25 to about 50% by weight of the Compound I-resin complex.

The Compound I-cation exchange resin complex is suitably administered orally. The complex will enter the acidic environment of the stomach (pH of about 2 to 3), wherein the ionized form of Compound I (e.g., ammonium) can be ion exchanged with other cations found in the stomach (e.g., H+). Due to a concentration gradient, the exchanged Compound I cation is released from the polymeric support and will diffuse out of the resin. Compound I can then be absorbed and enter the circulation.

Suitable forms for oral administration include liquid forms such as a suspension and solid forms such as a tablet or capsule. A tablet can be obtained by directly compressing the resin complex of the invention, which can be in the form of granules, with one or more excipients such as diluents, binders, lubricants, glidants, disintegrants, coloring agents and flavoring agents. Excipients suitable for use in preparing the present invention in tablet form include those described in Remington's Pharmaceutical Sciences, 18^{th} edition, 1990, pp. 1635-1638, which is herein incorporated by reference in its entirety. A granule is a solid preparation comprising small particles containing the Compound I-resin complex of the invention and one or more excipients such as lactose, mannitol, microcrystalline cellulose or talc. The granules can be prepared by wet or dry granulation techniques known in the art. A capsule is a container, such as a gelatin shell, for the resin complex of the invention, and can be a dry-filled, wet-filled, liquid-filled or granule-filled shell. A solid form can be of different shapes, sizes or colors depending, for example, on the type and amount of Compound I-resin complex and the excipients (if any) it contains.

Compound I can be administered orally to humans in a dosage range of 0.001 to 1000 mg/kg body weight in divided doses. One preferred dosage range is 0.1 to 200 mg/kg body weight orally in divided doses. Another preferred dosage range is 0.5 to 100 mg/kg body weight orally in divided doses. For oral administration, the compositions of the present invention are preferably provided in the form of capsules or tablets containing 1 to 1000 milligrams of the active ingredient, particularly 1, 5, 10, 15. 20, 25, 50, 75, 100, 150, 200, 250, 300, 400, 500, 600, 750, 800, 900, and 1000 milligrams of the active ingredient for the symptomatic adjustment of the dosage to the patient to be treated. It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Oral administration to human subjects suitably follows a meal or a soda, which typically will increase stomach acidity and thus promote release of the drug from the resin.

In a preferred embodiment, the oral dosage form is a capsule containing from about 10 to about 1000 mg of Compound I complexed with a cation exchange resin. In one aspect of this embodiment, the cation exchange resin is Amberlite IRP-69, wherein the amount of Compound I complexed with the resin is in the range from about 15 to about 75% by weight of the Compound I-resin complex.

The present invention also includes combinations of the composition of the invention comprising the Compound I-cation exchange resin complex with one or more agents useful in the treatment of HIV infection and/or AIDS. For example, the compounds of this invention may be effectively administered in combination with effective amounts of the HIV/AIDS antivirals, imunomodulators, antiinfectives, or vaccines, such as those in Table 1 as follows:

TABLE 1
HIV/AIDS ANTIVIRALS, IMUNOMODULATORS,
ANTIINFECTIVES, AND OTHER TREATMENTS

| | | |
|---|---|---|
| Drug Name | Manufacturer | Indication |

ANTIVIRALS

| | | |
|---|---|---|
| Amprenavir | Glaxo Wellcome | HIV infection, AIDS, |
| 141 W94 | | ARC |
| GW 141 | | (protease inhibitor) |
| Abacavir | Glaxo Welcome | HIV infection, AIDS, |
| GW 1592 | | ARC |
| 1592U89 | | (reverse transcriptase |
| | | inhibitor) |
| Acemannan | Carrington Labs | ARC |
| | (Irving, TX) | |
| Acyclovir | Burroughs Wellcome | HIV infection, AIDS, ARC, |
| | | in combination with AZT |
| AD-439 | Tanox Biosystems | HIV infection, AIDS, ARC |
| AD-519 | Tanox Biosystems | HIV infection, AIDS, ARC |
| Adefovir dipivoxil | Gilead Sciences | HIV infection |
| AL-721 | Ethigen | ARC, PGL, HIV positive, |
| | (Los Angeles, CA) | AIDS |
| Alpha Interferon | Glaxo Wellcome | Kaposi's sarcoma, HIV, in |
| | | combination w/Retrovir |
| Ansamycin | Adria Laboratories | ARC |
| LM 427 | (Dublin, OH) Erbamont | |
| | (Stamford, CT) | |
| Antibody which | Advanced Biotherapy | AIDS, ARC |
| neutralizes pH | Concepts | |
| labile alpha aberrant | (Rockville, MD) | |
| Interferon | | |
| AR177 | Aronex Pharm | HIV infection, AIDS, ARC |
| beta-fluoro-ddA | Nat'l Cancer Institute | AIDS-associated diseases |
| BMS-232623 | Bristol-Myers Squibb/ | HIV infection, AIDS, |
| (CGP-73547) | Novartis | ARC |
| | | (protease inhibitor) |
| BMS-234475 | Bristol-Myers Squibb/ | HIV infection, AIDS, |
| (CGP-61755) | Novartis | ARC |
| | | (protease inhibitor) |
| CI-1012 | Warner-Lambert | HIV-1 infection |
| Cidofovir | Gilead Science | CMV retinitis, herpes, |
| | | papillomavirus |
| Curdlan sulfate | AJI Pharma USA | HIV infection |
| Cytomegalovirus immune | MedImmune | CMV retinitis |
| globin | | |
| Cytovene | Syntex | sight threatening CMV |
| Ganciclovir | | peripheral CMV |
| | | retinitis |
| Delaviridine | Pharmacia-Upjohn | HIV infection, AIDS, |
| | | ARC |
| | | (protease inhibitor) |
| Dextran Sulfate | Ueno Fine Chem. | AIDS, ARC, HIV |
| | Ind. Ltd. (Osaka, Japan) | positive asymptomatic |
| ddC | Hoffman-La Roche | HIV infection, AIDS, ARC |
| Dideoxycytidine | | |
| ddI | Bristol-Myers Squibb | HIV infection, AIDS, ARC; |
| Dideoxyinosine | | combination with AZT/d4T |
| DMP-450 | AVID | HIV infection, AIDS, |
| | (Camden, NJ) | ARC |
| | | (protease inhibitor) |
| EL10 | Elan Corp, PLC | HIV infection |
| | (Gainesville, GA) | |
| Efavirenz | DuPont (SUSTIVA ®), | HIV infection, AIDS, |
| (DMP 266) | Merck (STOCRIN ®) | ARC |
| (-) 6-Chloro-4(S)- | | (non-nucleoside RT |
| cyclopropylethynyl- | | inhibitor) |
| 4(S)-trifluoro-methyl- | | |
| 1,4-dihydro-2H-3,1- | | |
| benzoxazin-2-one, | | |
| Famciclovir | Smith Kline | herpes zoster, herpes |
| | | simplex |
| FTC | Emory University | HIV infection, AIDS, ARC |
| | | (reverse transcriptase |
| | | inhibitor) |
| GS 840 | Gilead | HIV infection, AIDS, ARC |
| | | (reverse transcriptase |
| | | inhibitor) |
| HBY097 | Hoechst Marion Roussel | HIV infection, AIDS, ARC |
| | | (non-nucleoside reverse |
| | | transcriptase inhibitor) |
| Hypericin | VIMRx Pharm. | HIV infection, AIDS, ARC |
| Recombinant Human | Triton Biosciences | AIDS, Kaposi's sarcoma, |
| Interferon Beta | (Almeda, CA) | ARC |
| Interferon alfa-n3 | Interferon Sciences | ARC, AIDS |
| Indinavir | Merck | HIV infection, AIDS, ARC, |
| | | asymptomatic HIV positive, |
| | | also in combination with |
| | | AZT/ddI/ddC |
| ISIS 2922 | ISIS Pharmaceuticals | CMV retinitis |
| KNI-272 | Nat'l Cancer Institute | HIV-assoc. diseases |
| Lamivudine, 3TC | Glaxo Wellcome | HIV infection, AIDS, |
| | | ARC (reverse |
| | | transcriptase inhibitor); |
| | | also with AZT |
| Lobucavir | Bristol-Myers Squibb | CMV infection |
| Nelfinavir | Agouron | HIV infection, AIDS, |
| | Pharmaceuticals | ARC (protease inhibitor) |
| Nevirapine | Boeheringer | HIV infection, AIDS, |
| | Ingleheim | ARC (protease inhibitor) |
| Novapren | Novaferon Labs, Inc. | HIV inhibitor |
| | (Akron, OH) | |
| Peptide T | Peninsula Labs | AIDS |
| Octapeptide | (Belmont, CA) | |
| Sequence | | |
| Trisodium | Astra Pharm. | CMV retinitis, HIV infection, |
| Phosphonoformate | Products, Inc | other CMV |
| | | infections |
| PNU-140690 | Pharmacia Upjohn | HIV infection, AIDS, ARC |
| | | (protease inhibitor) |
| Probucol | Vyrex | HIV infection, AIDS |
| RBC-CD4 | Sheffield Med. Tech | HIV infection, AIDS, |
| | (Houston TX) | ARC |
| Ritonavir | Abbott | HIV infection, AIDS, |
| (ABT-538) | | ARC (protease inhibitor) |
| Saquinavir | Hoffmann-LaRoche | HIV infection, AIDS, |
| | | ARC (protease inhibitor) |
| Stavudine; d4T | Bristol-Myers Squibb | HIV infection, AIDS, ARC |
| Didehydrodeoxy- | | |
| thymidine | | |
| Valaciclovir | Glaxo Wellcome | genital HSV & CMV |
| | | infections |
| Virazole | Viratek/ICN | asymptomatic HIV |
| Ribavirin | (Costa Mesa, CA) | positive, LAS, ARC |
| VX-478 | Vertex | HIV infection, AIDS, ARC |
| Zalcitabine | Hoffmann-La Roche | HIV infection, AIDS, ARC, |
| | | with AZT |
| Zidovudine; AZT | Glaxo Wellcome | HIV infection, AIDS, ARC, |
| | | Kaposi's sarcoma in |
| | | combination with other |
| | | therapies (reverse |
| | | transcriptase inhibitor) |
| ABT-378; Lopinavir | Abbott | HIV infection, AIDS, ARC |
| | | (protease inhibitor) |
| ABT-378/r; contains | Abbott | HIV infection, AIDS, ARC |
| lopinavir and ritonavir; | | (protease inhibitor) |
| Kaletra | | |
| JE2147/AG1776 | Agouron | HIV infection, AIDS, ARC |
| | | (protease inhibitor) |
| T-20 | Trimeris | HIV infection, AIDS, ARC |
| | | (fusion inhibitor) |
| T-1249 | Trimeris | HIV infection, AIDS, ARC |
| | | (fusion inhibitor) |
| BMS 232632; | Bristol-Myers-Squibb | HIV infection, AIDS, ARC |
| Atazanavir; Zrivada | | (protease inhibitor) |
| PRO 542 | Progenics | HIV infection, AIDS, ARC |
| | | (attachment inhibitor) |
| PRO 140 | Progenics | HIV infection, AIDS, ARC |
| | | (CCR5 co-receptor inhibitor) |
| TAK-779 | Takeda | HIV infection, AIDS, ARC |
| | | (injectable CCR5 receptor |
| | | antagonist) |
| DPC 681 & DPC 684 | DuPont | HIV infection, AIDS, ARC |
| | | (protease inhibitors) |
| DPC 961 & DPC 083 | DuPont | HIV infection, AIDS, ARC |
| | | (nonnucleoside reverse |
| | | transcriptase inhibitors) |
| Trizivir (contains abacavir, | GlaxoSmithKline | HIV infection, AIDS, ARC |
| lamiduvidne, and | | (reverse transcriptase |
| zidovudine) | | inhibitors) |
| tipranavir | Boehringer Ingelheim | HIV infection, AIDS, ARC |
| | | (protease inhibitor) |
| tenofovir; Viread | Gilead | HIV infection, AIDS, ARC |
| | | (nucleotide reverse |
| | | transcriptase inhibitor) |
| TMC-120 & TMC-125 | Tibotec | HIV infection, AIDS, ARC |
| | | (non-nucleotide reverse |
| | | transcriptase inhibitor) |
| TMC-126 | Tibotec | HIV infection, AIDS, ARC |
| | | (protease inhibitor) |

IMMUNO-MODULATORS

| | | |
|---|---|---|
| AS-101 | Wyeth-Ayerst | AIDS |
| Bropirimine | Pharmacia Upjohn | advanced AIDS |
| Acemannan | Carrington Labs, Inc. | AIDS, ARC |
| | (Irving, TX) | |
| CL246, 738 | American Cyanamid | AIDS, Kaposi's sarcoma |
| | Lederle Labs | |
| EL10 | Elan Corp, PLC | HIV infection |
| | (Gainesville, GA) | |
| FP-21399 | Fuki ImmunoPharm | blocks HIV fusion with |
| | | CD4+ cells |
| Gamma Interferon | Genentech | ARC, in combination w/TNF |
| | | (tumor necrosis factor) |
| Granulocyte | Genetics Institute | AIDS |
| Macrophage Colony | Sandoz | |
| Stimulating | | |
| Factor | | |
| Granulocyte | Hoeschst-Roussel | AIDS |
| Macrophage Colony | Immunex | |
| Stimulating | | |
| Factor | | |
| Granulocyte | Schering-Plough | AIDS, combination w/AZT |
| Macrophage Colony | | |
| Stimulating Factor | | |
| HIV Core Particle | Rorer | seropositive HIV |
| Immunostimulant | | |
| IL-2 | Cetus | AIDS, in combination |
| Interleukin-2 | | w/AZT |
| IL-2 | Hoffman-La Roche | AIDS, ARC, HIV, in |
| Interleukin-2 | Immunex | combination w/AZT |
| IL-2 | Chiron | AIDS, increase in CD4 cell |
| Interleukin-2 | | counts |
| (aldeslukin) | | |
| Immune Globulin | Cutter Biological | pediatric AIDS, in |
| Intravenous | (Berkeley, CA) | combination w/AZT |
| (human) | | |
| IMREG-1 | Imreg | AIDS, Kaposi's |
| | (New Orleans, LA) | sarcoma, ARC, PGL |
| IMREG-2 | Imreg | AIDS, Kaposi's sarcoma, |
| | (New Orleans, LA) | ARC, PGL |
| Imuthiol Diethyl | Merieux Institute | AIDS, ARC |
| Dithio Carbamate | | |
| Alpha-2 | Schering Plough | Kaposi's sarcoma w/AZT, |
| Interferon | | AIDS |
| Methionine- | TNI Pharmaceutical | AIDS, ARC |
| Enkephalin | (Chicago, IL) | |
| MTP-PE | Ciba-Geigy Corp. | Kaposi's sarcoma |
| Muramyl-Tripeptide | | |
| Granulocyte | Amgen | AIDS, in combination |
| Colony Stimulating | | w/AZT |
| Factor | | |
| Remune | Immune Response Corp. | immunotherapeutic |
| rCD4 | Genentech | AIDS, ARC |
| Recombinant | | |
| Soluble Human CD4 | | |
| rCD4-IgG | | AIDS, ARC |
| hybrids | | |
| Recombinant | Biogen | AIDS, ARC |
| Soluble Human CD4 | | |
| Interferon | Hoffman-La Roche | Kaposi's sarcoma, AIDS, |
| Alfa 2a | | ARC, in combination w/AZT |
| SK&F106528 | Smith Kline | HIV infection |
| Soluble T4 | | |
| Thymopentin | Immunobiology | HIV infection |
| | Research Institute | |
| Tumor Necrosis | Genentech | ARC, in combination |
| Factor; TNF | | w/gamma Interferon |
| etanercept | Immunex Corp | rheumatoid arthritis |
| | (Enbrel ®) | |
| infliximab | Centocor (Remicade ®) | rheumatoid arthritis and |
| | | Crohn's disease |

ANTI-INFECTIVES

| | | |
|---|---|---|
| Clindamycin with | Pharmacia Upjohn | PCP |
| Primaquine | | |
| Fluconazole | Pfizer | cryptococcal meningitis, |
| | | candidiasis |
| Pastille | Squibb Corp. | prevention of oral candidiasis |
| Nystatin Pastille | | |
| Ornidyl | Merrell Dow | PCP |
| Eflornithine | | |
| Pentamidine | LyphoMed | PCP treatment |
| Isethionate (IM & IV) | (Rosemont, IL) | |
| Trimethoprim | | antibacterial |
| Trimethoprim/sulfa | | antibacterial |
| Piritrexim | Burroughs Wellcome | PCP treatment |
| Pentamidine | Fisons Corporation | PCP prophylaxis |
| isethionate for | | |
| inhalation | | |
| Spiramycin | Rhone-Poulenc | cryptosporidia diarrhea |
| Intraconazole- | Janssen Pharm. | histoplasmosis; cryptococcal |
| R51211 | | meningitis |
| Trimetrexate | Warner-Lambert | PCP |

OTHER

| | | |
|---|---|---|
| Daunorubicin | NeXstar, Sequus | Karposi's sarcoma |
| Recombinant Human | Ortho Pharm. Corp. | severe anemia assoc. with |
| Erythropoietin | | AZT therapy |
| Recombinant Human | Serono | AIDS-related wasting, |
| Growth Hormone | | cachexia |
| Leukotriene B4 Receptor | — | HIV infection |
| Antagonist | | |
| Megestrol Acetate | Bristol-Myers Squibb | treatment of anorexia assoc. w/AIDS |
| Soluble CD4 Protein and | — | HIV infection |
| Derivatives | | |
| Testosterone | Alza, Smith Kline | AIDS-related wasting |
| Total Enteral | Norwich Eaton | diarrhea and malabsorption, |
| Nutrition | Pharmaceuticals | related to AIDS |

It will be understood that the scope of combinations of the compounds of this invention with HIV/AIDS antivirals, immunomodulators, anti-infectives or vaccines is not limited to the list in Table 1 above, but includes in principle any combination with any pharmaceutical composition useful for the treatment of HIV infection and/or AIDS.

One suitable combination is the composition of the present invention and a nucleoside inhibitor of HIV reverse transcriptase such as AZT, 3TC, ddC, or ddI. Another suitable combination is a composition of the present invention and a non-nucleoside inhibitor of HIV reverse transcriptase, such as efavirenz, and optionally a nucleoside inhibitor of HIV reverse transcriptase, such as AZT, 3TC, ddC or ddI.

Still another suitable combination is any one of the combinations in the preceding paragraph, further comprising an additional HIV protease inhibitor such as indinavir, nelfinavir, ritonavir, saquinavir, amprenavir, or abacavir. An aspect of this combination is the combination wherein the additional inhibitor of HIV protease is the sulfate salt of indinavir. Another aspect of this combination is the combination in which the additional protease inhibitor is selected from nelfinavir and ritonavir. Still another aspect of this combination is the combination in which the additional inhibitor of HIV protease is saquinavir, which is typically administered in a dosage of 600 or 1200 mg tid.

Other suitable combinations include a composition of the present invention with the following: (1) efavirenz, optionally with AZT and/or 3TC and/or ddI and/or ddC, and optionally with indinavir; (2) any of AZT and/or ddI and/or ddC and/or 3TC, and optionally with indinavir; (3) d4T and 3TC and/or AZT; (4) AZT and 3TC; and (5) AZT and d4T.

Another aspect of the present invention is co-administration of the composition of the present invention with an inhibitor of cytochrome P450 monooxygenase in an amount effective to improve the pharmacokinetics of the compound. Compounds of Formula (I) can be metabolized, at least in part, by cytochrome P450 (CYP3A4). Accordingly, co-administration of the composition of the invention with a cytcochrome P450 inhibitor can improve the pharmacokinetic profile of compounds of Formula (I) in subjects (e.g., humans); i.e., co-administration can increase Cₘₐₓ (the maximum plasma concentration of Compound I), AUC (area under the curve of plasma concentration of the compound versus time), and/or the half-life of the compound. Suitable P450 inhibitors include, but are not limited to, indinavir and ritonavir. It is to be understood that the primary role of indinavir and ritonavir in this circumstance is as a pharmacokinetic modulator and not as a protease inhibitor; i.e., an amount of indinavir or ritonavir which is effective for improving the pharmacokinetics of the compound can provide a secondary or even negligible contribution to the antiviral effect.

The composition of the present invention can also be administered in combination with an HIV integrase inhibitor such as a compound described in WO 99/62520, WO 99/62513, or WO 99/62897. The composition of the present invention can also be administered in combination with a CCR5 receptor antagonist, such as a compound described in WO 00/59502 or WO 00/59503.

In the above-described combinations, the composition of the present invention and other active agents may be administered together or separately. In addition, the administration of one agent may be prior to, concurrent with, or subsequent to the administration of other agent(s). These combinations may have unexpected or synergistic effects on limiting the spread and degree of infection of HIV.

Efavirenz is (−)-6-chloro-4-cyclopropylethynyl-4-trifluoromethyl-1,4-dihydro-2H-3,1-benzoxazin-2-one, also known as DMP-266 or SUSTIVA® (DuPont) or STOCRIN® (Merck). Efavirenz and its utility as an HIV reverse transcriptase inhibitor is described in U.S. Pat No. 5,519,021 and in the corresponding PCT published application, WO 95/20389. Efavirenz can be synthesized by the protocol of U.S. Pat. No. 5,633,405. Additionally, the asymmetric synthesis of an enantiomeric benzoxazinone by a highly enantioselective acetylide addition and cyclization sequence is described in Thompson et al., Tetrahedron Letters 1995, 36: 8937-40, as well as in the PCT publication, WO 96/37457.

AZT is 3′-azido-3′-deoxythymidine, is also known as zidovudine, and is available from Burroughs-Wellcome under the tradename RETROVIR®. Stavudine is 2′,3′-didehydro-3′-deoxythymidine, is also known as 2′,3′-dihydro-3′-deoxythymidine and d4T, and is available from Bristol-Myers Squibb under the tradename ZERIT®. 3TC is (2R-cis)-4-amino-1-[2-(hydroxymethyl)-1,3-oxathiolan-5-yl]-2(1H)-pyrimidinone, is also known as (−)-1-[(2R,5S)-2-(hydroxymethyl)-1,3-oxathiolan-5-yl]cytosine and lamivudine, and is available from Glaxo Wellcome under the tradename EPIVIR®. ddC is 2′,3′-dideoxycytidine, is also known as zalcitabine, and is available from Hoffman LaRoche under the tradename HIVID®. ddI is 2′,3′-dideoxyinosine, is also known as didanosine, and is available from Bristol-Myers-Squibb under the tradename VIDEX®. The preparation of ddC, ddI and AZT are also described in EPO 0,484,071.

Indinavir is N-(2(R)-hydroxy-1(S)-indanyl)-2(R)-phenylmethyl-4-(S)-hydroxy-5-(1-(4-(3-pyridyl-methyl)-2(S)-N′-(t-butylcarboxamido)-piperazinyl))-pentaneamide, and can be prepared as described in U.S. Pat. No. 5,413,999. Indinavir is generally administered as the sulfate salt at a dosage of 800 mg three times a day. Indinavir sulfate is available from Merck under the tradename CRIXIVAN®.

Ritonavir is [5S-(5R*,8R*,10R*, 11R*)]-10-hydroxy-2-methyl-5-(1-methylethyl)-1-[2-(1-methylethyl)-4-thiazolyl]-3,6-dioxo-8,11-bis(phenylmethyl)-2,4,7,12-tetraazatridecan-13-oic acid 5-thiazolylmethyl ester, also known as 5-thiazolylmethyl [(aS)-α-[(1S,3S)-1-hydroxy-3-[(2S)-2-[3-[(2-isopropyl-4-thiazolyl)methyl]-3-methylureido]-3-methylbutyramido]-4-phenylbutyl]phenethyl]carbamate. It is available from Abbott under the tradename NORVIR®. Ritonavir can be prepared as described in U.S. Pat. No. 5,484,801.

Nelfinavir is [3S-[2(2S *,3S*),3a,4ab,8ab]]-N-(1,1-dimethylethyl)decahydro-2-hydroxy-3-[(3-hydroxy-2-methylbenzoyl)amino]-4-(phenylthio)butyl]-3-isoquinolinecarboxamide, also known as (3S,4aS,8aS)-N-tert-Butyl-2-[(2R,3R)-3-(3,2-crestoamido)-2-hydroxy-4-(phenylthio)butyl]decahydro-3-isoquinolinecarboxamide. VIRACEPT®, the monomethanesulfonate salt of nelfinavir (nelfinavir mesylate) is commerically available from Agouron. Nelfinavir can be prepared as described in U.S. Pat. No. 5,484,926.

Saquinavir is N-tert-butyl-decahydro-2-[2(R)-hydroxy-4-phenyl-3(S)-[[N-(2-quinolylcarbonyl)-L-asparaginyl]amino]butyl]-(4aS,8aS)-isoquinoline-3(S)-carboxamide. Saquinavir can be prepared in accordance with procedures disclosed in U.S. Pat. No. 5,196,438. INVIRASE® (saquinavir mesylate) is available from Roche Laboratories.

Amprenavir is 4-amino-N-((2 syn,3S)-2-hydroxy-4-phenyl-3-((S)-tetrahydrofuran-3-yloxycarbonylamino)-butyl)-N-isobutyl-benzenesulfonamide, also known as Compound 168 and 141 W94. Amprenavir is an aspartyl protease inhibitor that can be prepared by following the procedures described in U.S. Pat. No. 5,585,397. Amprenavir is available under the tradename AGENERASE® from Glaxo Wellcome. Amprenavir can be prepared as described in U.S. Pat. No. 5,783,701.

Abacavir is (1S ,4R)-cis-4-[2-amino-6-(cyclopropylamino)-9H-purin-9-yl]-2-cyclopentene-1-methanol, also known as 1592U89. Abacavir can be prepared by following the protocol of EP 0434450.

Abbreviations used in the instant specification, particularly the Schemes and Examples, are as follows:

ACN=acetonitrile

AcOH=acetic acid

BOC or Boc=t-butyloxycarbonyl

BOC-ON=2-(tert-butoxycarbonylamino)-2-phenyl acetonitrile

Bu=butyl

DMF=dimethylformamide

EDC=1-ethyl-3-(3-dimethylaminopropyl)carbodiimide

Et=ethyl

Et₂O=diethyl ether

EtOAc=ethyl acetate

EtOH=ethanol

HOAT=1-hydroxy-7-azabensotriazole

HOBT=1-hydroxy benzotriazole hydrate

HPLC=high performance liquid chromatography

KF=Karl Fisher titration for water

LC=liquid chromatography

NMR=nuclear magnetic resonance

Pd(dppf)Cl₂=1,1′-bis(diphenylphosphino)ferrocene palladium dichloride

TBDC=di t-butyl dicarbonate

THF=tetrahydrofuran

TLC=thin layer chromatgraphy

TMEDA=N,N,N′,N′-tetramethylethylenediamine

The compounds of Formula (I) complexed with a cation exchange resin in the present invention can be readily prepared according to the following reaction schemes and examples, or modifications thereof, using readily available starting materials, reagents and conventional synthesis procedures. In these reactions, it is also possible to make use of variants which are themselves known to those of ordinary skill in this art, but are not mentioned in greater detail. Furthermore, other methods for preparing compounds of the invention will be readily apparent to the person of ordinary skill in the art in light of the following reaction schemes and examples. Unless otherwise indicated, all variables are as defined above.

The preparation of the compounds employed in the complexes of the present invention can be carried out in sequential or convergent synthetic routes, as shown in Schemes 1-8 below. A compound of Formula (I) can be prepared in accordance with Scheme 1, wherein Compound I is readily prepared via literature procedures described in Dorsey et al., J. Med. Chem. 1994, 37: 3443-3451, and also in U.S. Pat. No. 5,413,999. Treatment of the hydroxyl compound 1 with triflic anhydride and lutidine in an inert solvent such as dichloromethane provides triflate 2. Displacement of the triflate with piperazine 3 occurs on heating in an inert solvent such as isopropanol to give lactone 4. Hydrolysis of lactone 4 with an aqueous lithium hydroxide provides the hydroxy acid which is conveniently protected with a standard silyl protecting group such as t-butyldimethylsilyl by reaction with either t-butyldimethylsilyl chloride in the presence of imidazole in an inert solvent or the reaction with the silyl triflate and diisopropyl ethylamine in an inert solvent such as dichloromethane. Mild aqueous hydrolysis of the silyl ester provides the protected hydroxy-acid 5. Amide coupling of compound 5 with NH₂R⁵ to obtain 6 is typically performed by the carbodiimide method with reagents such as 1-ethyl-3-(3-dimethylaminopropyl) carbodiimide (EDC) and HOBT in an inert solvent such as dichloromethane. Other methods of forming the amide or peptide bond include, but are not limited to, the synthetic routes via an acid chloride, azide, mixed anhydride or activated ester. The silyl protecting group is removed with fluoride to arrive at compound 7. The BOC protecting group on the amine is then removed with a strong acid such as trifluoroacetic acid or hydrochloric acid in an alcoholic solvent such as methanol to give the penultimate intermediate 8. Penultimate 8 is then reacted with the desired aldehyde 9 and a reducing agent such as sodium cyanoborohydride or sodium triacetoxyborohydride in an inert solvent such as dichloromethane to give compound 10.

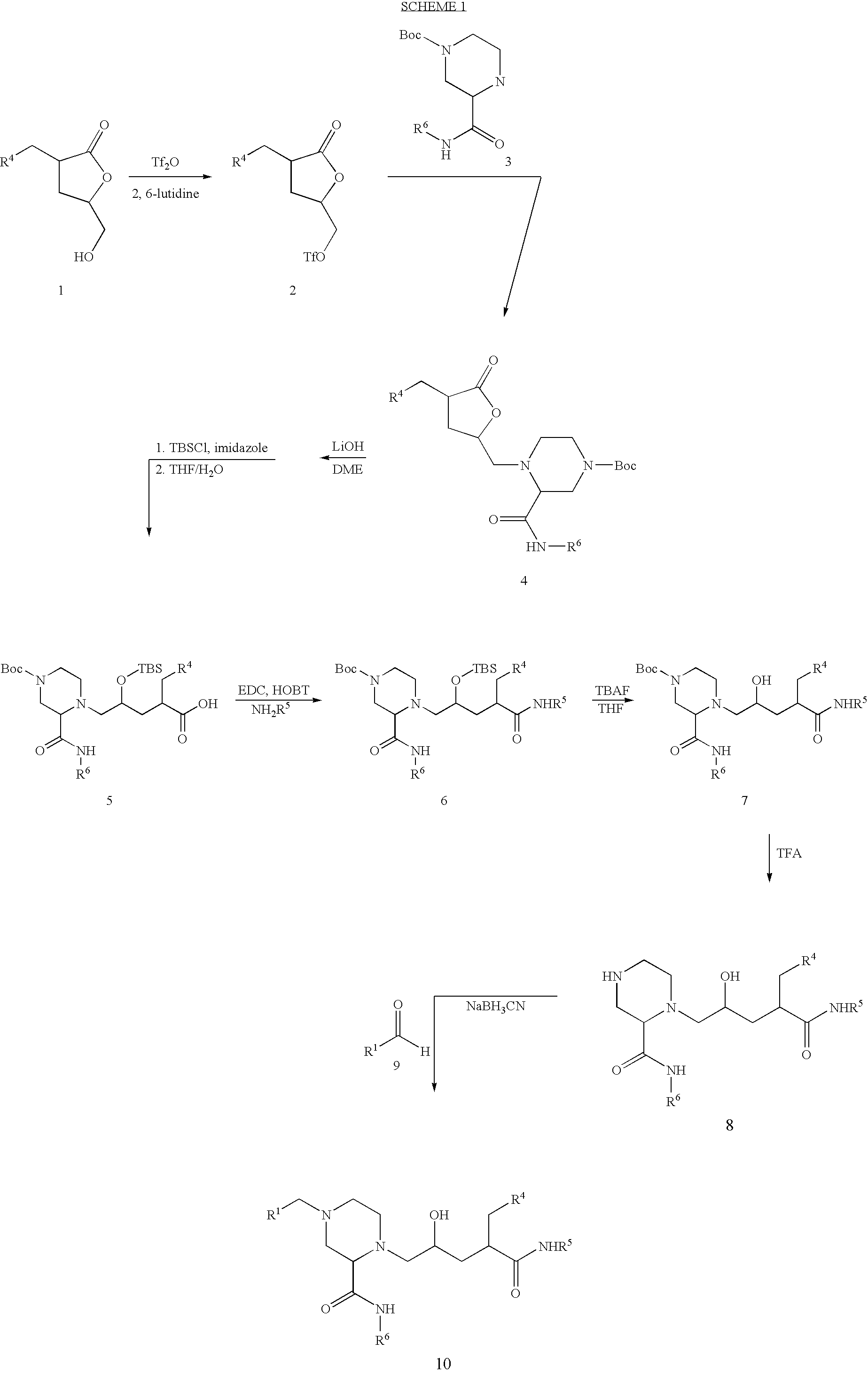

A more convergent route to compounds employed in the present invention is presented in Scheme 2, below. The orthogonally protected piperazine 11 can be selectively deprotected. The BOC protecting group can be removed by treatment with strong acids such as trifluoroacetic acid in dichloromethane or HCl in methanol. The resulting amine 12 can then be reacted with an aldehyde in the presence of a reducing agent such as sodium cyanoborohydride or sodium triacetoxyborohydride to give piperazine 13. Removal of the Alloc protecting group is readily accomplished with a palladium catalyst in the presence of a nucleophilic trapping agent such as 1,3-dimethylbarbituric acid or as in J. Org. Chem. 1993, 58, 6109-6113. Displacement of the triflate of 2 with piperazine 14, as in Scheme 1 gives lactone 15 which is then converted into compounds employed in the present invention following the route depicted in Scheme 1.

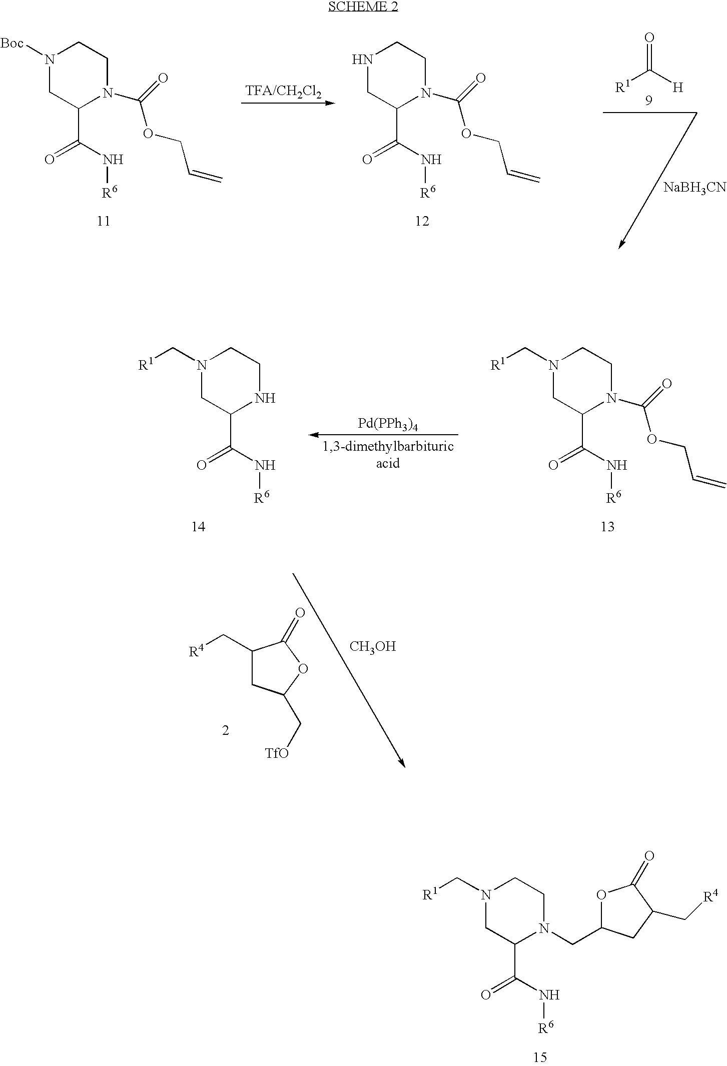

An alternative route to the instant compounds is presented in Scheme 3, as exemplified for NH₂R⁵=aminoindanol. Compound 16 can be easily prepared according to the procedures described in the literature including, but not limited to, those described in Tetrahedron Letters 1995, 36: 2195-2198 and U.S. Pat. No. 5,646,148. As shown in Part A of Scheme 3, the epoxide opening can be carried out by heating piperazine 3 and the epoxide in an inert solvent. Acidic removal of the protecting groups can be accomplished by treatment with hydrochloric acid in an alcoholic solvent such as methanol, ethanol or isopropanol. The resulting intermediate 18 is then reductively aminated as in Scheme 1 to provide the compounds employed in the present invention. Alternatively, as shown in Part B of Scheme 3, the epoxide opening can be preformed with fully elaborated piperazine 14 to give 20. Once again the protecting group is removed with strong acid to give 19.

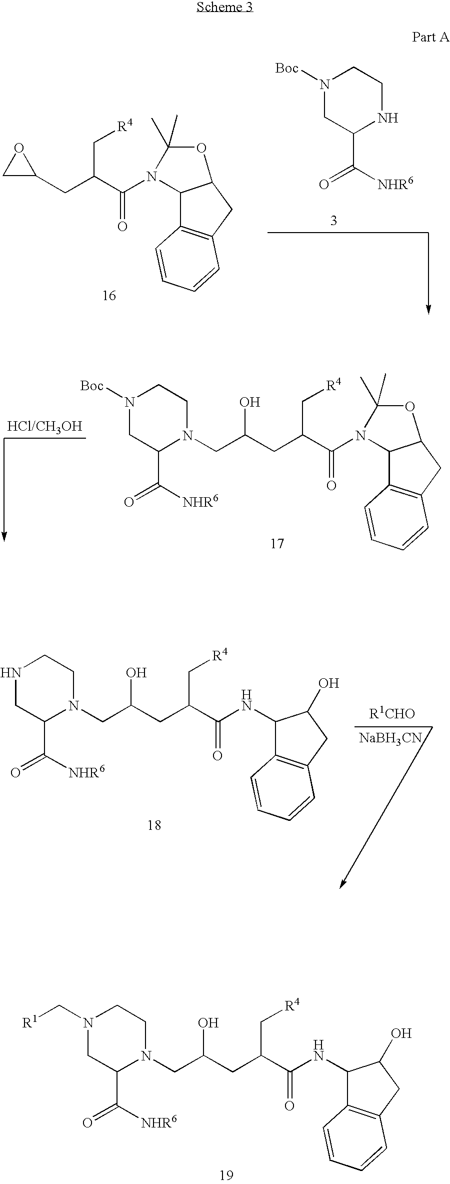

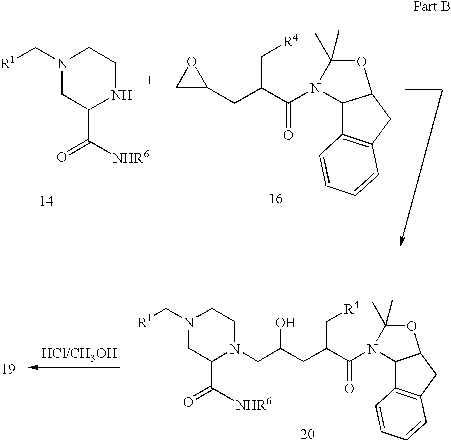

Intermediates of formula NH₂R⁵ can be readily prepared via the literature procedures including, but not limited to, those found in Tetrahedron Letters 1991, 32: 711-714, Tetrahedron Letters 1995, 36: 3993-3996 and Synthesis 1998, 938-961. A procedure for preparing cis-aminochromanols by the stereoselective hydroge bromide-promoted hydrogenation of an α-hydroxyoxime is described in Davies et al., Tetrahedron Letters 2000, 41: 8021-8025.

Piperazine intermediates are readily prepared from the known piperazine carboxylic acid 21, which can be prepared as described in Hel. Chem. Acta. 1960, 43: 888-896. Selective monoprotection of the piperazine is carried out using BOC anhydride as described in Tetrahedron Letters 1989, 30: 5193-5196. The remaining unprotected amine can then be protected with any number of chloroformates including allyl chloroformate or benzyl chloroformate to give 23. Amide couplings of 23 with NH₂R⁶ to give 24 are performed using standard amide coupling reactions as described above. Many NH₂R⁶ amines are commercially available and others can be prepared via literature methods including, but not limited to, those described in Tetrahedron Letters 1999, 40, 3831-3834. Acidic removal of the BOC protecting group as before gives 25. The Alloc group can be removed as before. The CBZ group is readily removed by hydrogenolysis with a palladium catalyst under a hydrogen atmosphere in an alcoholic solvent such as methanol or ethanol. Removal of the protecting groups can also be accomplished by a number of methods known in the art, such as those described in Greene, Protective Groups in Organic Synthesis, John Wiley and Sons, New York, 1991. These deprotected intermediates are then carried onto compounds of the instant invention via the synthetic routes shown in Schemes 1, 2 and 3.

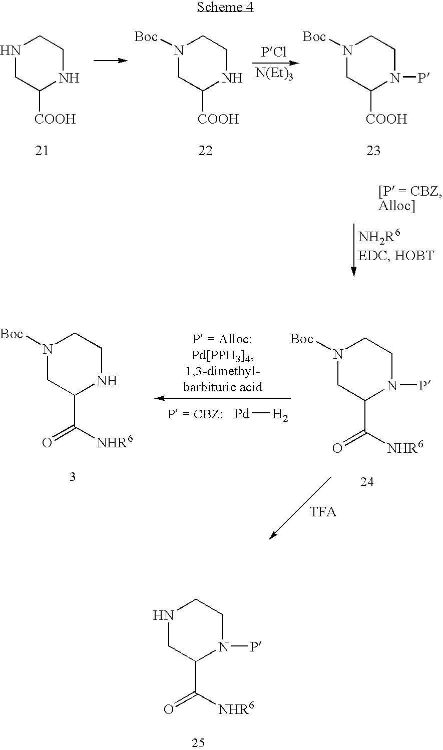

The desired aldehyde intermediates are, in many cases, commercially available (e.g., Aldrich Chemical). Other aldehydes of interest can be prepared by literature methods including classical methods familiar to those skilled in the art. Stille and Suzuki coupling of commercially and readily available aryl and heteroaryl halides, aryl trialkylstannanes, and arylboronic acids also provides the desired aldehydes as exemplified for bromofuran in Scheme 5 below. Aldehyde 27 can be reacted with trialkylarylstannane 26 in the presence of a palladium catalyst by the method of Gronowitz et al., J. Heterocyclic Chem. 1995, 35: 771, to give 28. Alternatively, trialkylstannane 30 can be coupled with arylhalides such as 29 to give 31 which can be deprotected under mild conditions with dilute hydrochloric acid to give aldehyde 28. Other aldehydes are available via metal halogen exchange followed by anion quenching with DMF as described by Vogel et al., J. Chem. Soc. Perkin Trans 1, 1974, 37. Metalation of a biaryl or heterobiaryl compound such as 32 with a strong base such as n-butyllithium at low temperature in an inert solvent such as THF followed by anion trapping with DMF also provides aldehydes such as 28.

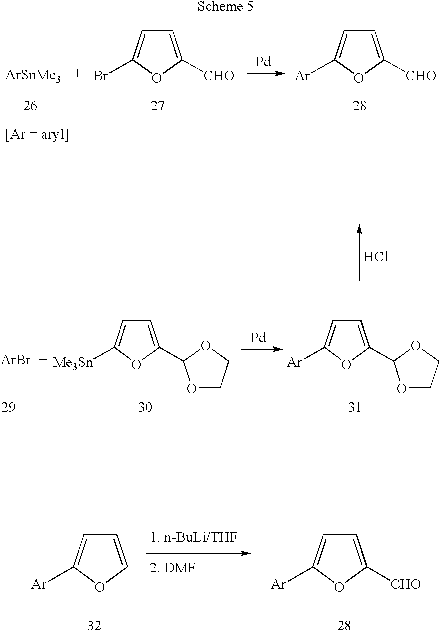

When R² and R³ are alkyl, the necessary intermediates can be formed as shown in Scheme 6 below. Piperazine 12 can be treated with TMSCN and a ketone in acetic acid to give intermediate 34 according to the method described in J. Org. Chem. 1990, 55, 4207-4209. The Alloc protecting group is removed as described in Scheme 4 and the resulting intermediate, 35, is then treated with an excess of a Grignard to give the gem-dialkyl compound 14A. This intermediate is then converted to the compounds employed in the present invention via chemistry described in Schemes 2 and 3 above.

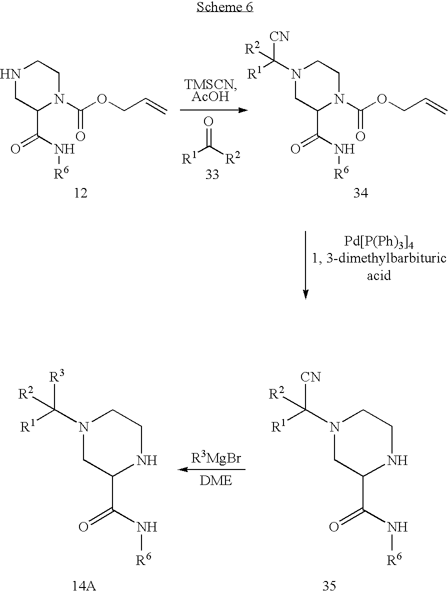

An additional route to intermediates such as 14A, where R² and R³ are alkyl or cycloalkyl, is depicted in Scheme 7, below. Alkylation of piperazine 25, where P′ is an appropriate protecting group such as those described above, with alkylating agent 36, is conveniently carried out in the presence of copper oxide, copper, and a tertiary amine base according to methods described in J. Org. Chem 1996, 61: 6517-6522, J. Am. Chem. Soc. 1960, 4908, and J. Org. Chem. 1994, 59: 2282-2284, where R² and R³ are alkyl or cycloalkyl and X is a leaving group such as bromine, chlorine, mesylate, triflate, or phosphonate. Heterocycles of interest can be prepared from the acetylenic piperazine 37 using chemistry known to those skilled in the art. For example, intermediates such as 39 can be formed by the reaction of iodo or bromo phenols such as 38 with 37 according to the procedures of Castro et al., J. Org. Chem. 1966, 31: 4071-4078, Larock et al., J. Org. Chem. 1995, 60: 3270, or Arcadi et al., Synthesis 1986, 749. Triazole intermediates 41 are readily available from the reaction of 37 and aryl or heteroaryl azides as shown for phenylazide 40 in an inert high boiling solvent such as dichlorobenzene according to the method of Sakamoto et. al. as described in Heterocycles 1993, 35: 1273. Sydnones, such as 42, are available by procedures detailed in J. Heterocycl. Chem. 1992, 29: 1013-1015. They can be reacted with 37 to give pyrazoles such as 43 according to the procedure of Gotthardt et al. as described in Chem. Ber. 1968, 101: 536. Isoxazole intermediates such as 45 can be formed by treatment of the piperazine 37 with nitrones like 44 in a high boiling solvent such as nitrobenzene as described in Liebigs Ann. Chem. 1992, 947-952. Each of these piperazine intermediates can be converted to compounds of the instant invention via chemistry depicted in Schemes 1-3 above.

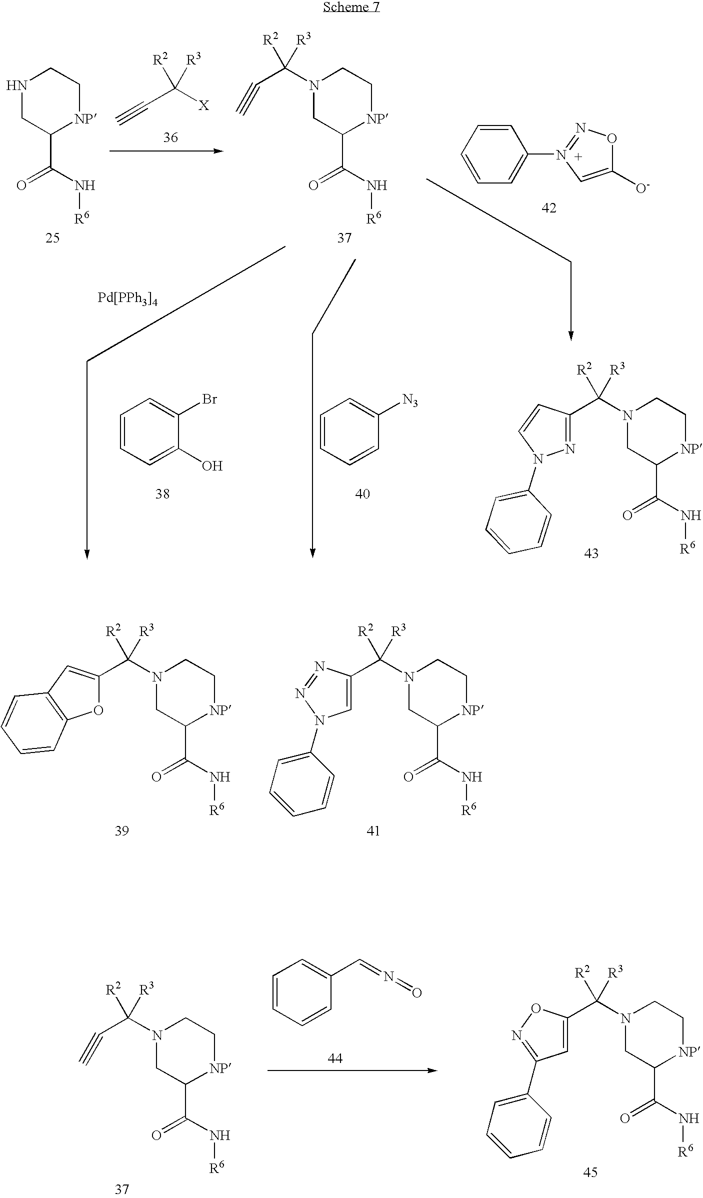

Oxazolyl piperazine intermediates such as 50 are available via the route shown in Scheme 8 below. Alkylation of piperazine 25 with bromo acid 46 in the presence of silver triflate in an inert solvent such as THF, according to methods detailed in J. Org. Chem. 1995, 60: 4013-4016, provides 47. Amide coupling of amine 48 to acid 47 to provide 49 can be carried out by any of the methods described above including the EDC/HOBT method. Amines such as 48 are prepared via chemistry described in Org. Synth. 1986, 64: 19-26 and Tetrahedron Letters 1999, 40: 6739-6743. Oxazole formation is accomplished by the action of a strong acid such as sulfuric acid on 49 in an inert solvent at elevated temperature, or as described in J. Med. Chem. 1996, 39: 2753-2763, to give intermediate 50. Again, intermediates such as these can be transformed into compounds of the instant invention via synthetic routes shown in Schemes 1,2, and 3.

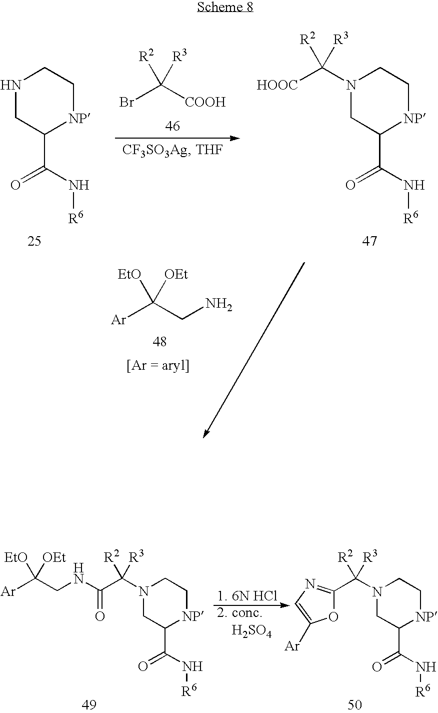

Further description on the preparation of compounds of Formula (I) is set forth in U.S. Ser. No. 09/718,223, filed Nov. 21, 2000, and in WO 01/38332, the disclosures of which are incorporated herein by reference in their entireties.

The following examples serve only to illustrate the invention and its practice. The examples are not to be construed as limitations on the scope or spirit of the invention.

### EXAMPLE 1

### Compound A Epoxide Intermediate

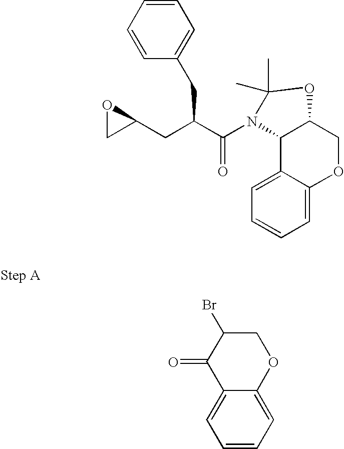

To a solution of 4-chromanone (10 g, 67.49 mmol) in 400 mL dichloromethane at 0° C. was added bromine (4.45 mL, 86.39 mmol) dropwise slowly. The reaction was monitored by TLC. After half an hour the reaction mixture was diluted with methylene chloride (100 mL) and was washed with water (300 mL). The organic layer was dried over anhydrous sodium sulfate and concentrated. The resulting product was dissolved in HOAc (100 mL) and sodium sulfite (8 g) was added. The reaction mixture was stirred at room temperature and reaction progress was monitored by TLC. After 48 hours the reaction mixture was poured into water and the product was extracted with methylene chloride. The organic layer was dried over anhydrous sodium sulfate and concentrated in vacuo to give the titled compound as a white solid. ¹H NMR (CDCl₃, 400 MHz): 7.93 (d, J=8.8 Hz, 1H), 7.54 (t, 1H), 7.08 (t, 1H), 7.02 (d, J=8.0 Hz, 1H), 4.63 (m, 4H)

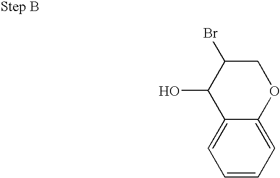

To a solution of 3-bromo-4-chromanone (2 g, 8.81 mmol) in methanol (20 mL) was added sodium borohydride (0.4 g, 10.57 mmol). The reaction mixture was stirred at room temperature and monitored by TLC. After 2 hours the solvent was removed in vacuo and then diluted with ethyl acetate (50 mL). The resulting solution was washed with brine, dried over anhydrous sodium sulfate, filtered and concentrated in vacuo to give the titled compound as a white solid. ¹H NMR (CDCl₃, 300 MHz): 7.32 (d, J=7.2 Hz, 1H), 7.23 (t, 1H), 6.96 (t, 1H), 6.84 (d, J=9.0 Hz, 1H), 4.82 (m, 1H), 4.54 (m, 1H), 4.38 (m, 2H).

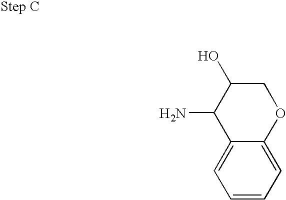

To a solution of 3-bromo-4-chromanol (2 g, 8.72 mmol) in acetonitrile (20 mL) was added concentrated sulfuric acid (1 mL, 17.47 mmol). The reaction mixture was stirred at 45° C.-50° C. for 18 hours. The solvent was removed in vacuo. Then water (10 mL) was added. The reaction mixture was heated to reflux. After 5 hours the reaction mixture was cooled to room temperature. The pH of the reaction mixture was adjusted to 12-13 by dropwise addition of aqueous 50% sodium hydroxide. The product was extracted with tetrahydrofuran three times. The organic layer were combined and dried over anhydrous sodium sulfate, filtered and concentrated in vacuo to give the title compound as a white solid. ¹H NMR (CDCl₃, 300 MHz): 7.29 (d, J=7.8 Hz, 1H), 7.16 (t, 1H), 6.93, (t, 1H), 6.83 (d, J=8.4 Hz, 1H), 4.12 (m, 1H), 3.99 (m, 2H), 3.84 (m, 1H).

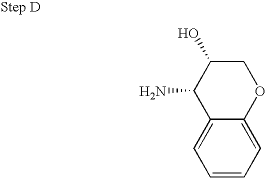

To a suspension of the racemic 4-amino-3-chromanol in ethanol (35 mL per gram of 4-amino-3-chromanol) was added 1.0 equivalent of (S)-(+) mandelic acid. The suspension was heated to 70° C. until forming a homogeneous solution. The solution was cooled to room temperature and white crystal was formed. After filtering the white crystal was dissolved in 3 N aqueous sodium hydroxide solution and the resolved product was extracted with ethyl acetate three times. The combined organic layer was dried over anhydrous sodium sulfate, filtered and concentrated in vacuo to give the titled compound as a white solid. The purity of the compound was verified by chiral HPLC with Crownpak CR+ column eluted with pH 1.0 perchloric acid solution. ¹H NMR (CDCl₃, 300 MHz): 7.29 (d, J=7.8 Hz, 1H), 7.16 (t, 1H), 6.93, (t, 1H), 6.83 (d, J=8.4 Hz, 1H), 4.12 (m, 1H), 3.99 (m, 2H), 3.84 (m, 1H).

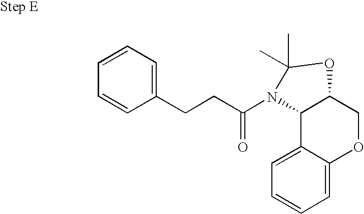

To a solution of the intermediate from Step D (5.97 g, 36.2 mmol) in THF (200 mL) was added hydrocinnamic acid (5.43 g, 36.2 mmol). The suspension was cooled to 0° C., and HOBT (5.23 g, 39.8 mmol) was added, followed by EDC (7.63 g, 39.8 mmol), and triethylamine (15.1 mL, 108 mmol). The mixture was warmed to ambient temperature and stirred 72 hours. The reaction mixture was poured onto 500 mL of 1.5 N aqueous HCl, and diluted with 200 mL of ethyl acetate. The organic layer was washed with an additional 200 mL of 1.5 N aqueous HCl, saturated aqueous NaHCO₃ (200 mL), and brine (200 mL), dried (MgSO₄), and concentrated in vacuo affording 16.0 g of a white solid. This material was dissolved in 400 mL of a 1:1 mixture of THF and 2,2-dimethoxypropane. To this solution was added 100 mg of p-toluenesulfonic acid, and the reaction was heated to reflux for 6 hours. The reaction was then cooled to ambient temperature and quenched by the addition of saturated aqueous NaHCO₃ (400 mL). The resulting mixture was extracted with ethyl acetate (400 mL×2). The organic layers were washed with brine (200 mL), dried (MgSO4), and concentrated in vacuo, affording 12.3 g of a yellow oil. Purification by flash chromatography (30% ethyl acetate in hexane) afforded the title compound as a white solid. ¹H NMR (CDCl₃, 300 MHz) 7.25 (m, 7H), 6.82 (m, 2H), 4.70 (d, 1H), 4.33 (m, 1H), 4.08 (d, 1H), 3.92 (s, 1H), 3.11 (m, 2H), 2.92 (m, 1H), 2.68 (m, 1H), 1.61 (s, 3H), 1.23 (s, 3H).

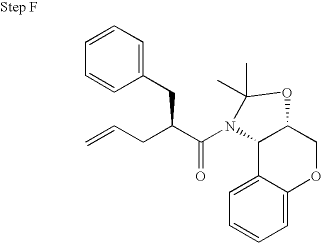

To a solution of the intermediate from Step E (6.36 g, 18.9 mmol) in THF (180 mL) was added allyl bromide (1.80 mL, 18.9 mmol). The solution was cooled to 22° C., and lithium hexamethyldisilylazide (20.8 mL of a 1.0 N solution in THF, 20.8 mmol) was added. After 10 min the reaction was quenched by the addition of saturated aqueous NH₄Cl (100 mL), and extracted with ethyl acetate (200 mL×2). The organic layers were washed with saturated aqueous NaHCO₃ (200 mL), brine (200 mL), dried (MgSO₄), and concentrated in vacuo. The resulting oil was purified by flash chromatography (25% ethyl acetate in hexane) affording the title compound as a white gum. ¹H NMR (CDCl₃, 300 MHz) indicated a 5:1 mixture of rotamers: 7.30 (m, 5H), 7.05 (m, 1H), 6.80 (m, 1H), 6.4 (m, 1H), 5.85 (m, 1H), 5.15 (m, 1H), 4.98 (m, 1H), 4.40 (m, 1H), 4.25 (m, 2H), 3.38 (dd, 1H), 3.19 (m, 1H), 2.80 (m, 1H), 2.42 (m, 1H), 1.70 (s, 3H), 1.23 (s, 3H).

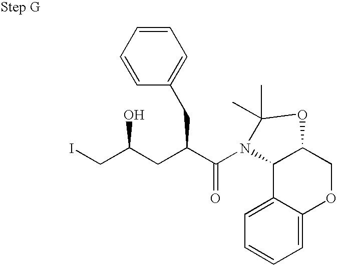

To a solution of the intermediate from Step F (6.10 g, 16.2 mmol) in 200 mL of ethyl acetate was added 200 mL of 0.5 aqueous NaHCO₃. The mixture was cooled to 0° C., and N-iodosuccinimide was added in a single portion. The reaction was warmed to ambient temperature and stirred 24 hr. The reaction was then diluted with ethyl acetate (500 mL). The organic layer was washed with 1N Na₂S₂O₃ (300 mL×2), and brine (300 mL), dried (MgSO₄), and concentrated in vacuo, affording a yellow oil. Purification by flash chromatography (30% ethyl acetate in hexane) afforded the title compound as a white solid. ¹H NMR (CDCl₃, 300 MHz) indicated a 5:2 mixture of rotamers: 7.30 (m, 5H), 7.05 (m, 1H), 6.82 (m, 1H), 6.60 (m, 1H), 5.92 (d, 0.3H), 5.58 (d, 0.7H), 4.45 (m, 2H), 4.20 (m, 2H), 3.63 (m, 1H), 3.44 (m, 2H), 3.20 (m, 2H), 2.82 (m, 2H), 2.40 (d, 1H), 2.00 (m, 1H), 1.72 (s, 3H), 1.49 (d, 2H), 1.29 (s, 3H).

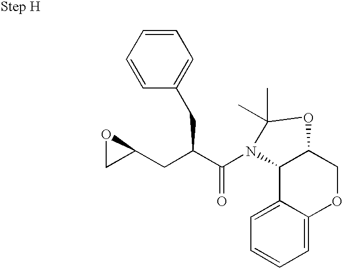

To a solution of the intermediate from Step G (7.71 g, 14.8 mmol) in ethyl acetate (300 mL) was added sodium methoxide (5.07 mL of a 25% solution in methanol, 22.2 mmol). After 10 minutes the reaction was quenched by the addition of saturated aqueous NaHCO₃ (300 mL). The organic layer was washed with brine (300 mL), dried (MgSO₄), and concentrated in vacuo affording the title compound as a white gum. This was used without further purification.

¹H NMR (CDCl₃, 300 MHz) indicated a 5:2 mixture of rotamers: 7.30 (m, 5H), 7.10 (m, 1H), 6.82 (m, 1H), 6.50 (m, 1H), 5.89 (d, 0.3H), 5.40 (d, 0.7H), 4.40 (m, 2H), 4.15 (m, 2H), 3.40 (m, 2H), 3.00 (m, 1H), 2.85 (m, 2H), 2.50 (dd, 0.7H), 2.40 (dd, 0.3H), 2.20 (m, 1H), 1.72 (s, 3H), 1.49 (d, 1H), 1.29 (s, 3H).

### EXAMPLE 2

(αR,γS,2S)-N-((3S,4S)-3,4-dihydro-3-hydroxy-2H-1-benzopyran-4-yl)-γ-hydroxy-α-(phenylmethyl)-2-[[(2,2,2-trifluoroethyl)amino]-carbonyl]-1-piperazinepentanamide

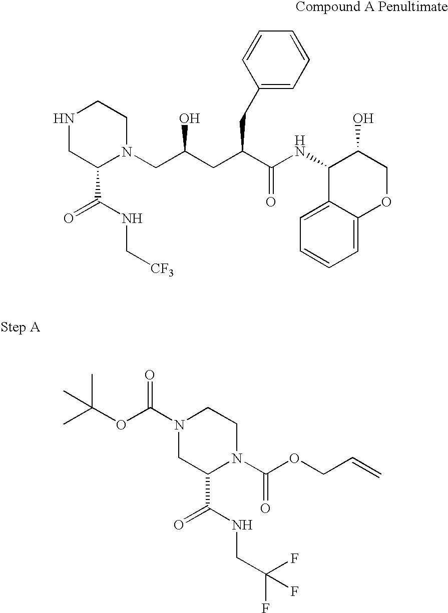

To a solution of 1,4-piperazine-2-(S)-carboxylic acid [bis(+)-CSA salt (30.0 g, 50.0 mmol) in 600 mL THF was added 1N aqueous NaOH until the resulting solution was pH 9 (150 mL). The solution was cooled to 0° C., and BOC-ON (12.3 g, 50.0) was added. The resulting solution was warmed to ambient temperature over 5 hours, then cooled again to 0° C. Allyl chloroformate (5.31 mL, 50.0 mmol) was added via syringe, followed by an additional 60 mL of 1N aqueous NaOH. The solution was warmed to ambient temperature overnight, then concentrated to minimum volume by rotary evaporator. The resulting mixture was acidified to pH 1 with 1N aqueous HCl, and extracted with ethyl acetate (400 mL×2). The organic layers were washed with brine (200 mL) dried (MgSO₄) and concentrated in vacuo, affording 23.7 g of a yellow oil. This material was dissolved in 750 mL of dichloromethane, followed by the addition of triethylamine (35.0 mL, 250 mmol), trifluoroethylamine (9.95 mL, 125 mmol), HOAT (10.2 g, 75.0 mmol), and EDC (14.4 g, 75.0 mmol). After 22 hours at ambient temperature the reaction mixture was quenched by the addition of saturated aqueous NaHCO₃ (500 mL). The organic layer was washed with an additional 500 mL of saturated aqueous NaHCO₃, then 1N aqueous NaHSO₄ (500 mL), and additional saturated aqueous NaHCO₃ (500 mL). The organic layer was dried (Na₂SO₄) and concentrated in vacuo. Purification by flash chromatography (40% ethyl acetate in hexane) afforded the title compound as a white solid. ¹H NMR (CDCl₃, 400 MHz) 5.95 (m, 1H), 5.35 (d, 1H), 5.28 (d, 1H), 4.75 (s, 1H), 4.68 (d, 1H), 4.53 (d, 1H), 3.90 (m, 3H), 3.20 (dd, 1H), 3.00 (m, 1H), 1.45 (s, 9H).

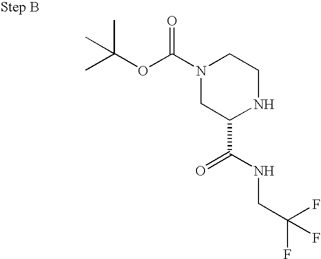

To a solution of tris (dibenzylidineacetone)dipalladium(0) (1.42 g, 1.55 mmol) in 150 mL of THF was added 1,4-bis(diphenylphosphino)butane (1.78 g, 3.10 mmol). After stirring 20 min at ambient temperature, this solution was added via cannula to a solution of the intermediate prepared in Step A (12.3 g, 31.0 mmol) and thiosalicilic acid (7.18 g, 46.6 mmol) in 150 mL of THF. After 1 hour at ambient temperature the reaction was diluted with 1 L of diethyl ether and extracted with 1% aqueous HCl (250 mL×3). The combined aqueous layers were neutralized with excess saturated NaHCO₃, and the resulting suspension was extracted with ethyl acetate (500 mL×2). These organic layers were washed with brine (200 mL), dried (MgSO₄), and concentrated in vacuo, affording the title compound as a clear oil. ¹H NMR (CDCl₃, 400 MHz) 7.28 (s, 1H), 4.00 (dd, 1H), 3.97 (m, 2H), 4.70 (s, 1H), 3.40 (dd, 1H), 3.20 (dd, 1H), 3.05 (s, 1H), 2.93 (d, 1H), 2.81 (t, 1H), 1.80 (s, 1H), 1.43 (s, 9H).

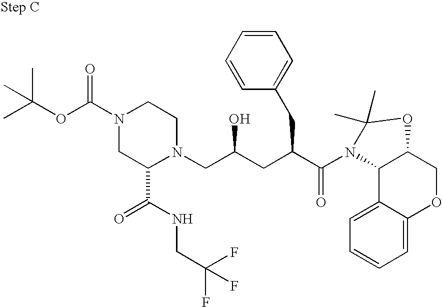

To a solution of the intermediate from Example 1, Step H (2.53 g, 6.45 mmol) in 2-propanol (30 mL) was added the intermediate from Step B (1.82 g, 5.86 mmol). The solution was heated to reflux for 7 hr, then cooled to ambient temperature and concentrated in vacuo, affording 2.82 g of a black oil. Purification by flash chromatography (65% ethyl acetate in hexane) afforded the title compound as a colorless oil. ¹H NMR (CDCl₃, 400 MHz) 7.25 (m, 5H), 7.20 (t, 1H), 7.18 (t, 1H), 7.15 (t, 1H), 7.03 (t, 1H), 6.83 (m, 1H), 6.60 (m, 2H), 5.89 (d, 1H), 5.50 (s, 1H), 4.45 (dd, 1H), 3.97 (dd, 1H), 4.23 (d, 1H), 4.00 (m, 1H), 3.82 (m, 2H), 3.68 (m, 1H), 3.45 (m, 3H), 3.32 (m, 3H), 2.87 (m, 1H), 2.67 (d, 1H), 2.50 (m, 2H), 1.82 (t, 1H), 1.76 (s, 3H), 1.74 (s, 3H), 1.42 (s, 9H), 1.24 (s, 6H); HPLC-MS (ES) 705.3 (M+1).

Step D:

(αR,γS,2S)-N-((3S,4S)-3,4-dihydro-3-hydroxy-2H-1-benzopyran-4-yl)-γ-hydroxy-α-(phenylmethyl)-2-[[(2,2,2-trifluoroethyl)amino]carbonyl]-1-piperazinepentanamide (Compound A Penultimate)

To a solution of the intermediate prepared in Step C (2.43 g, 3.45 mmol) in 2-propanol (20 mL) at 0° C. was added concentrated aqueous HCl (20 mL). After 16 hours at ambient temperature the reaction was brought to pH 8 with 2N aqueous NaOH. The mixture was then extracted with ethyl acetate (200 mL×2). The organic layers were washed with brine (200 mL), dried (MgSO₄), and concentrated in vacuo affording the title compound as a white solid. ¹H NMR (CDCl₃, 400 MHz) 9.05 (t, 1H), 7.28 (m, 5H), 7.13 (t, 1H), 7.10 (d, 1H), 6.80 (m, 2H), 6.20 (d, 1H), 5.20 (dd, 1H), 4.08 (m, 4H), 3.80 (m, 2H), 3.28 (s, 1H), 3.14 (m, 1H0, 2.98 (m, 4H), 2.65 (m, 2H), 2.48 (dd, 1H), 1.91 (t, 1H), 1.58 (t, 1H); HPLC-MS (ES) 565.2 (M+1).

### EXAMPLE 3

(αR,γS,2S)-4-[[5-(5-chloro-2-pyridinyl)-2-furanyl]methyl]-N-((3S,4S)-3,4-dihydro-3-hydroxy-2H-1-benzopyran-4-yl)-γ-hydroxy-α-(phenylmethyl)-2-[[(2,2,2-trifluoroethyl)amino]carbonyl]-1-piperazinepentanamide

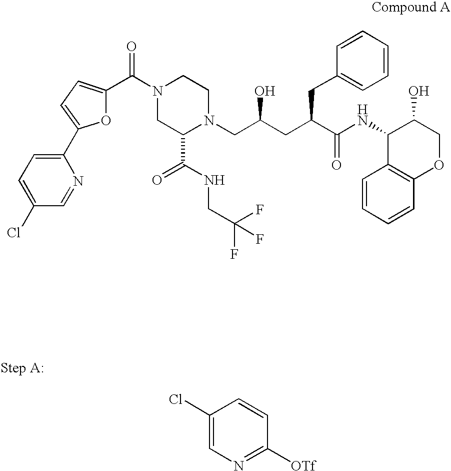

A suspension (60% wt) of NaH in mineral oil (340 mg; 8.47 mmol) was charged to a flask under nitrogen atmosphere and washed two times with dry THF. It was then suspended in dry THF (100 mL) and cooled to 0° C. A solution of 5-chloro-2-pyridinol (1.0 g; 7.7 mmol) in dry THF (100 mL) was added dropwise and the ice bath was removed. After 30 minutes, the reaction mixture was recooled to 0° C., neat CF₃SO₂Cl (0.902 mL; 8.47 mmol) was dripped in and again allowed to reach ambient temperature. Volatiles were removed in vacuo and the crude product was purified by Biotage flash chromatography (40M; 7% EtOAc/hexane) to provide desired compound as a pale yellow oil. ¹H-NMR (300 MHz, CDCl₃): δ7.16 (d, J=8.0 Hz, 1H), 7.86 (dd, J=8.6, 2.7 Hz, 1H), 8.35 (d, J=2.2 Hz, 1H).

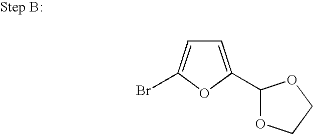

To a stirred solution of 5-bromo-2-furaldehyde (7.66 g; 43.8 mmol) in benzene (44 mL) was added ethylene glycol (6.02 mL; 109.5 mmol) and p-TsOH.H₂O (108 mg; 0.57 mmol). The reaction vessel was equipped with a Dean-Stark apparatus and heated to reflux for 75 minutes. The reaction mixture was poured in Et₂O (750 mL) and washed with saturated NaHCO₃ solution, water and brine. The organic layer was dried (MgSO₄), filtered, and concentrated in vacuo. The crude product was purified by flash column chromatography (gradient elution 4% to 5% EtOAc/hexane) to provide 9.1 g of the desired compound as a pale yellow oil (95% yield). ¹H-NMR (300 MHz, CDCl₃): δ3.98-4.15 (complex m, 4H), 5.87 (s, 1H), 6.28 (d, J=3.2 Hz, 1H), 6.41 (d, J=3.6 Hz, 1H).

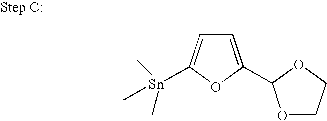

To a stirred solution of the intermediate from Step B (1.19 g; 5.43 mmol) in dry THF (29 mL) cooled to −78° C. was added dropwise t-BuLi (6.7 mL; 11.4 mmol). After 30 minutes a solution of trimethyltin chloride (1.19 g; 5.97 mmol) in dry THF (3 mL) was added dropwise. The reaction was allowed to warm to ambient temperature over 40 minutes. The volatiles were removed in vacuo and the residue was poured in Et₂O (200 mL), washed with saturated NaHCO₃, water, and brine, dried (Na₂SO₄), filtered, and concentrated in vacuo to provide the stannane which was used without further purification. ¹H-NMR (300 MHz, CDCl₃): δ0.32 (s, 9H), 4.00-4.14 (complex m, 4H), 5.98 (s, 1H), 6.45 (d, J=3.0 Hz, 1H), 6.52 (d, J=3.0Hz, 1H), 6.52 (d, J=3.2Hz, 1H).

Step D:

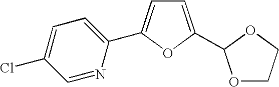

To a stirred solution of intermediate prepared in Step A (814 mg; 3.11 mmol) in dry DMF (31 mL) under nitrogen was added Pd(PPh₃)₄ (108 mg; 0.093 mmol) followed by AgO (385 mg; 3.11 mmol). After the mixture was stirred at 100° C. for 5 minutes, a solution of the stannane prepared in Step C in dry DMF (3 mL) was added. After an additional 10 minutes the mixture was cooled to room temperature, filtered through celite, and diluted with EtOAc (400 mL). After washing successively with saturated NaHCO₃ solution, water and brine, drying (Na₂SO₄), filtration, and removal of solvents in vacuo, the residue was purified by Biotage column chromatography (40S, 15% EtOAc/hexane) provided the bi-heteroaryl. ¹H-NMR (300 MHz, CDCl₃): δ4.04-4.16 (complex m, 4H), 6.00 (s, 1H), 6.57 (d, J=3.4 Hz, 1H), 7.01 (d, J=3.4 Hz, 1H), 7.66 (s, 1H), 8.51 (s, 1H).

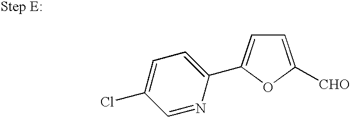

Route 1:

To a solution of intermediate prepared in Step D 685 mg;2.72 mmol) dissolved in THF (20 mL) was added 1N HCl (10.9 mL; 10.9 mmol). After 75 minutes the solution was brought to basic pH by the addition of dilute NH₄OH. THF was removed in vacuo and the residue was poured into EtOAc/Et₂O (400 mL). After washing successively with saturated NaHCO₃ solution, water and brine, drying (Na₂SO₄), filtration, and removal of solvents in vacuo, the desired aldehyde was obtained as a pale yellow solid and was used without further purification. ¹H-NMR (400 MHz, CDCl₃): δ7.26 (d, J=3.7 Hz, 1H), 7.37 (d, J=3.7 Hz, 1H), 7.78 (dd, J=8.4, 2.3 Hz, 1H), 7.90 (d, J=8.4 Hz, 1H), 8.61 (d, J=2.5 Hz, 1H), 9.73 (s, 1H).

Route 2:

The aldehyde was alternatively also prepared as follows:

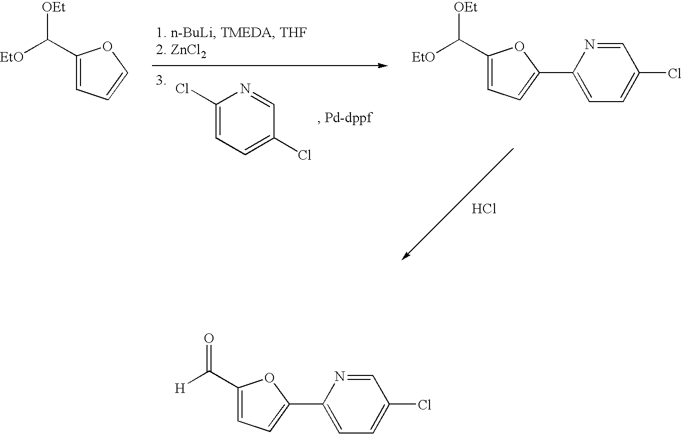

THF (125 mL; KF<200 ppm), TMEDA (24.40 mL; 1.1 eq.; KF<125 ppm) and 2-furaldehyde diethyl acetal (24.80 mL) were added at room temperature to a 1L round bottomed flask equipped with a thermocouple, an overhead stirrer, N₂ inlet and an addition funnel. The solution. was cooled to −40° C. over 15 min., and then n-BuLi (101 mL; 1.1 eq.) was added over 1 hour with the temperature maintained at less than −20° C. The mixture was stirred 15 min at −25 ° C., and then assayed via LC. The assay showed 96% deprotonation. The reaction mixture was then cooled to −35° C., and a slurry of 1.5M ZnC₂/THF (68.5 mL; 0.7 eq.; KF=680 ppm-dried by soxhlet distillation through molecular sieves for 3 days) was added over 1 h while maintaining the temperature at less than <−20° C. throughout the addition. The mixture was then stirred for 30 min at −25° C. and warmed to 25° C. over 60 min. Solid Pd(dppf)Cl₂ (0.60 g; 0.5 mol %) was then added, followed by solid 2,5-dichloropyridine (23.91 g; 1.1 eq.), each in one portion. The mixture was then heated to 55° C. and aged for 3 h (95% conversion by NMR assay; ˜85% assay yield by LC), after which the mixture was allowed to cool to room temperature and stirred overnight.

The reaction mixture was then cooled to 0° C. and quenched with 5° C. 5M AcOH (294 mL; 5 eq.) over 10 min with the temp. maintained less than 25° C. throughout. The mixture was agitated for 15 min at 23° C. and then allowed to settle for 2 h. The aqueous layer was removed and the organic layer was-cooled to 0° C., followed by addition thereto of 5° C. 10% NaOH (250 mL; 5 mL/g) over 10 min with the temperature maintained <25° C. throughout. The mixture was agitated for 15 min at 23° C., allowed to settle for 2 h, the aqueous layer removed, followed by addition of sat'd brine (62.5 mL; 2.5 mL/g) over 2 min with the temp. maintained less than 25° C. The mixture was agitated for 15 min at 23° C., allowed to settle for 2 h, and the aqueous layer removed.

The organic soln. was concentrated down to 5 mL/g (125 mL) under vacuum with the soln.'s temperature maintained between 25-35° C. The concentrated solution was then diluted to 10 mL/g (250 mL) with heptane. This was repeated twice more to solvent switch completely to heptane (THF<1%). Darco G-60 (12.5 g) was added to the solution, and the mixture was heated to 50° C. for 2 h, cooled to 23° C. over 1 h and aged at 23° C. for 15 h. The mixture was then filtered through solka floc (25 g) and the filtercake was washed with heptane (250 mL).

The heptane solution of the acetal was then added to a 500 mL round bottomed flask equipped with a thermocouple, an overhead stirrer, an N₂ inlet and a distillation setup, concentrated down to 340 mL, and then diluted up with THF (25 mL). One quarter of an acid charge consisting of HCl (5M; 3 mL=10 mol % based on starting acetal) diluted in 12.5 mL of THF was added to the acetal soln. over 1 min and aged for 5 min at room temperature. The batch was then seeded with aldehyde 0.25 g and aged at room temperature for 15 min upon which some of the aldehyde began to crystallize out. The remaining acid charge was then added over 5 min and the slurry was aged at room temperature for 2 h. After such time, the deprotection was only 90% complete as determined by LC assay, so an additional 0.3 mL of acid was added to the slurry. The slurry was aged for an additional 30 min with little change in the percentage of deprotected aldehyde.

The slurry was constant volume batch concentrated at ˜350 mL with 200 mL of heptane being flushed through to remove the THF and the EtOH which formed upon deprotection. (The temperature of the slurry was maintained <35° C.). The slurry was diluted to 375 mL with heptane and cooled to 23° C. The deprotection was complete at this time, with only about 1% acetal remaining. The solid aldehyde was filtered and displacement washed with 250 mL of r.t. heptane and dried overnight under a stream of nitrogen. The aldehyde was then dried for 2 days at 40° C. and 200 torr.

Step F:

(αR,γS,2S)-4-[[5-(5-chloro-2-pyridinyl)-2-furanyl]methyl]-N-((3S,4S)-3,4-dihydro-3-hydroxy-2H-1-benzopyran-4-yl)-γ-hydroxy-α-(phenylmethyl)-2-[[(2,2,2-trifluoroethyl)amino]carbonyl]-1-piperazinepentanamide (Compound A)

To a solution of the aldehyde obtained from Step E, Route 1 above (42 mg; 0.200 mmol) and penultimate intermediate from Example 2, Step D (75 mg; 0.133 mmol) in anhydrous DMF (1.2 mL) was added NaHB(OAc)₃ (43 mg; 0.200 mmol). After 18 hours the solution was poured into EtOAc, washed with saturated NaHCO₃ solution, water and brine, dried (Na₂SO₄), filtered, and solvent removed in vacuo. Purification by Biotage column chromatography (12M; 4% MeOH/CH₂Cl₂) provided the desired compound as a white solid. ¹H-NMR (400 MHz, CD₃OD): δ1.39 (m, 1H), 2.06 (m, 1H), 2.33-2.46 (complex m, 3H), 2.49-2.55 (m, 1H), 2.57-2.62 (m, 1H), 2.72-2.82 (complex m, 3H), 2.91-3.06 (complex m, 3H), 3.10 (dd, J=3.3, 8.0 Hz, 1H), 3.69 (s, 2H), 3.71-3.80 (complex m, 3H), 3.94-4.02 (complex m, 1H), 4.04-4.08 (complex m, 2H), 5.15 (d, J=4.1 Hz, 1H), 6.48 (d, J=3.3 Hz, 1H), 6.73 (dd, J=1.2, 8.2 Hz, 1H), 6.82 (apparent td, J=1.2, 7.5 Hz, 1H), 7.06 (d, J=3.3 Hz, 1H), 7.07-7.28 (complex m, 7H), 7.74 (dd, J=0.8, 8.6 Hz, 1H), 7.86 (dd, J=2.5, 8.6 Hz, 1H), 8.49 (m, 1H); electrospray ionization mass spectrum: m/e 756.4 (MH⁺ calcd for C₃₈H₄₁ClF₃N₅O₆, 756.3).

### EXAMPLE 4

Preparation of 4-(tert-butyloxycarbonyl)-2(S)-((2,2,2-trifluoroethyl)aminocarbonyl) piperazine

Step One:

Preparation of the pyrazine amide

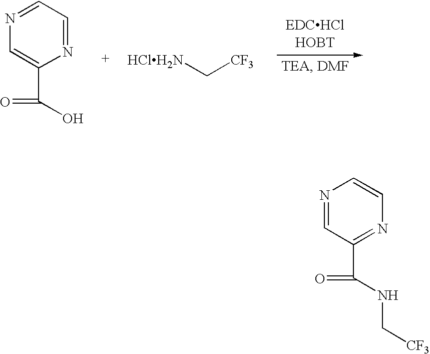

Pyrazine 2-carboxylic acid (1204 g) was suspended in DMF (4.8 L, 4 mL/g acid). 2,2,2-trifluoroethylamine.HCl (TFEA.HCl) (1200 g), 1-hydroxybenzotriazole (HOBT) (60 g) and triethylamine (TEA) (1410 mL) were then added sequentially (exotherm upon addition of TEA, flask cooled with ice bath and temperature kept below 35° C.). The reaction was cooled to 15° C. and 1-(3-dimethylaminopropyl)-3-ethylcarbodiimide.HCl (EDC.HCl) (1940 g) was added portionwise over 15-30 min. The reaction temperature was kept below 35° C. When the reaction appeared complete (approx. two hours, <5% pyrazine 2-carboxylic acid by LC assay), the reaction mixture (yellow/white slurry) was diluted with 10% K₂CO₃ in water (24 L, 20 mL/g acid) and the reaction slurry was kept below 35° C. The slurry was cooled to 10° C., aged for two hours and filtered (mother liquor assay=3-4 mg/mL). The wet cake was washed with deionized water (12 L, 10 mL/g acid) and dried under vacuum (22″ Hg) at 40° C. with a nitrogen purge. Theoretical yield of 1816 g . Actual yield 1533 g (84%).

¹H NMR: (CD₃CN, 400 MHz): δ9.29(d, J=1.5 Hz, 1H), 8.82 (d, J=2.5 Hz, 1H), 8.63 (dd, J=2.6,1.4 Hz, 1H), 8.40 (bs, 1H), 4.14 (dq, J=9.4, 6.8 Hz, 2H).

HPLC Assay conditions: Waters Xterra RP8 column, elution with acetonitrile and 5 mM K phosphate adjusted to pH=8, detection at 220 nm.

Step Two:

Preparation of the piperazine amide

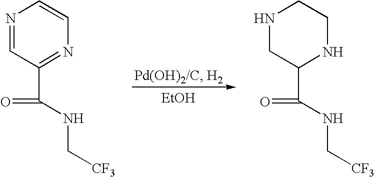

Pyrazine amide (60.2 g 0.268 mol, not corrected for water content) was suspended in absolute ethanol (550 mL) in a 1.0 L autoclave hydrogenation vessel and cooled to 15° C. Wet 20% Pd(OH)₂/C 11.0 g (20 wt %, 50wt % wet) was added and reaction was purged with N₂ three times. H₂ (5 psig) was introduced with stirring and the temperature maintained at 15° C. for 60 minutes. The temperature was then increased to 60° C. and the hydrogen pressure increased to 40 psig and the reaction mixture stirred for 18 additional hours. The reaction was considered complete when conversion is >99% by LC assay. The reaction mixture was filtered through Solka-Floc and the catalyst solids were washed with ethanol 2×110 mL. Assay of the combined filtrate and washes gave 53.5 g of racemic piperazine amide (Yield=86%)

¹H NMR (CD₃CN, 400 MHz): δ7.58 (bs, 1H), 3.90 (dq, J=9.5,6.7 Hz, 2H), 3.24(dd, J=7.9, 5.5 Hz, 1H), 2.96 (dd, J=12.1, 3.6 Hz, 1H), 2.84-2.78 (m, 1H), 2.77-2.67 (m, 3H), 2.66-2.56 (m, 1H), 1.90 (s, 2 H).

HPLC Assay conditions: YMC Basic column, elution with acetonitrile and 0.1% aqueous H₃PO₄, detection at 210 nm.

Step Three:

Resolution of the piperazine amide

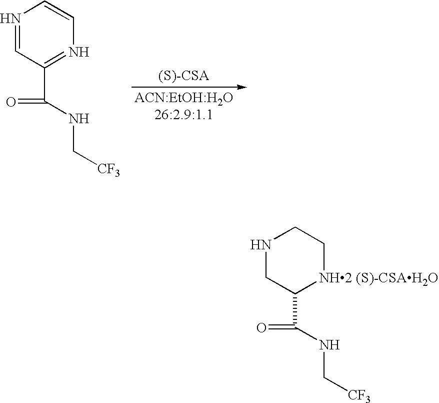

The pip amide ethanol filtrate (116.37 g containing 10.3 g of racemic pip amide by LC assay) was concentrated in vacuo to a final volume of 40.2 mL (3.9 mL per gram of pip amide) and the slurry is diluted with 82.4 mL (8 mL per gram pip amide) of acetonitrile (ACN) and stirred until homogenous. Separately (S)-camphorsulfonic acid ((S)-CSA) (19.26 g, MW=232.30, 1.7 eq) was dissolved in 185 mL of ACN (18 mL per gram of pip amide). The water content of the two solutions was then determined by Karl Fisher titration. The CSA solution was added to the pip amide solution giving a small exotherm to approx. 31-32° C. Water (11.02 mL, 1.118 mL per gram of pip amide minus the total water content of the two solutions) was then added, such that the acetonitrile:ethanol:water ratio was 26:2.9:1.1 (v/v/v). Solids began to form after 15-30 min. The solution/slurry was heated to 72° C. to completely dissolve all solids. The yellow solution was recooled to 62° C. and seeded with a slurry of 10.3 mg of pip amide salt in 1 mL of acetonitrile. After a two hour age at 62° C. the slurry was allowed to cool to room temperature overnight (crystallization was complete when loss to mother liquors was <21 mg pip amide/mL by LC assay. The slurry was filtered then washed with 2×30 mL of ACN:EtOH:H₂O [(26:2.9:1.1), (v:v:v)] solution. The wet cake (˜13 g, white solid) was dried at 40° C. in a vacuum oven (24 in Hg, nitrogen sweep) to give 11.16 g of product (yield=33%). Assay method (Pip Amide) as above. Chiral assay gives an enantiomeric excess (ee) of 98.0%.

¹H NMR (CD₃OD, 400 MHz): d4.84(bs, 5H), 4.64 (dd, J=12.0, 3.6 Hz, 1H), 4.13-3.9 (m, 3H), 3.77 (m, 2H), 3.66 (m, 1H), 3.54-3.43 (m, 2H), 3.28(d, J=14.7 Hz, 2H), 2.82 (d, 14.7 Hz, 2H), 2.55 (m, 2H), 2.36 (m, 2H), 2.12-1.998 (m, 4H), 1.92 (d, J=18.4 Hz, 2H), 1.72 (m, 2H), 1.45 (m, 2H), 1.09 (s, 6H), 0.87 (s, 6H).

Enantiomeric excess determined by chiral HPLC of the mono BOC piperazine amide. HPLC assay conditions: Chiral AGP column, elution with acetonitrile and 10 mM Kphospate, pH=6.5, detection at 210 nm.

Step Four:

Upgrade of ee of (S)-piperazine amide bis (S)-CSA salt

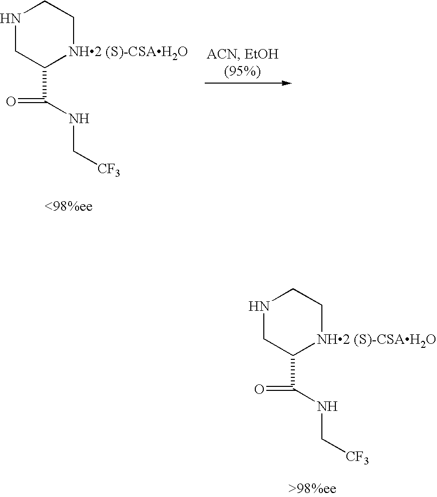

To a 12 L flask was charged (S)-pip amide salt (412.87 g) having an ee of less than 98%, 7.43 L of ACN and 825 mL of 190 proof EtOH. The slurry was heated to 75° C., aged for 1 hr at 75° C. (during heating the slurry thickened considerably), then allowed to cool to 25° C. overnight. The slurry was filtered and washed with EtOH (190 proof):ACN (10:90) (2×800 mL, 2 mL/g). The white solid was dried in a vacuum oven at 24 in Hg, 40° C. with a nitrogen sweep to give 400 g of product with an ee of 99%. Assays (normal and chiral) were performed as described above in the prior steps.

Step Five:

Procedure for (S)-Mono BOC piperazine amide: BOC Protection

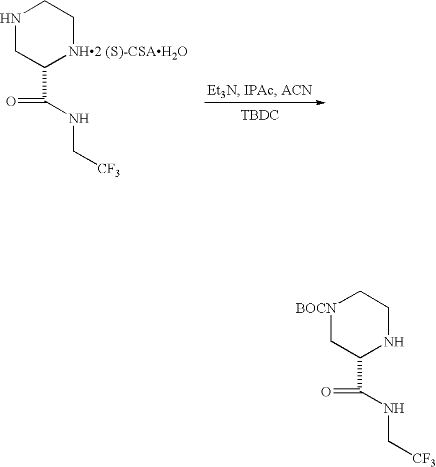

Bis (S)-CSA piperazine amide salt (20 g) was suspended in a mixture of 113 mL of isopropyl acetate (IPAc) and 57 mL of acetonitrile. Triethylamine (8.26 mL, 2 eq) was added and the mixture stirred until homogenous. A solution of TBDC (6.46 g, 1.0 eq) in a mixture of 20 mL isopropyl acetate and 10 mL of ACN was then added over 10 minutes. After aging for two hours the solution was assayed as necessary by LC (Pip Amide Assay, see above) until the reaction was complete (i.e., less than 5% starting material). When the reaction was complete, 100 mL of water and 135 mL of isopropyl acetate were added, the resulting layers were separated and the organic layer was concentrated to 28 mL. The residue was then diluted with 28 mL of isopropyl alcohol and reconcentrated to 28 mL. This was repeated two additional times. The yield of BOC pip amide was 87% with a mono:bis BOC ratio of 95:5, as determined by HPLC.

¹H NMR (CDCl₃, 400MHz): δ=7.39 (app t, J=6.3 Hz, 1H), 3.96 (dd, J=3.5, 13.4 Hz, 1H), 3.88 (m, 2H), 3.67 (d, J=11.5 Hz, 1H), 3.39 (dd, J=3.8, 8.6 Hz, 1H), 3.13 (dd, J=8.6, 13.3 Hz, 1H), 3.02 (br, 1H), 2.91 (m, 1H), 2.77 (m, 1H), 1.43 (s, 9H).

¹³C NMR (CDCl₃,) δ=171.43, 154.41, 123.89 (q, J=78.5 Hz), 80.16, 57.65, 43.63, 45.6 (br), 44.0 (br), 40.20 (q, J=34.7 Hz), 28.19.

HPLC Assay conditions: YMC Basic column, elution with acetonitrile and 0.1% aqueous H₃PO₄, detection at 210 nm. The BOC piperazine amide of Step 5 can be reacted with the intermediate of Example 1, Step H in the manner described in Example 2, Step C to obtain the title compound of Example 2, Step C, and ultimately Compound A penultimate.

### EXAMPLE 5

### Preparation of Cation Exchange Resin-Compound A Complex

Amberlite® IRP-69 ion exchange resin (4.15 g) and ethanol (200 mL) were added to a 500 mL Erlenmeyer flask containing Compound A (2.015 g; white powder that had been milled to a mean particle size of 2.7 μm (with 95% of the particles having a particle size of less than 4.2 μm) using a Jet-O-Mizer Modell 00 jet mill manufactured by Fluid Energy Aljet (Plumsteadville, Pa.), followed by the addition of 2.7 mL of 1.0N HCl (1.0 eqivalent per mole of Compound A) while stirring the mixture at room temperature with a Teflon-coated stir bar. An additional 0.27 mL of 1.0 N HCl was subsequently added in order to complete the dissolution of Compound A. Additional ethanol (100 mL) was then added, and the solution was stirred at room temperature for thirty minutes. The contents of the Erlenmeyer were then transferred to a round bottomed flask and the solvent was removed by rotary evaporation to afford a slightly brown solid, which was subsequently washed with water to remove HCl and unbound compound.

### EXAMPLE 6

### Pharmacokinetic Study in Dogs

Four samples of the resin complex prepared in Example 5, each sample weighing about 246 mg (about 70 mg of Compound A), were respectively placed into four OSC size capsules (Capsugel®, Warner-Lambert), which were then closed. Four Beagle dogs were dosed orally with the capsules which provided 10 mg of Compound A per kg of body weight (mpk) followed by 5 mL/kg of water. The dogs were pre-acidified with a subcutaneous injection of pentagastrin about 10 to 15 minutes prior to dosing the capsules. The dose of pentagastrin was variable due to adverse reactions in the dogs, but each dog received enough pentagastrin to lower the stomach pH. The same dogs were also dosed orally with a control capsule.

The same dogs (without pre-acidification) were also orally dosed with 10 mpk of (i) Compound A dissolved in propylene glycol-HCl-ethanol (98:2:5) and (ii) Compound A in an encapsulated polyethylene glycol-HCl gel. For each dosing run, blood samples were drawn from catheters placed in the cephalic vein at pre-dose at 0, 0.333, 0.5, 0.67, 1, 2, 4, 6, 8 and 24 hours after dosing. The sample extracts were extracted via a liquid-liquid extraction procedure employing methyl t-butyl ether to isolate Compound A from the biological mixture/plasma. Indinavir was used as the internal standard. The sample extracts were analyzed by LC/MS/MS in the positive ion mode using an electrospray interface. The area under the curve (AUC) was calculated by linear trapezoidal rule from observed data points using Microsoft Excel 97 SR-2. Arithmetic mean and standard error of the mean (SEM) for AUC, observed maximum plasma concentration (Cₘₐₓ), and time of Cₘₐₓ (Tₘₐₓ) were calculated with Microsoft Excel v 97 SR-2. The results are shown in Tables 2, 3 and 4 below.

TABLE 2
AUC₀₋₂₄ hrs (μM · hr)

| | Resin Complex | Resin Capsule | Glycol | Polyglycol |
|---|---|---|---|---|
| | Capsule | Control | Solution | Capsule |
| Dog 1 | 0.4735 | 0.0156 | 0.8245 | 1.523 |
| Dog 2 | 0.1614 | 0.2022 | 0.1364 | 0.305 |
| Dog 3 | 0.2884 | 0.0218 | 0.0536 | 0.055 |
| Dog 4 | 0.7104 | 0.0601 | 0.3788 | 0.217 |
| Mean | 0.4084 | 0.0749 | 0.3483 | 0.5251 |
| SEM | 0.1193 | 0.0436 | 0.1731 | 0.3367 |

TABLE 3
Cₘₐₓ (μM)

| | Resin Complex | Resin Capsule | Glycol | Polyglycol |
|---|---|---|---|---|
| | Capsule | Control | Solution | Capsule |
| Dog 1 | 0.258 | 0.0071 | 0.595 | 1.32 |
| Dog 2 | 0.0975 | 0.142 | 0.115 | 0.275 |
| Dog 3 | 0.244 | 0.0126 | 0.0729 | 0.031 |
| Dog 4 | 0.513 | 0.062 | 0.435 | 0.144 |
| Mean | 0.278 | 0.056 | 0.305 | 0.444 |
| SEM | 0.086 | 0.031 | 0.126 | 0.298 |

TABLE 4
Tₘₐₓ (hr)

| | Resin Complex | Resin Capsule | Glycol | Polyglycol |
|---|---|---|---|---|
| | Capsule | Control | Solution | Capsule |
| Dog 1 | 1.00 | 1.00 | 0.50 | 0.50 |
| Dog 2 | 1.00 | 0.75 | 0.25 | 0.50 |
| Dog 3 | 1.00 | 1.00 | 0.25 | 1.00 |
| Dog 4 | 1.00 | 0.50 | 0.50 | 0.75 |
| Mean | 1.00 | 0.81 | 0.38 | 0.69 |
| SEM | 0.00 | 0.12 | 0.07 | 0.12 |

Tables 2-4 show that the Compound A-resin complex with pre-acidification has a pharmacokinetic profile similar to that of the glycol solution and the polyglycol gel capsule. An advantage of the resin complex over the solution and the gel formulations is that it is a solid which can facilitate handling and administration. Another advantage is that the resin complex is more stable thermally. Thermal stability studies conducted at 25° C. and 40° C. on these three formulations have indicated that the Compound A-resin complex has little or no growth of degradates (e.g., lactones) relative to the solution and gel formulations. The resin complex does require an acidic stomach in order to release the drug, but this can routinely and easily be accomplished by administering the complex to the subject after a meal or with a carbonated beverage.

While the foregoing specification teaches the principles of the present invention, with examples provided for the purpose of illustration, the practice of the invention encompasses all of the usual variations, adaptations and/or modifications that come within the scope of the following claims.

What is claimed is:

1. A pharmaceutical composition which comprises a cation exchange resin complexed with a compound of Formula (I) or a pharmaceutically acceptable salt thereof: 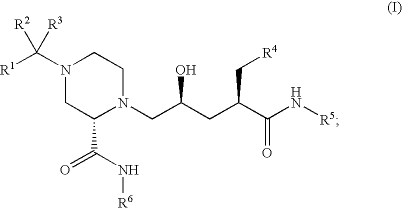
wherein
R¹ is C₁-C₆ alkyl, C₂-C₆ alkenyl, C₂-C₆ alkynyl, C₃-C₆ cycloalkyl, aryl, substituted aryl, heteroaryl, or substituted heteroaryl; wherein
(i) each of the substituents on substituted aryl is independently
(a) halogen,
(b) cyano,
(c) hydroxy,
(d) C₁-C₆ alkyl,
(e) C₂-C₆ alkenyl,
(f) C₂-C₆ alkynyl,
(g) fluorinated C₁-C₆ alkyl,
(h) C₁-C₆ alkoxy,
(i) fluorinated C₁-C₆ alkoxy,
(j) S-(C₁-C₆ alkyl),
(k) heterocycle, or
(l) heterocycle substituted with one or more substituents independently selected from halogen, cyano, hydroxy, C₁-C₆ alkyl, C₂-C₆ alkenyl, C₂-C₆ alkynyl, fluorinated C₁-C₆ alkyl, C₁-C₆ alkoxy, fluorinated C₁-C₆ alkoxy, S-(C₁-C₆ alkyl), and NR^{a}R^{b};
(ii) each of the substituents on substituted heteroaryl is independently
(a) halogen,
(b) cyano,
(c) hydroxy,
(d) NR^{a}R^{b},
(e) C₁-C₆ alkyl,
(f) C₂-C₆ alkenyl,
(g) C₂-C₆ alkynyl,
(h) fluorinated C₁-C₆ alkyl,
(i) C₁-C₆ alkoxy,
(j) fluorinated C₁-C₆ alkoxy,
(k) S-(C₁-C₆ alkyl),
(l) phenyl,
(m) phenyl substituted with one or more substituents independently selected from halogen, cyano, hydroxy, C₁-C₆ alkyl, C₂-C₆ alkenyl, C₂-C₆ alkynyl, fluorinated C₁-C₆ alkyl, C₁-C₆ alkoxy, fluorinated C₁-C₆ alkoxy, and S-(C₁-C₆ alkyl),
(l) heterocycle, or
(m) heterocycle substituted with one or more substituents independently selected from halogen, cyano, hydroxy, C₁-C₆ alkyl, C₂-C₆ alkenyl, C₂-C₆ alkynyl, fluorinated C₁-C₆ alkyl, C₁-C₆ alkoxy, fluorinated C₁-C₆ alkoxy, S-(C₁-C₆ alkyl), NR^{a}R^{b}, and a 5- or 6-membered heteroaromatic ring consisting of carbon atoms and from 1 to 3 heteroatoms selected from N, O and S;
R² and R³ are each independently hydrogen or C₁-C₄ alkyl; or R² and R³ together with the carbon to which they are attached form C₃-C₆ cycloalkyl;
R⁴ is C₁-C₆ alkyl, C₃-C₆ cycloalkyl, aryl, substituted aryl, heteroaryl, or substituted heteroaryl; wherein each of the substituents on substituted aryl is independently halogen, hydroxy, C₁-C₆ alkyl, C₂-C₆ alkenyl, C₂-C₆ alkynyl, fluorinated C₁-C₆ alkyl, C₁-C₆ alkoxy, or heteroaryl; and each of the substituents on substituted heteroaryl is independently halogen, hydroxy, cyano, C₁-C₆ alkyl, C₂-C₆ alkenyl, C₂-C₆ alkynyl, fluorinated C₁-C₆ alkyl, C₁-C₆ alkoxy, or aryl;
R⁵ is carbocyclic, substituted carbocyclic, heterocyclic or substituted heterocyclic, wherein each of the substituents on substituted carbocyclic or substituted heterocyclic is independently halogen, hydroxy, C₁-C₆ alkyl, C₂-C₆ alkenyl, C₂-C₆ alkynyl, fluorinated C₁-C₆ alkyl, or C₁-C₆ alkoxy;
R⁶ is fluorinated C₁-C₆ alkyl; and
R^{a} and R^{b} are each independently hydrogen or C₁-C₄ alkyl; or R^{a} and R^{b} together with the nitrogen to which they are attached form C₃-C₆ azacycloalkyl.

2. The composition according to claim 1, wherein the complex comprises a compound of Formula (I) or a pharmaceutically acceptable salt thereof, wherein:
R¹ is C₁-C₆ alkyl, C₃-C₆ cycloalkyl, aryl, substituted aryl, heteroaryl, or substituted heteroaryl, wherein heteroaryl is (i) a 5- or 6-membered aromatic ring consisting of carbon atoms and from 1 to 3 heteroatoms selected from N, S and O or (ii) an 8- to 10-membered bicyclic ring system consisting of carbon atoms and from 1 to 3 heteroatoms selected from N, S and O, wherein at least one of the rings in the bicyclic system is an aromatic ring; wherein
(i) each of the substituents on substituted aryl is independently
(a) halogen,
(b) cyano,
(c) hydroxy,
(d) C₁-C₆ alkyl,
(e) C₂-C₆ alkenyl,
(f) C₂-C₆ alkynyl,
(g) fluorinated C₁-C₆ alkyl,
(h) C₁-C₆ alkoxy,
(i) fluorinated C₁-C₆ alkoxy,
(j) S-(C₁-C₆ alkyl),
(k) heterocycle, or
(l) heterocycle substituted with one or more substituents independently selected from halogen, cyano, hydroxy, C₁-C₆ alkyl, C₂-C₆ alkenyl, C₂-C₆ alkynyl, fluorinated C₁-C₆ alkyl, C₁-C₆ alkoxy, fluorinated C₁-C₆ alkoxy, S-(C₁-C₆ alkyl), and NR^{a}R^{b}; and
(ii) each of the substituents on substituted heteroaryl is independently
(a) halogen,
(b) cyano,
(c) hydroxy,
(d) NR^{a}R^{b},
(e) C₁-C₆ alkyl,
(f) C₂-C₆ alkenyl,
(g) C₂-C₆ alkynyl,
(h) fluorinated C₁-C₆ alkyl,
(i) C₁-C₆ alkoxy,
(j) fluorinated C₁-C₆ alkoxy,
(k) S-(C₁-C₆ alkyl),
(l) phenyl,
(m) phenyl substituted with one or more substituents independently selected from halogen, cyano, hydroxy, C₁-C₆ alkyl, C₂-C₆ alkenyl, C₂-C₆ alkynyl, fluorinated C₁-C₆ alkyl, C₁-C₆ alkoxy, fluorinated C₁-C₆ alkoxy, and S-(C₁-C₆ alkyl),
(l) heterocycle, or
(m) heterocycle substituted with one or more substituents independently selected from halogen, cyano, hydroxy, C₁-C₆ alkyl, C₂-C₆ alkenyl, C₂-C₆ alkynyl, fluorinated C₁-C₆ alkyl, C₁-C₆ alkoxy, fluorinated C₁-C₆ alkoxy, S-(C₁-C₆ alkyl), NR^{a}R^{b}, and a 5- or 6-membered heteroaromatic ring consisting of carbon atoms and from 1 to 3 heteroatoms selected from N, O and S.

3. The composition according to claim 1, wherein the complex comprises a compound of Formula (I) or a pharmaceutically acceptable salt thereof, wherein:
R⁴ is 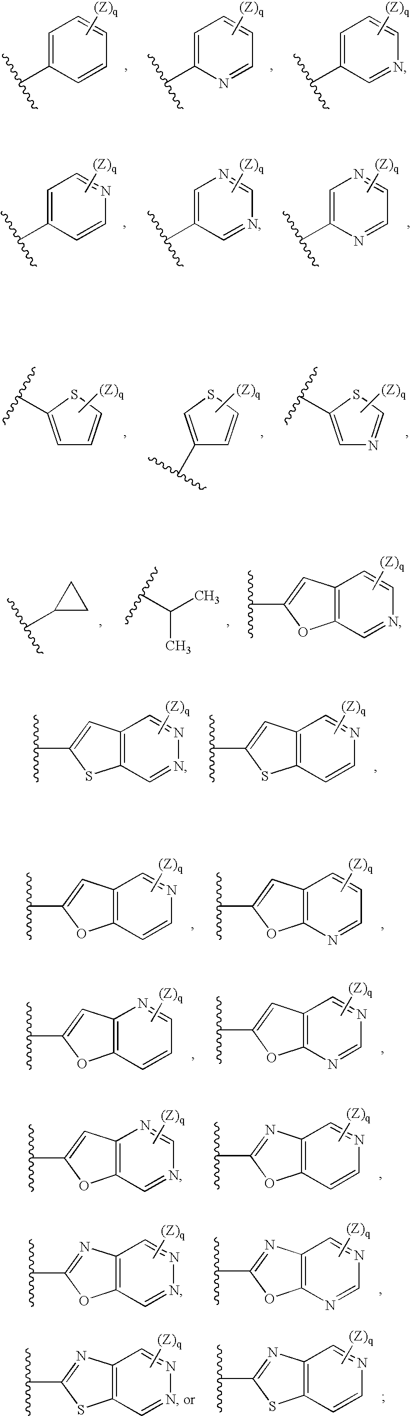
each Z is independently hydrogen, halogen, hydroxy, cyano, C₁-C₆ alkyl, C₁-C₆ fluroinated alkyl, or C₁-C₆ alkoxy; and
q is an integer from 0 to 2.

4. The composition according to claim 1, wherein the complex comprises a compound of Formula (I) or a pharmaceutically acceptable salt thereof, wherein:
R⁵ is carbocyclic, substituted carbocyclic, heterocyclic or substituted heterocyclic, wherein carbocyclic is cyclopentyl, indanyl, or tetralin, and heterocyclic is chroman, thiochroman, or dioxoisothiochroman; wherein each of the substituents on substituted carbocyclic or substituted heterocyclic is independently halogen, hydroxy, C₁-C₆ alkyl, fluorinated C₁-C₆ alkyl, or C₁-C₆ alkoxy.

5. The composition according to claim 1, wherein the complex comprises a compound of Formula (I), or a pharmaceutically acceptable salt thereof, wherein:
R⁶ is

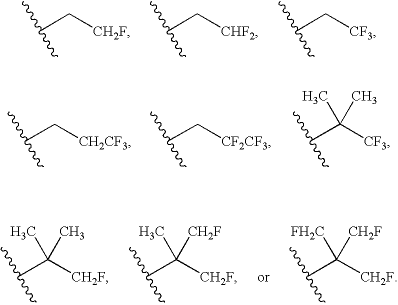

6. The composition according to claim 1, wherein the complex comprises a compound of Formula (I) or a pharmaceutically acceptable salt thereof, wherein:
R¹ is 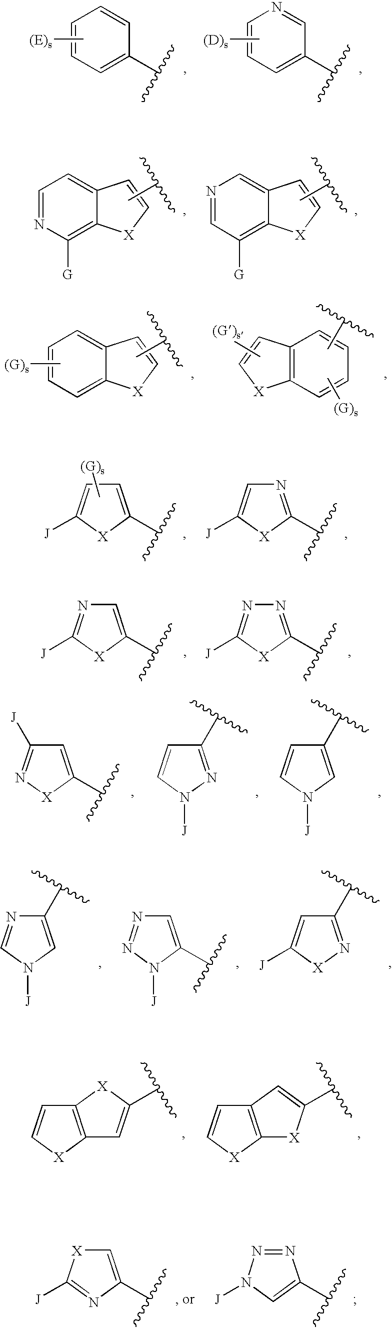
wherein:
each D is independently hydrogen, halogen, cyano, hydroxy, NR^{a}R^{b}, C₁-C₄ alkyl, C₁-C₄ alkoxy, fluorinated C₁-C₄ alkoxy, S-(C₁-C₄ alkyl), phenyl, substituted phenyl, heterocycle, or substituted heterocycle; wherein substituted phenyl is phenyl with one or more subsituents independently selected from halogen, hydroxy, C₁-C₄ alkyl, and C₁-C₄ alkoxy; and wherein substituted heterocycle is heterocycle with one or more substituents independently selected from halogen, hydroxy, C₁-C₄ alkyl, C₁-C₄ alkoxy, fluorinated C₁-C₄ alkoxy, and S-(C₁-C₄ alkyl);
each E is independently hydrogen, halogen, cyano, hydroxy, C₁-C₄ alkyl, C₁-C₄ alkoxy, heterocycle, or substituted heterocycle;
G and G′ are each independently selected from hydrogen, halogen, cyano, hydroxy, C₁-C₄ alkyl, fluorinated C₁-C₄ alkyl, and C₁-C₄ alkoxy;
J is 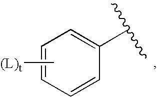
heterocycle, or substituted heterocycle;
each L is independently hydrogen, halogen, cyano, hydroxy, C₁-C₄ alkyl, fluorinated C₁-C₄ alkyl, or C₁-C₄ alkoxy;
X is O or S;
heterocycle in each of D, E and J is independently 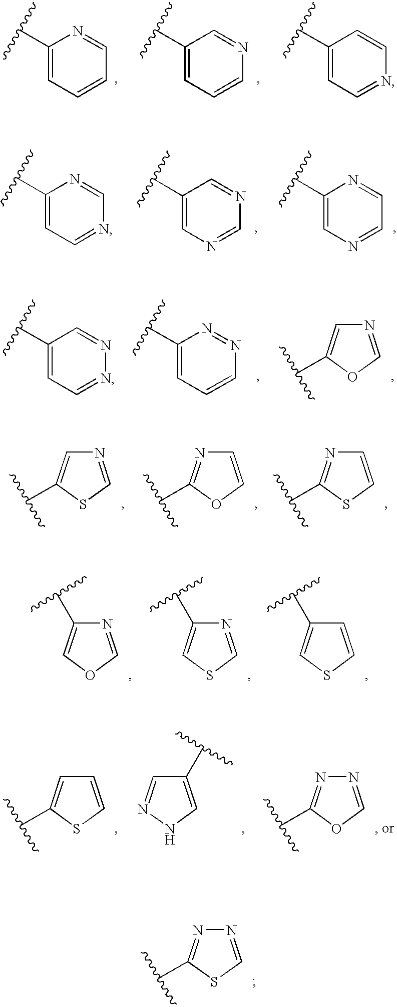
substituted heterocycle in each of E and J is independently heterocycle as defined above with one or more substituents independently selected from halogen, hydroxy, cyano, C₁-C₄ alkyl, fluorinated C₁-C₄ alkyl, C₁-C₄ alkoxy, fluorinated C₁-C₄ alkoxy, S-(C₁-C₄ alkyl), NR^{a}R^{b}, thiazolyl, oxazolyl, imidazolyl, pyrazolyl, triazolyl, pyrrolyl, isoxazolyl, and isothiazolyl;
s, s′, and t are each independently integers from 0 to 2;
R⁴ is 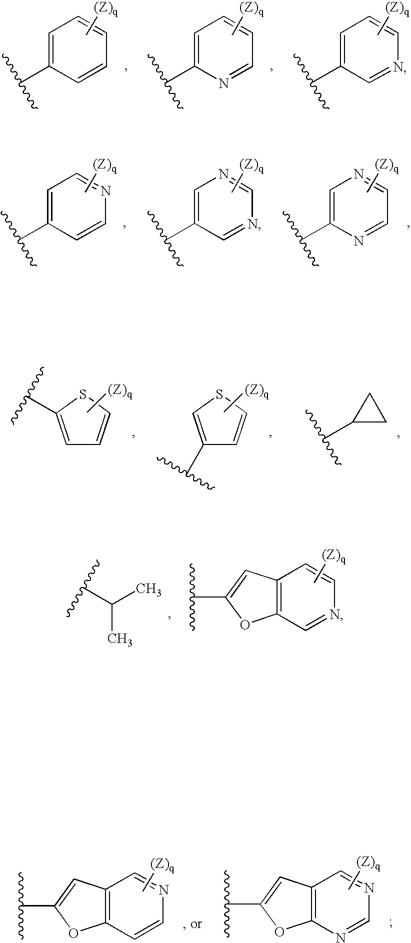
wherein:
each Z is independently hydrogen, halogen, cyano, C₁-C₆ alkyl, or C₁-C₆ alkoxy;
q is an integer from 0 to 2;
R⁵ is 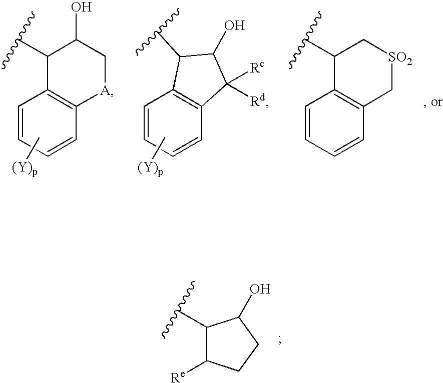
wherein:
A is CR^{c}R^{d}, O, or S;
each Y is independently hydrogen, halogen, C₁-C₆ alkyl, fluorinated C₁-C₆ alkyl, or C₁-C₆ alkoxy;
R^{c} and R^{d} are each independently hydrogen or C₁-C₄ alkyl, or R^{c} and R^{d} together with the carbon to which they are attached form C₃-C₆ cycloalkyl;
R^{e} is hydrogen, C₁-C₄ alkyl, fluorinated C₁-C₄ alkyl, or phenyl; and
p is an integer from 0 to 2; and
R⁶ is

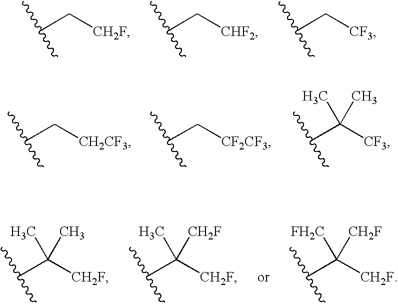

7. The composition according to claim 1, wherein the complex comprises a compound of Formula (I) or a pharmaceutically acceptable salt thereof, wherein:
R¹ is 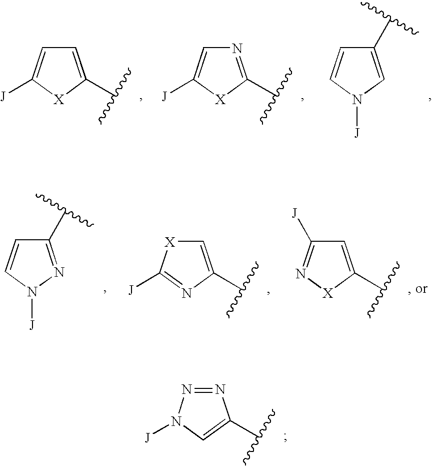
wherein
J is 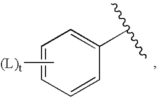
heterocycle, or substituted heterocycle;
each L is independently hydrogen, halogen, cyano, hydroxy, C₁-C₄ alkyl, fluorinated C₁-C₄ alkyl, or C₁-C₄ alkoxy;
t is an integer equal to 0, 1 or 2;
heterocycle is 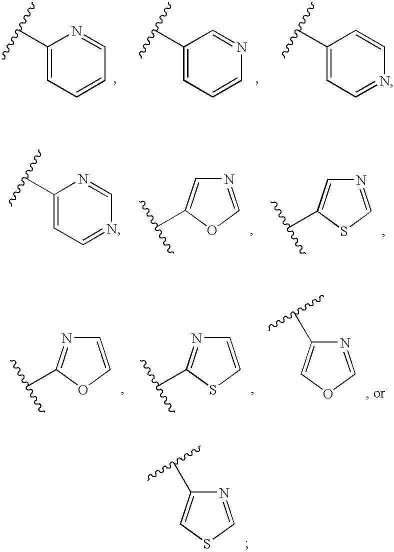
substituted heterocycle is heterocycle as defined above having one or more substituents independently selected from halogen, C₁-C₄ alkoxy, C₁-C₄ alkyl, fluorinated C₁-C₄ alkoxy, fluorinated C₁-C₄ alkyl, —S—CH₃, —N(CH₃)₂, thiazolyl, and oxazolyl; and
X is O or S;
R⁴ is 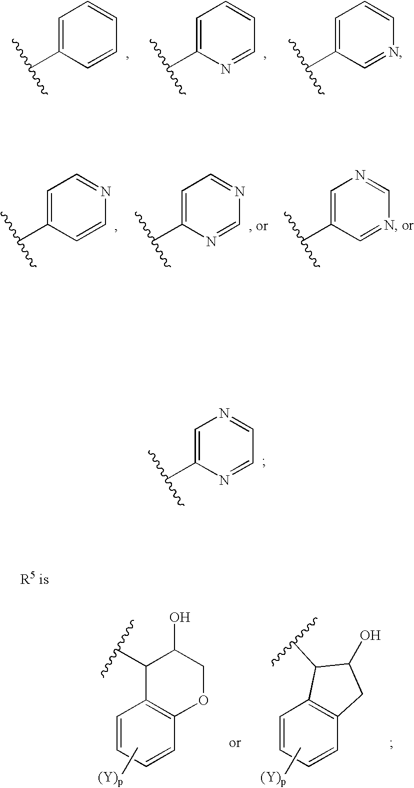
wherein:
each Y is independently hydrogen, halogen, C₁-C₆ alkyl, fluorinated C₁-C₆ alkyl, or C₁-C₄ alkoxy; and
p is an integer from 0 to 2; and
R⁶ is

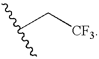

8. The composition according to claim 7, wherein the complex comprises a compound of Formula (I) or a pharmaceutically acceptable salt thereof, wherein:
R¹ is 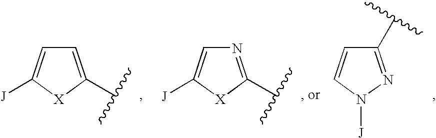
wherein: 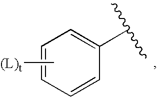
J is
heterocycle, or substituted heterocycle;
heterocycle is 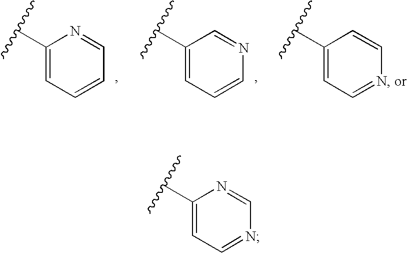
substituted heterocycle is heterocycle as defined above having one or more substituents independently selected from halogen, C₁-C₄ alkoxy, C₁-C₄ alkyl, fluorinated C₁-C₄ alkoxy, fluorinated C₁-C₄ alkyl, —S—CH₃, —N(CH₃)₂, thiazolyl, and oxazolyl; and
X is O or S;
R⁴ is 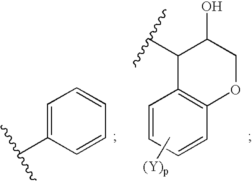
R⁵ is
wherein:
each Y is independently hydrogen, halogen, C₁-C₆ alkyl, fluorinated C₁-C₆ alkyl, or C₁-C₄ alkoxy; and
p is an integer from 0 to 2; and

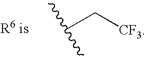

9. The composition according to claim 8, wherein the complex comprises a compound of Formula (I) or a pharmaceutically acceptable salt thereof, wherein:
R² and R³ are each independently hydrogen or methyl;
each L is independently hydrogen, chlorine, or fluorine;
each Y is independently hydrogen, chlorine, or fluorine; and
each of the substituents on substituted heterocycle is independently chlorine, fluorine, methoxy, ethoxy, —OCF₃, —OCHF₂, methyl, ethyl, n-propyl, —S—CH₃, —N(CH₃)₂, and thiazolyl.

10. The composition according to claim 1, wherein the complex comprises a compound selected from the group consisting of:
(αR,γS,2S)-N-((3S,4S)-3,4-dihydro-3-hydroxy-2H-1-benzopyran-4-yl)-4-(1-furo[3,2-c]pyridin-2-yl-1-methylethyl)-γ-hydroxy-α-(phenylmethyl)-2-[[(2,2,2-trifluoroethyl)amino]carbonyl]-1-piperazinepentanamide;
(αR,γS ,2S)-N-((3S,4S)-3,4-dihydro-3-hydroxy-2H-1-benzopyran-4-yl)-γ-hydroxy-4-[(5-phenyl-2-furanyl)methyl]-α-(4-pyridinylmethyl)-2-[[(2,2,2-trifluoroethyl)amino]-carbonyl]-1-piperazinepentanamide;
(αR,γS,2S)-N-((3S,4S)-3,4-dihydro-3-hydroxy-2H-1-benzopyran-4-yl)-γ-hydroxy-4-[1-methyl-1-(1-phenyl-1H-pyrazol-3-yl)ethyl]-α-(3-pyridinylmethyl)-2-[[(2,2,2-trifluoroethyl)amino]carbonyl]-1-piperazinepentanamide;
(αR,γS,2S)-N-((3S,4S)-3,4-dihydro-3-hydroxy-2H-1-benzopyran-4-yl)-γ-hydroxy-α-(phenylmethyl)-4-[[5-(2-pyridinyl)-2-furanyl]methyl]-2-[[(2,2,2-trifluoroethyl)-amino]carbonyl]-1-piperazinepentanamide;
(αR,γS,2S)-4-[[5-(5-chloro-2-pyridinyl)-2-furanyl]methyl]-N-((3S,4S)-3,4-dihydro-3-hydroxy-2H-1-benzopyran-4-yl)-γ-hydroxy-α-(phenylmethyl)-2-[[(2,2,2-trifluoroethyl)amino]carbonyl]-1-piperazinepentanamide;
(αR,γS,2S)-N-(3S,4S)-3,4-dihydro-3-hydroxy-2H-1-benzopyran-4-yl]-γ-hydroxy-4-[1-methyl-1-[5-(3-pyridinyl)-2-oxazolyl]ethyl]-α-(phenylmethyl)-2-[[(2,2,2-trifluoroethyl)amino]carbonyl]-1-piperazinepentanamide;
(αR,γS,2S)-N-(3S,4S)-3,4-dihydro-3-hydroxy-2H-1-benzopyran-4-yl]-γ-hydroxy-4-[1-[5-(5-methoxy-3-pyridinyl)-2-oxazolyl]-1-methylethyl]-α-(phenylmethyl)-2-[[(2,2,2-trifluoroethyl)amino]carbonyl]-1-piperazinepentanamide;
(αR,γS,2S)-N-[(3S,4S)-3,4-dihydro-3-hydroxy-2H-1-benzopyran-4-yl]-γ-hydroxy-4-[1-[5-(5-fluoro-3-pyridinyl)-2-oxazolyl]-1-methylethyl]-α-(phenylmethyl)-2-[[(2,2,2-trifluoroethyl)amino]carbonyl]-1-piperazinepentanamide;
(αR,γS,2S)-N-[(3S,4S)-3,4-dihydro-3-hydroxy-2H-1-benzopyran-4-yl]-γ-hydroxy-4-[1-[1-(5-fluoro-3-pyridinyl)-1H-pyrazol-3-yl]-1-methylethyl]-α-(phenylmethyl)-2-[[(2,2,2-trifluoroethyl)amino]carbonyl]-1-piperazinepentanamide;
(αS,γS,2S)-4-[1-[5-(4-chlorophenyl)-2-oxazolyl]-1-methylethyl]-N-[(3S,4S)-3,4-dihydro-3-hydroxy-2H-1-benzopyran-4-yl]-α-(furo[2,3-c]pyridin-2-ylmethyl)-γ-hydroxy-2-[[(2,2,2-trifluoroethyl)amino]carbonyl]-1-piperazinepentanamide;
(αS,γS,2S)-4-[1-[5-(4-fluorophenyl)-2-oxazolyl]-1-methylethyl]-N-[(3S,4S)-3,4-dihydro-3-hydroxy-2H-1-benzopyran-4-yl]-α-(furo[2,3-c]pyridin-2-ylmethyl)-γ-hydroxy-2-[[(2,2,2-trifluoroethyl)amino]carbonyl]-1-piperazinepentanamide;
(αS,γS,2S)-4-[1-[5-(4-chlorophenyl)-2-oxazolyl]-1-methylethyl]-N-[(3S,4S)-3,4-dihydro-3-hydroxy-2H-1-benzopyran-4-yl]-α-(furo[2,3-c]pyridin-3-ylmethyl)-γ-hydroxy-2-[[(2,2,2-trifluoroethyl)amino]carbonyl]-1-piperazinepentanamide;
(αS,γS,2S)-4-[1-[5-(4-fluorophenyl)-2-oxazolyl]-1-methylethyl]-N-[(3S,4S)-3,4-dihydro-3-hydroxy-2H-1-benzopyran-4-yl]-α-(furo[2,3-c]pyridin-3-ylmethyl)-γ-hydroxy-2-[[(2,2,2-trifluoroethyl)amino]carbonyl]-1-piperazinepentanamide;
(αS,γS,2S)-N-[(3S,4S)-3,4-dihydro-3-hydroxy-2H-1-benzopyran-4-yl]-4-[1-[5-(4-fluorophenyl)-2-oxazolyl]-1-methylethyl]-α-(furo[2,3-d]pyrimidin-6-ylmethyl)-γ-hydroxy-2-[[(2,2,2-trifluoroethyl)amino]carbonyl]-1-piperazinepentanamide;
and pharmaceutically acceptable salts thereof.

11. The composition according to claim 1, wherein the complex comprises Compound A or a pharmaceutically acceptable salt thereof, wherein Compound A is (αR,γS,2S)-4-[[5-(5-chloro-2-pyridinyl)-2-furanyl]methyl]-N-((3S,4S)-3,4-dihydro-3-hydroxy-2H-1-benzopyran-4-yl)-γ-hydroxy-α-(phenylmethyl)-2-[[(2,2,2-trifluoroethyl)amino]carbonyl]-1-piperazinepentanamide.

12. The composition according to claim 1, wherein the cation exchange resin is an acidic sulfonic acid resin or an acidic carboxylic acid resin.

13. The composition according to claim 1, wherein the cation exchange resin is a strongly acidic resin.

14. The composition according to claim 1, wherein the cation exchange resin is an acidic sulfonic acid resin.

15. The composition according to claim 14, wherein the cation exchange resin is Amberlite IRP-69.

16. The composition according to claim 15, wherein the Amberlite IRP-69 is complexed with Compound A or a pharmaceutically acceptable salt thereof, wherein Compound A is (αR,γS,2S)-4-[[5-(5-chloro-2-pyridinyl)-2-furanyl]methyl]-N-((3S,4S)-3,4-dihydro-3-hydroxy-2H-1-benzopyran-4-yl)-γ-hydroxy-α-(phenylmethyl)-2-[[(2,2,2-trifluoroethyl)amino]carbonyl]-1-piperazinepentanamide.

17. The composition according to claim 1, which further comprises a capsule containing the complex.

18. A method of inhibiting HIV protease in a subject in need thereof which comprises administering to the subject a therapeutically effective amount of the composition according to claim 1.

19. A method of preventing or treating infection by HIV in a subject in need thereof which comprises administering to the subject a therapeutically effective amount of the composition according to claim 1.

20. A method of treating or delaying the onset of AIDS in a subject in need thereof which comprises administering to the subject a therapeutically effective amount of the composition according to claim 1.

21. A process for preparing the composition according to claim 1 which comprises:
(A) contacting a cation exchange resin with a compound of Formula (I) in an aqueous or polar organic medium under conditions and for a time sufficient for the compound to form a complex with the resin; and
(B) recovering the complex.

22. The process according to claim 21, wherein the cation exchange resin is an acidic sulfonic acid resin.

23. The composition prepared by the process of claim 22.

24. The complex formed by contacting a cation exchange resin with a compound of Formula (I) as defined in claim 1.
